(19) United States
(12) Patent Application Publication
(10) Pub. No.: US 2012/0202258 A1
(43) Pub. Date: Aug. 9, 2012

(54) LIGE-TYPE TRANSFORMANTS FOR BIOCONVERSION OF LIGNIN-DERIVED COMPOUNDS

(76) Inventors: Ranjini Chatterjee, Belmont, CA (US); Kenneth Zahn, Visalia, CA (US); Kenneth Mitchell, San Jose, CA (US); Yaoquan Liu, Castro Valley, CA (US)

(21) Appl. No.: 13/220,646

(22) Filed: Aug. 29, 2011

Related U.S. Application Data
(60) Provisional application No. 61/403,440, filed on Sep. 16, 2010.
(60) Provisional application No. 61/455,709, filed on Oct. 25, 2010.

(51) Int. Cl. C12P 13/00; C12N 15/63; C12P 7/42; C12P 7/22; C12P 7/24; C12N 15/54; C12N 1/21

(52) U.S. Cl. 435/128; 536/23.2; 435/320.1; 435/252.33; 435/252.3; 435/156; 435/147; 435/146

(57) ABSTRACT

The teachings provided herein are generally directed to a method of converting lignin-derived compounds to valuable aromatic chemicals using an enzymatic, bioconversion process. The teachings provide a selection of (i) host cells that are tolerant to the toxic compounds present in lignin fractions; (ii) polypeptides that can be used as enzymes in the bioconversion of the lignin fractions to the aromatic chemical products; (iii) polynucleotides that can be used to transform the host cells to express the selection of polypeptides as enzymes in the bioconversion of the lignin fractions; and (iv) the transformants that express the enzymes.

### CROSS-REFERENCE TO RELATED APPLICATIONS

This application claims the benefit of U.S. Provisional Application Nos. 61/403,440, filed Sep. 15, 2010; and 61/455,709, filed Oct. 25, 2010; each application of which is hereby incorporated herein by reference in it's entirety,

### SEQUENCE LISTING

The instant application is filed with an ASCII compliant text file of a Sequence Listing. The name of the attached file is ALIGPOO4US01_SEQLIST_AS-FILED.txt, and the file was created Aug. 29, 2011, is 813 KB in size, and is hereby incorporated herein by reference in its entirety. Because the ASCII compliant text file serves as both the paper copy required by §1.821(c) and the CRF required by §1.821(e), the statement indicating that the paper copy and CRF copy of the sequence listing are identical is no longer necessary under 37 C.F.R. §1.821(f), as per Federal Register/Vol. 74, No. 206/Tuesday, Oct. 27, 2009, Section I.

### BACKGROUND OF THE INVENTION

1. Field of the Invention

The teachings provided herein are generally directed to a method of converting lignin-derived compounds to valuable aromatic chemicals using an enzymatic, bioconversion process.

2. Description of the Related Art

Currently, there is a worldwide, global dependence on petroleum as a deplete-able feedstock for the manufacture of fuels and chemicals. The problems of using petroleum are so well-known and documented that they've become nearly a cliché to the world population. In short, petroleum-based processes are dirty and hazardous. Environmental effects associated with the use of petroleum are known to include, for example, air pollution, global warming, damage from extraction, oil spills, tarballs, and health hazards to humans, domestic animals, and wildlife.

Oil refineries, for example, are petroleum-based processes that primarily produce gasoline. However, they are also used extensively to produce valuable and less well-known chemical products used in the manufacture of pharmaceuticals, agrochemicals, food ingredients, and plastics. A clean, green alternative to this market area would be appreciated worldwide.

Bioprocesses can present a clean, green alternative to the petroleum-based processes, a bioprocess being one that uses organisms, cells, organelles, or enzymes to carry out a commercial process. Biorefineries, for example, can produce, for example, chemicals, heat and power, as well as food, feed, fuel and industrial chemical products. Examples of biorefineries can include wet and dry corn mills, pulp and paper mills, and the biofuels industry. In leather tanning, hides are softened and hair is removed using proteases. In brewing, amylases are used in germinating barley. In cheese-making, rennin is used to coagulated the proteins in mil. The biofuels industry, for example, has been a point of focus recently, naturally focusing on fuel products to replace petroleum-based fuels and, as a result, has not developed other valuable chemical products that also rely on petroleum-based processes.

As such, biorefineries use enzymes to convert natural products to useful chemicals. A natural product, such as the wood that is used in a pulp and paper mill, contains cellulose, hemicelluloses, and lignin. A typical range of compositions for a hardwood may be about 40-44% cellulose, about 15-35% hemicelluloses, and about 18-25% lignin. Likewise, a typical range of compositions for a softwood may be about 40-44% cellulose, about 20-32% hemicelluloses, and about 25-35% lignin. Since all biofuels come from cellulosic biorefineries, where the key raw material is glucose, derived from cellulose, lignin remains underutilized. Lignin is the single most abundant source of aromatic compounds in nature, and the use of lignin is currently limited to low value applications, such as combustion to generate process heat and energy for the biorefinery facilities. In the alternative, lignin is sold as a natural component of animal feeds or fertilizers. Interestingly, however, lignin is the only plant biomass component based on aromatic core structures, and such core structures are valuable in the production of industrial chemicals. One of skill will appreciate that, unfortunately, a major problem to such a use of lignin remains: the aromatic compounds present in the lignin fraction of a biorefinery include toxic compounds that inhibit the growth and survival of industrial microbes. For at least these reasons, processes for converting lignin fractions to industrial products using industrial microbes have not been successful.

In view of the above, one of skill will appreciate (i) a clean, green replacement for petroleum-based processes in the production of valuable chemical products that include major markets such as, for example, pharmaceuticals, agrochemicals, food ingredients, and plastics; (ii) a profitable use of the abundant and renewable natural resource available in lignin, which is currently an industrial waste stream that is underutilized as an industrial feedstock; (iii) a selection of host cells that are tolerant to the toxic compounds present in lignin fractions in the feedstock; (iv) a selection of polypeptides that can be used as enzymes in the bioconversion of the lignin fractions to the valuable chemical products; (v) a selection of polynucleotides that can be used to transform host cells to express the selection of polypeptides in the bioconversion of the lignin fractions to the valuable chemical products; (vi) systems that include transformants that express the enzymes, where the transformants can be used to (a) express the enzymes while in direct contact with the lignin fractions or (b) express the enzymes for extraction from the cells, after which the extracted enzymes are used directly in contact with the lignin fractions; and (vii) a clean-and-green method of producing valuable chemical products at higher profits than petroleum-based processes.

### SUMMARY

This invention is generally directed to a recombinant method of producing enzymes for use in the bioconversion of lignin-derived compounds to valuable aromatic chemicals. In some embodiments, the teachings are directed to an isolated recombinant polypeptide, comprising an amino acid sequence having at least 95% identity to SEQ ID NO:101. The sequence can conserve residues T19, I20, S21, P22, V24, W25, T27, K28, Y29, A30, H33, K34, G35, F36, D39, I40, V41, P42, G43, G44, F45, G47, I48, E50, R51, T52, G53, G54, K100, A101, N104, V111, G112, M115, F116, P166, W107, Y184, Y187, R188, G191, G192, and F195.

In some embodiments, the teachings are directed to an isolated recombinant polypeptide, comprising SEQ ID NO:101; or conservative substitutions thereof outside of the conserved residues. The conserved residues can include T19, I20, S21, P22, V24, W25, T27, K28, Y29, A30, H33, K34, G35, F36, D39, I40, V41, P42, G43, G44, F45, G47, I48, E50, R51, T52, G53, G54; K100, A101, N104, V111, G112, M115, F116, P166, W107, Y184, Y187, R188, G191, G192, and F195.

In some embodiments, the teachings are directed to an isolated recombinant glutathione S-transferase enzyme, comprising an amino acid sequence having at least 95% identity to SEQ ID NO:101. The amino acid sequence can conserve residues T19, I20, S21, P22, V24, W25, T27, K28, Y29, A30, H33, K34, G35, F36, D39, I40, V41, P42, G43, G44, F45, G47, I48, E50, R51, T52, G53, G54; K100, A101, N104, V111, G112, M115, F116, P166, W107, Y184, Y187, R188, G191, G192, and F195; wherein, the amino acid sequence functions to cleave a beta-aryl ether.

In some embodiments, the teachings are directed to an isolated recombinant glutathione S-transferase enzyme, comprising an amino acid sequence having at least 95% identity to SEQ ID NO:101; wherein, the amino acid sequence functions to cleave a beta-aryl ether.

In some embodiments, the teachings are directed to an isolated recombinant polypeptide, comprising (i) a length ranging from about 279 to about 281 amino acids; (ii) a first amino acid region consisting of residues 19-54 from SEQ ID NO:101, or conservative substitutions thereof outside of conserved residues T19, I20, S21, P22, V24, W25, T27, K28, Y29, A30, H33, K34, G35, F36, D39, I40, V41, P42, G43, G44, F45, G47, I48, E50, R51, T52, G53, and G54; wherein, the first amino acid region can be located in the recombinant polypeptide from about residue 14 to about residue 59; and, (iii) a second amino acid region consisting of residues 98-221 from SEQ ID NO:101, or conservative substitutions thereof outside of conserved residues K100, A101, N104, V111, G112, M115, F116, P166, W107, Y184, Y187, R188, G191, G192, and F195; wherein, the second amino acid region is located in the recombinant polypeptide from about residue 93 to about residue 226.

In some embodiments, the teachings are directed to an isolated recombinant glutathione S-transferase enzyme, comprising (i) a length ranging from about 279 to about 281 amino acids; (ii) a first amino acid region having at least 95% identity to residues 19-54 from SEQ ID NO:101 while conserving residues T19, I20, S21, P22, V24, W25, T27, K28, Y29, A30, H33, K34, G35, F36, D39, I40, V41, P42, G43, G44, F45, G47, I48, E50, R51, T52, G53, and G54; wherein, the first amino acid region is located in the recombinant polypeptide from about residue 14 to about residue 59; and, (iii) a second amino acid region having at least 95% identity to residues 98-221 from SEQ ID NO:101 while conserving residues K100, A101, N104, V111, G112, M115, F116, P166, W107, Y184, Y187, R188, G191, G192, and F195; wherein, the second amino acid region can be located in the recombinant polypeptide from about residue 93 to about residue 226; and, the recombinant glutathione S-transferase enzyme can function to cleave a beta-aryl ether.

In some embodiments, the teachings are directed to an isolated recombinant glutathione S-transferase enzyme, comprising an amino acid sequence having at least 95% identity to SEQ ID NO:541; wherein, the amino acid sequence functions to cleave a beta-aryl ether.

In some embodiments, the teachings are directed to an isolated recombinant polypeptide, comprising (i) a length ranging from about 256 to about 260 amino acids; (ii) a first amino acid region consisting of residues 47-57 from SEQ ID NO:541, or conservative substitutions thereof outside of conserved residues A47, I48, N49, P50, G52, V54, P55, V56, L57; wherein, the first amino acid region is located in the recombinant polypeptide from about residue 45 to about residue 57; (iii) a second amino acid region consisting of 63-76 from SEQ ID NO:541; and, (iv) a third amino acid region consisting of residues 99-230 from SEQ ID NO:541, or conservative substitutions thereof outside of conserved residues R100, Y101, K104, D107, M111, N112, S115, M116, K176, L194, I197, N198, S201, H202, and M206; wherein, the second amino acid region is located in the recombinant polypeptide from about residue 94 to about residue 235.

In some embodiments, the teachings are directed to an isolated recombinant glutathione S-transferase enzyme, comprising (i) a length ranging from about 279 to about 281 amino acids; (ii) a first amino acid region having at least 95% identity to 47-57 from SEQ ID NO:541, or conservative substitutions thereof outside of conserved residues A47, I48, N49, P50, G52, V54, P55, V56, L57; wherein, the first amino acid region can be located in the recombinant polypeptide from about residue 45 to about residue 57; (iii) a second amino acid region consisting of 63-76 from SEQ ID NO:541; and, (iv) a third amino acid region having at least 95% identity to residues 99-230 from SEQ ID NO:541, or conservative substitutions thereof outside of conserved residues R100, Y101, K104, D107, M111, N112, S115, M116, K176, L194, I197, N198, S201, H202, and M206; wherein, the second amino acid region can be located in the recombinant polypeptide from about residue 94 to about residue 235; wherein, the recombinant glutathione S-transferase enzyme functions to cleave a beta-aryl ether.

In some embodiments, an amino acid substitution outside of the conserved residues can be a conservative substitution. And, in many embodiments, the amino acid sequence can function to cleave a beta-aryl ether.

The teachings are also directed to a method of cleaving a beta-aryl ether bond, the comprising contacting a polypeptide taught herein with a lignin-derived compound having (i) a beta-aryl ether bond and (ii) a molecular weight ranging from about 180 Daltons to about 3000 Daltons; wherein, the contacting occurs in a solvent environment in which the lignin-derived compound is soluble.

In some embodiments, the lignin-derived compound has a molecular weight of about 180 Daltons to about 1000 Daltons. In some embodiments, the solvent environment comprises water. And, in some embodiments, the solvent environment comprises a polar organic solvent.

The teachings are also directed to a system for bioprocessing lignin-derived compounds, the system comprising a polypeptide taught herein, a lignin-derived compound having a beta-aryl ether bond and a molecular weight ranging from about 180 Daltons to about 3000 Daltons; and, a solvent in which the lignin-derived compound is soluble; wherein, the system functions to cleave the beta-aryl ether bond by contacting the polypeptide with the lignin-derived compound in the solvent.

The teachings are also directed to a recombinant polynucleotide comprising a nucleotide sequence that encodes a polypeptide taught herein. Likewise, the teachings are also directed to a vector or plasmid comprising the polynucleotide, as well as a host cell transformed by the vector or plasmid to express the polypeptide.

The teachings are also directed to a method of cleaving a beta-aryl ether bond, the method comprising (i) culturing a host cell taught herein under conditions suitable to produce a polypeptide taught herein; (ii) recovering the polypeptide from the host cell culture; and, (iii) contacting the polypeptide of claim 1 with a lignin-derived compound having a beta-aryl ether bond and a molecular weight ranging from about 180 Daltons to about 3000 Daltons; wherein, the contacting occurs in a solvent environment in which the lignin-derived compound is soluble.

In some embodiments, the host cell can be E. Coli or an Azotobacter strain, such as Azotobacter vinelandii. And, in some embodiments, the lignin-derived compound can have a molecular weight of about 180 Daltons to about 1000 Daltons.

The teachings are also directed to a system for bioprocessing lignin-derived compounds, the system comprising (i) a transformed host cell taught herein; (ii) a lignin-derived compound having a beta-aryl ether bond and a molecular weight ranging from about 180 Daltons to about 3000 Daltons; and, (iii) a solvent in which the lignin-derived compound is soluble; wherein, the system functions to cleave the beta-aryl ether bond by contacting a polypeptide taught herein with the lignin-derived compound in the solvent.

### BRIEF DESCRIPTION OF THE DRAWINGS

FIGS. 1A and 1B illustrate general concepts of the biorefinery and discovery processes discussed herein, according to some embodiments.

FIG. 2 illustrates the structures of some building block chemicals that can be produced using bioconversions, according to some embodiments.

FIG. 3 is an example of a beta-etherase catalyzed hydrolysis of a model lignin dimer, α-O-(β-methylumbelliferyl) acetovanillone (MUAV), according to some embodiments.

FIG. 4 illustrates unexpected results from biochemical activity assays for beta-etherase function for the S. paucimobilis positive control polypeptides, and the N. aromaticivorans putative beta-etherase polypeptide, according to some embodiments.

FIG. 5 illustrates beta-aryl-ether compounds to be tested as substrates representing native lignin structures, according to some embodiments.

FIG. 6 illustrates pathways of guaiacylglycerol-β-guaiacyl ether (GGE) metabolism by S. paucimobilis, according to some embodiments.

FIG. 7 illustrates an example of a biochemical process for the production of catechol from lignin oligomers, according to some embodiments.

FIG. 8 illustrates an example of a biochemical process for the production of vanillin from lignin oligomers, according to some embodiments.

FIG. 9 illustrates an example of a biochemical process for the production of 2,4-diaminotoluene from lignin oligomers, according to some embodiments.

FIG. 10 illustrates process schemes for additional product targets that include ortho-cresol, salicylic acid, and aminosalicylic acid, for the production of valuable chemicals from lignin oligomers, according to some embodiments.

### DETAILED DESCRIPTION OF THE INVENTION

This invention is generally directed to a recombinant method of producing enzymes for use in the bioconversion of lignin-derived compounds to valuable aromatic chemicals. Currently, the art is limited in it's ability to control the degradation of lignin to produce useful products, as it's limited in it's knowledge of enzymes that are capable of selectively converting lignin into desired aromatic compounds. Generally, the art knows two basic things: (1) lignin is complex; and (2) bacterial lignin degradation systems are therefore at least as complex as lignin itself. Accordingly, and for at least these reasons, the teachings provided herein offer a valuable, unexpected, and surprising set of systems, methods, and compositions of matter that will be useful in the production of industrially useful aromatic chemicals.

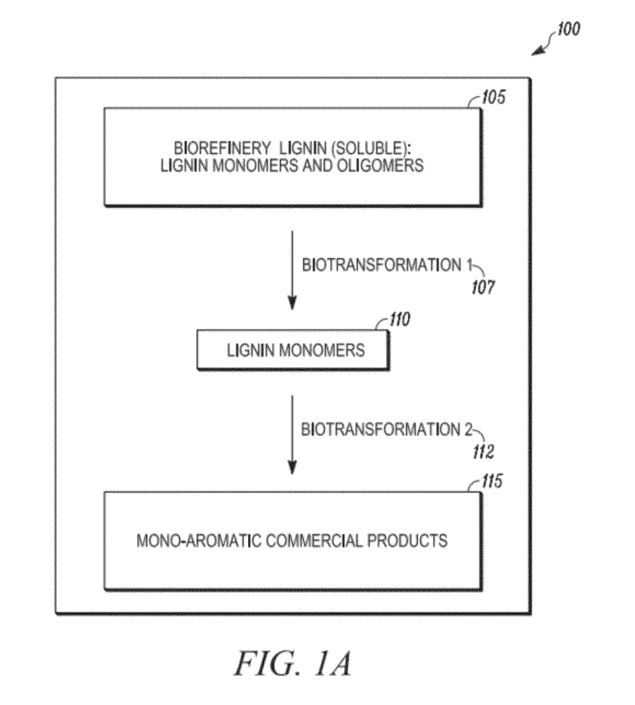

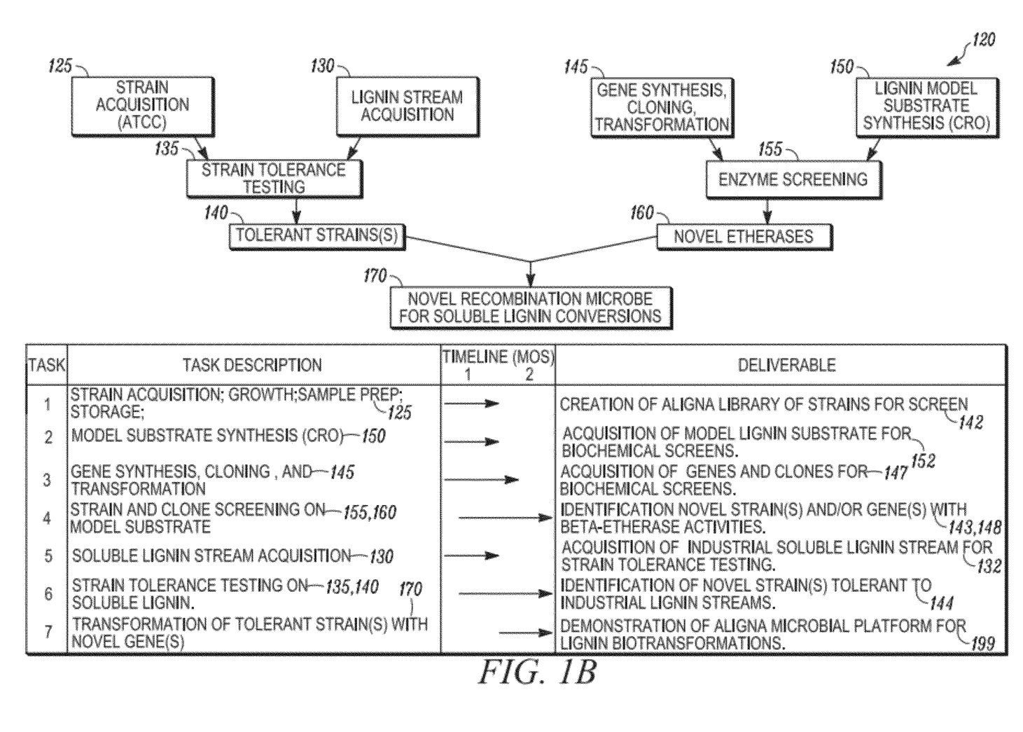

FIGS. 1A and 1B illustrate general concepts of the biorefinery and discovery processes discussed herein, according to some embodiments. FIG. 1A shows a generalized example of a use of recombinant microbial strains in biotransformations for the production of aromatic chemicals from lignin-derived compounds. Biorefinery process 100 converts a soluble biorefinery lignin 105 through a series of biotransformations using a transformed host cell. The biorefinery lignin 105 is a feedstock comprising a lignin-derived compound which can be, for example, a combination of lignin-derived monomers and oligomers.

(ATCC 31,537); E. coli stran be used to selectively cleave a bond on or between monomers to create additional lignin monomers 110. “Biotransformation 2” 112 can be used to selectively cleave an additional bond on or between monomers to create mono-aromatic commercial products 115. FIG. 1B shows a discovery process 120, which includes selecting a host cell strain that is tolerant to toxic lignin-derived compounds. The strain acquisition 125 includes growth of the strain, sample preparation, and storage. A set of bacterial strains are obtained for testing strain tolerance to soluble biorefinery lignin samples.

In some embodiments, the strains can be selected for (i) having well-characterized aromatic and xenobiotic metabolisms; (ii) annotated genome sequences; and (iii) prior use in fermentation processes at pilot or larger scales. Examples of strains can include, but are not limited to, Azotobacter vinelandii (ATCC BAA-1303 DJ), Azotobacter chroococcum (ATCC 4412 (EB Fred) X-50), Pseudomonas putida (ATCC BAA-477 Pf-5), Pseudomonas fluorescens (ATCC 29837 NCTC 1100). Stains can be streaked on relevant rich media plates as described by the accompanying ATCC literature for revival. Individual colonies (5 each) can be picked and cultured on relevant liquid media to saturation. Culture samples prepared in a final glycerol concentration of 12.5% can be flash-frozen and stored at −80° C.

The model substrate synthesis 150 for use in the biochemical screening for selective activity can be outsourced through a contract research organization (CRO). The enzyme discovery effort can initially be focused on identifying potential beta-etherase candidate genes identified through bioinformatic methods. The identification of candidates having beta-etherase activity is the 1st step towards generating lignin monomers from lignin oligomers present in soluble lignin streams. The fluorescent substrate α-O-(β-methylumbelliferyl) acetovanillone (MUAV), for example, can be used in in vitro assays to identify beta-etherase function (Acme Biosciences, Mt. View, Calif.). The formation of 4 methylumbelliferone (4MU) upon hydrolysis of the aryl ether bond can be monitored by fluorescence, for example, at λex=365 nm and λem=450 nm (or 460 nm).

The gene synthesis, cloning, and transformation step 145 can include combining bioinformatic methods with known information about enzymes showing a desired, selective enzyme activity. For example, bioinformatics can produce a putative beta-etherase sequence that shares a significant homology to the S. paucimobilis ligE and ligF beta-etherase sequences. See Masai, E., et al. Journal of Bacteriology (3):1768-1775 (2003)(“Masai”), which is hereby incorporated herein in it's entirety by reference. The S. paucimobilis sequences can be used as positive controls for biochemical assays to show relative activities in an enzyme discovery strategy.

The gene synthesis, cloning, and transformation step 145 can be performed using any method known to one of skill. For example, all genes can be synthesized directly as open reading frames (ORFs) from oligonucleotides by using standard PCR-based assembly methods, and using the E. coli codon bias. The end sequences can contain adaptors (BamHI and HindIII) for restriction digestion and cloning into the E. coli expression vector pET24a (Novagen). Internal BamHI and HindIII sites can be excluded from the ORF sequences during design of the oligonucleotides. Assembled genes can be cloned into the proprietary cloning vector (pGOV4), transformed into E. coli CH₃ chemically competent cells, and DNA sequences determined (Tocore Inc.) from purified plasmid DNA. After sequence verification, restriction digestion can be used to excise each ORF fragment from the cloning vector, and the sequence can be sub-cloned into pET24a. The entire set of ligE and ligF bearing plasmids can then be transformed into E. coli BL21 (DE3) which can serve as the host strain for beta-etherase expression and biochemical testing.

The enzyme screening 155 is done to identify novel etherases 160. The fluorescent substrate MUAV can be used to screen for and identify beta-etherase activity from the recombinant E. coli clones. Expression of the beta-etherase genes can be done in 5 ml or 25 ml samples of the recombinant E. coli strains in LB medium using induction with IPTG. Following induction, and cell harvest, cell pellets can be be lysed using the BPER (Invitrogen) cell lysis system. Cell extracts can be tested in the in vitro biochemical assay for beta-etherase activity on the fluorescent substrate MUAV. The formation of 4 methylumbelliferone (4MU) upon hydrolysis of the aryl ether bond in MUAV can be monitored by fluorescence at λex=365 nm and λem=460 nm, and can provide quantitative measurement of beta-etherase function. Cell extracts of E. coli transformed with the S. paucimobilis ligE and ligF genes can be the assay positive controls. Test or unknown samples can include, for example, E. coli strains expressing putative beta-etherase genes from N. aromaticovorans.

The lignin stream acquisition 130 includes a waste lignin stream from a biorefinery for testing. A preliminary characterization of one source of such lignin has shown an aromatic monomer concentration of less than 1 g/L and an oligomer concentration of ˜10 g/L. Oligomers appear to be associated with carbohydrates in 10:1 ratio for sugar:phenolics. Some information exists on compounds in the liquid stream, including benzoic acid, vanillin, syringic acid and ferulics, which are routinely quantified in soluble samples. An average molecular weight of ˜280 has been established for the monomers; and the oligomeric components remain to be characterized.

The strain tolerance testing 135 Strain tolerance will be determined by cell growth upon exposure to biorefinery lignin. Tolerance to the phenolic compounds in biorefinery lignin waste stream will be critically important to the bioprocess efficiency and high level production of aromatic chemicals by microbial systems. Cell growth will be quantified as a function of respiration by the reduction of soluble tetrazolium salts. XTT (2,3-Bis(2-methoxy-4-nitro-5-sulfophenyl)-2H-tetrazolium-5-carboxanilide inner salt, Sigma) is reduced to a soluble purple formazan compound by respiring cells. The formazan product will be detected and quantified by absorbance at 450 nm.

Strain tolerance testing 135 on soluble lignin can be done in liquid format in 48 well plates, for example. Each strain can be tested in replicates of 8, for example, and E. coli can be used as a negative control strain. Strains can first be grown in rich medium to saturation, washed, and OD600 nm of the cultures determined. Equal numbers of bacteria can be inoculated into wells of the 48-well growth plate containing minimal medium excluding a carbon source. Increasing concentrations of soluble lignin fractions, in addition to a minus-lignin positive control, can be added to the wells containing each species to a final volume of 0.8 ml. A benzoic acid content analysis of the lignin fractions can be used as an internal indicator of the phenolic content of lignin wastes of different origin. Following incubation for 24-48 hours with shaking at 30° C., the cultures can be tested for growth upon exposure to the lignin fraction using an XTT assay kit. Culture samples can be removed from the 48 well growth plate and diluted appropriately in 96 well assay plates to which the XTT reagent can be added. The soluble formazan produced will be quantified by absorbance at 450 nm. Bacterial strains exhibiting the highest level of growth, and therefore tolerance, can be candidates for further development as host strains for lignin conversions.

The strain demonstrated to have the best tolerance characteristics can be transformed with the beta-etherase gene identified as showing the highest biochemical activity. Restriction digestion can be used to excise the ORF fragment from the cloning vector, and the sequence can be sub-cloned into the shuttle vector pMMB206. Constructs cloned in the shuttle vector can be transformed into Azotobacter or Pseudomonas strains by electroporation, or chemical transformation. The recombinant, lignin tolerant host strain can be re-tested for beta-etherase expression and activity using any methods known to one of skill, such as those described herein, adapted to the particular host strain being used.

Feedstock from Biorefinery Processes

An example of a starting material might be pretreated lignocellulosic biomass. In some embodiments, the lignocellulose biomass material might include grasses, corn stover, rice hull, agricultural residues, softwoods and hardwoods. In some embodiments, the lignin-derived compounds might be derived from hardwood species such as poplar from the Upper Peninsula region of Michigan, or hardwoods such as poplar, lolloby pine, and eucalyptus from Virginia and Georgia areas, or mixed hardwoods including maple and oak species from upstate New York.

In some embodiments, the pretreatment methods might encompass a range of physical, chemical and biological based processes. Examples of pretreatment methods used to generate the feedstock for Aligna processes might include physical pretreatment, solvent fractionation, chemical pretreatment, biological pretreatment, ionic liquids pretreatment, supercritical fluids pretreatment, or a combination thereof, for example, which can be applied in stages.

Physical pretreatment methods used to reduce the lignocellulose biomass particle size reduction might utilize mechanical stress methods of dry, wet vibratory and compression-based ball milling procedures. Solvent fractionation methods include organosolve processes, phosphoric acid fractionation processes, and methods using ionic liquids to pretreat the lignocellulose biomass to differentially solubilize and partition various components of the biomass. In some embodiments, organosolve methods might be performed using alcohol, including ethanol, with an acid catalyst at temperature ranges from about 90 to about 20° C., and from about 155 to about 220° C. with residence time of about 25 minutes to about 100 minutes. Catalyst concentrations can vary from about 0.83% to about 1.67% and alcohol concentrations can vary from about 25% to about 74% (v/v). In some embodiments, phosphoric acid fractionations of lignocellulose biomass might be performed using a series of different extractions using phosphoric acid, acetone, and water at temperature of around 50° C. In some embodiments, ionic liquid pretreatment of lignocellulose biomass might include use of ionic liquids containing anions like chloride, formate, acetate, or alkylphosphonate, with biomass:ionic liquids ratios of approximately 1:10 (w/w). The pretreatment might be performed at temperatures ranging from about 100° C. to about 150° C. Other ionic liquid compounds that might be used include 1-butyl-3-methyl-imidazolium chloride and 1-ethyl-3-methylimidazolium chloride.

Chemical pretreatments of lignocellulose biomass material might be performed using technologies that include acidic, alkaline and oxidative treatments. In some embodiments, acidic pretreatment methods of lignocellulose biomass such as those described below might be applied. Dilute acid pretreatments using sulfuric acid at concentrations in the approximate range of about 0.05% to about 5%, and temperatures in the range of about 160° C. to about 220° C. Steam explosion, with or without the use of catalysts such as sulfuric acid, nitric acid, carbonic acid, succinic acid, fumaric acid, maleic acid, citric acid, sulfur dioxide, sodium hydroxide, ammonia, before steam explosion, at temperatures between about 160° C. to about 290° C. Liquid hot water treatment at pressure >5 MPa at temperatures ranging from about 160° C. to about 230° C., and pH range between about 4 and about 7. And, in some embodiments, alkaline pretreatment methods using catalysts such as calcium oxide, ammonia, and sodium hydroxide might be used. The ammonia fiber expansion (AFEX) method might be applied in which concentrated ammonia at about 0.3 kg to about 2 kg of ammonia per kg of dry weight biomass is used at about 60° C. to about 140° C. in a high pressure reactor, and cooked for 5-45 minutes before rapid pressure release. The ammonia recycle percolation (ARP) method might be used in flow through mode by percolating ammoniacal solutions at 5-15% concentrations at high temperatures and pressures. Oxidative pretreatment methods such as alkaline wet oxidation might be used with sodium carbonate at a temperature ranging from about 170° C. to about 220° C. in a high pressure reactor using pressurized air/oxygen mixtures or hydrogen peroxide as the oxidants.

Biological pretreatment methods using white rot basidomycetes and certain actinomycetes might be applied. One type of product stream from such pretreatment methods might be soluble lignin, and might contain lignin-derived monomers and oligomers in the range of about 1 g/L to about 10 g/L, and xylans. The lignin-derived monomers might include compounds such as gallic acid, hydroxybenzoate, ferulic acid, hydroxymethyl furfural, hydroxymethyl furfural alcohol, vanillin, homovanillin, syringic acid, syringaldehyde, and furfural alcohol.

Supercritical fluid pretreatment methods might be used to process the biomass. Examples of supercritical fluids for use in processing biomass include ethanol, acetone, water, and carbon dioxide at a temperature and pressures above the critical points for ethanol and carbon dioxide but at a temperature and/or pressure below that of the critical point for water.

Combinations of steam pretreatment and biological pretreatment methods might be applied. For example, a biomass steam can be pretreated at 195° C. for 10 min at controlled pH, followed by enzymatic treatment using commercial cellulases and xylanases at dosings of 100 mg protein/g total solid, and with incubation at 50° C. at pH 5.0 with agitation of 500 rpm.

In some embodiments, combinations of hydrothermal, organosolve, and biological pretreatment methods might be used. One example of such a combination is a 3 stage process:

Stage 1. Use heat in an aqueous medium at a predetermined pH, temperature and pressure for the hydrothermal process;
Stage 2. Use at least one organic solvent from those described in 6-6c in water for the organosolve step;
Stage 3. Use yeast, white rot basidomycetes, actinomycetes, and cellulases and xylanases in native or recombinant forms for the biological pretreatment step.

Soluble lignin fractions derived using organosolve methods might produce soluble lignins in the molecular weight range of 188-1000, soluble in various polar solvents. Without intending to be bound by any theory or mechanism of action, organosolve processes are generally believed to maintain the lignin beta-aryl ether linkage.

Lignin streams from steam exploded lignocellulosic biomass might be used. Steam explosion might be performed, for example, using high pressure steam in the range of about 200 psi to about 500 psi, and at temperatures ranging from about 180° C. to about 230° C. for about 1 minute to about 20 minutes in batch or continuous reactors. The lignin might be extracted from the steam-exploded material with alkali washing or extraction using organic solvents. Steam exploded lignins can exhibit properties similar to those described form organosolve lignins, retaining native bond structures and containing about 3 to about 12 aromatic units per oligomer unit.

Supercritical fluid pretreatment can produce soluble lignin fractions that can be used with the teachings provided herein. Such processes typically yield monomers and lignin oligomers having a molecular weight of about <1000 Daltons.

Biological pretreatment can produce soluble lignin fractions that can be used with the teachings provided herein. Such lignin streams might contain lignin monomers and oligomers in the range of about 1 g/L to about 10 g/L and have a molecular weight of about <1000 Daltons, and xylans. The lignin-derived monomers might include compounds such as gallic acid, hydroxybenzoate, ferulic acid, hydroxymethyl furfural, hydroxymethyl furfural alcohol, vanillin, homovanillin, syringic acid, syringaldehyde, and furfural alcohol.

Feedstock from Wood Pulping Processes

Wood pulping processes produce a variety of lignin types, the type of lignin dependent on the type of process used. Chemical pulping processes include, for example, Kraft and sulfite pulping.

In some embodiments, the lignin-derived compound can be derived from a spent pulping liquor or “black liquor” from Kraft pulping processes. Kraft lignin might be derived from batch or continuous processes using, for example, reaction temperatures in the range of about 150° C. to about 200° C. and reaction times of approximately 2 hours. Any range of molecular weights of lignin may be obtained, and the useful fraction may range, in some embodiments, from about 200 Daltons to about 4000 Daltons. A Kraft lignin having a molecular weight ranging from about 1000 Daltons to about 3000 Daltons might be used in a bioconversion.

In some embodiments, lignin from a sulfite pulping process might be used. A sulfite pulping process can include, for example, a chemical sulfonation using aqueous sulfur dioxide, bisulfite and monosulfite at a pH ranging from about 2 to about 12. The sulfonated lignin might be recovered by precipitation with excess lime as lignosulfonates. Alternatively, formaldehyde-based methylation of the lignin aromatics followed by sulfonation might be performed. Any range of molecular weights of lignin may be obtained, and the useful fraction may range, in some embodiments, from about 200 Daltons to about 4000 Daltons. A sulfite lignin having a molecular weight ranging from about 1000 Daltons to about 3000 Daltons might be used in a bioconversion.

### Characterization of Lignin-Derived Compounds for Use in Bioconversion

Optimization of a system for a particular feedstock should include an understanding of the composition of the particular feedstock. For example, one of skill will appreciate that the composition of a native lignin can be significantly different than the composition of the lignin-derived compounds in a given lignin faction that is used for a feedstock. Accordingly, and understanding of the composition of the feedstock will assist in optimizing the conversion of the lignin-derived compounds to the valuable aromatic compounds. Any method known to one of skill can be used to characterize the compositions of the feedstock. For example, one of skill may use wet chemistry techniques, such as thioacidolysis and nitrobenzene oxidation, coupled with gas chromatography, which have been used traditionally, or spectroscopic techniques such as NMR and FTIR. Thioacidolysis, for example, cleaves the β-O-4 linkages in lignin, giving rise to monomers and dimers which are then used to calculate the S and G content. Similar information can be obtained using nitrobenzene oxidation, but the ratios are thought to be less accurate. In some embodiments, the content of S, G, and H, as well as their relative ratios can be used to characterize feedstock compositions for purposes of determining a bioconversion system design.

It is widely accepted that the biosynthesis of lignin stems from the polymerization of three types of phenylpropane units, also referred to as monolignols. These units are coniferyl, sinapyl, and p-coumaryl alcohol. The three structures are as follows:

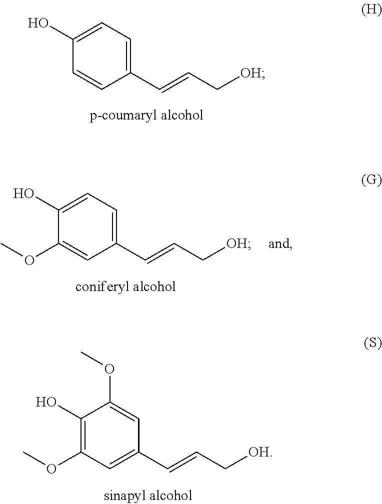

Tables 1A and 1B summarize distributions of p-coumaryl alcohol or p-hydroxyl phenol (H), coniferyl alcohol or guaiacyl (G), and sinapyl alcohol or syringyl (S) lignin in several sources of biomass. Table 1A compares percent lignin in the biomass to the G:S:H.

TABLE 1A

| | % Lignin | G: | S: | H |
|---|---|---|---|---|
| Wheat Straw | 16-21 | 45 | 46 | 9 |
| Rice Straw | 6 | 45 | 40 | 15 |
| Rye Straw | 18 | 43 | 53 | 1 |
| Hemp | 8-13 | 51 | 40 | 9 |
| Tall Fescue: | | | | |
| Stems | 7-10 | 55 | 42 | 3 |
| Internodes | 11 | 48 | 50 | 2 |
| Flax | 21-34 | 67 | 29 | 4 |
| Jute | 15-26 | 36 | 62 | 2 |
| Sisal | 7-14 | 22 | 76 | 2 |
| Curaua Leaf fiber | 7 | 29 | 41 | 30 |
| Banana Plant Leaf | | 43 | 50 | 7 |
| Piassava Fiber | 45 | 40 | 9 | 51 |
| (Plam Tree) | | | | |
| Abaca | 7-9 | 19 | 55 | 26 |
| Loblolly Pine | 29 | 86 | 2 | 12 |
| | 29 | 87 | 0 | 13 |
| Compression | | 60 | | 40 |
| Spruce (Picea Abies) | 28 | 94 | 1 | 5 |
| MWL | | 98 | 2 | 0 |
| Eucalyptus globus | 22 | 14 | 84 | 2 |
| Eucalyptus grandis | 27 | 27 | 69 | 4 |
| Birch pendula | 22 | 29 | 69 | 2 |
| Beech | 26 | 56 | 40 | 4 |
| Acacia | 28 | 48 | 49 | 3 |

TABLE 1B

| White Birch | G: | S |
|---|---|---|
| Fiber, S2 layer | 12 | 88 |
| Vessel, S2 Layer | 88 | 12 |
| Ray parenchyma, S-layer | 49 | 51 |
| Middle lamella (fiber/fiber) | 91 | 9 |
| Middle lamella (fiber/vessel) | 80 | 20 |
| Middle lamella (fiber/ray) | 100 | 0 |
| Middle lamella (ray/ray) | 88 | 12 |

| | G: | S: | H |
|---|---|---|---|
| Lignin Samples [symbol] | | | |
| Carpinus betulus MWL | 19 | 80 | 1 |
| Eucryphia codrifolia MWL | 35 | 59 | 6 |
| Bambusa sp. MWL | 23 | 57 | 20 |
| Fagus sylvatica kraft lignin | 25 | 72 | 3 |
| Eucalyptus globulus kraft lignin | 22 | 73 | 6 |
| Lobolly Pine Juvenile | | | |
| Normal | 95 | | 5 |
| Wind Opposite | 96 | | 4 |
| Wind Compression | 89 | | 11 |
| Bent Opposite | 96 | | 4 |
| Bent Compression | 88 | | 12 |

[symbol]

indicates data missing or illegible when filed

In general, the relative amounts of G, S, and H in lignin can be a good indicator of its overall composition and response to a treatment, such as the bioconversions taught herein. In poplar species, for example, differences can be seen based on the measurement technique as well as species, but in general the S/G ratio ranges from 1.3 to 2.2. This is similar to the hardwood eucalyptus, but higher than herbaceous biomass switchgrass and Miscanthus. This is to be expected given the higher H contents in grass lignin. An optimized nitrobenzene oxidation method has shown S/G ratios of 13 poplar samples from two different sites and obtained values ranging from 1.01 to 1.68. Further, a linear correlation (R²=0.85) has been found in poplar between decreasing lignin content and increasing S/G ratios. The correlation was stronger (R²=0.93) in samples from a single site suggesting a dependency on geographic location.

Higher throughput methods can be used for rapid screening of feedstocks. Examples of such methods can include, but are not limited to, near-infrared (NIR), reflectance spectroscopy, pyrolysis molecular beam mass spectrometry (pyMBMS), Fourier transform infrared spectroscopy, a modified thioacidolysis technique, and whole cell NMR after dissolution in ionic liquids. Information on some structural characteristics of lignin, such as S/G ratios, can be rapidly obtained using these methods. The average S:G:H ratio of 104 poplar lignin samples, for example, was determined using the modified thioacidolysis technique, and was found to be 68:32:0.02. In some embodiments, the S, G, and H components in the ratio can be expressed as mass percent. In some embodiments, the S, G, and H components in the ratio can be expressed as any relative unit, or unitless. Any comparison can be used, if the amount of each component directly correlates with the other respective components in the composition. The ratios can be expressed in relative whole numbers or fractions as S:G:H, or any other order or combination of components, S/G, G/S, and the like. In some embodiments, the S/G ratio is used. In some embodiments, the S/G ratio can range from about 0.20 to about 20.0, from about 0.3 to about 18.0, from about 0.4 to about 15.0, from about 0.5 to about 15.0, from about 0.6 to about 12.0, from about 0.7 to about 10.0, from about 0.8 to about 8.0, from about 0.9 to about 9.0, from about 1.0 to about 7.0, or any range therein. In some embodiments, the S/G ratio can be about 0.2, about 0.4, about 0.6, about 0.8, about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, about 6.2, about 6.4, about 6.6, about 6.8, about 7.0, about 7.2, about 7.4, about 7.6, about 7.8, about 8.0, about 8.2, about 8.4, about 8.6, about 8.8, about 9.0, about 9.2, about 9.4, about 9.6, about 9.8, about 10.0, and any ratio in-between on 0.1 increments, and any range of ratios therein.

### Fractionation of Lignin-Derived Compounds for Use in Bioconversion

Soluble lignin streams derived from biorefinery or Kraft processes might be used directly in microbial conversions without additional purification or, they might be further purified by one or more of the separation or fractionation techniques prior to microbial conversions.

In some embodiments, membrane filtration might be applied to achieve a starting concentration of lignin monomers and oligomers in the 1-60% (w/v) concentration range, and molecular weights ranging from about 180 Daltons to about 2000 Daltons, from about 200 Daltons to about 4000 Daltons, from about 250 Daltons to about 2500 Daltons, from about 180 Daltons to about 3500 Daltons, from about 300 Daltons to about 3000 Daltons, or any range therein.

In some embodiments, soluble lignin streams might be partially purified by chromatography using, for example, HP-20 resin. The lignin monomers and oligomers can bind to the resin while highly polar impurities or inorganics that might be toxic to microorganisms can remain un-bound. Subsequent elution, for example, with a methanol-water solvent system, can provide fractions of higher purity that are enriched in lignin monomers and oligomers.

### Chemical Products

A purpose of the present teaching includes the discovery of novel biochemical conversions that create valuable commercial products from various lignin core structures. Such commercial products include monomeric aromatic chemicals that can serve as building block chemicals. One of skill will appreciate that a vast number of aromatic chemicals can be produced using the principles provided by the teachings set-forth herein, and that a comprehensive teaching of every possible chemical that can be produced would be beyond the scope and purpose of this teaching.

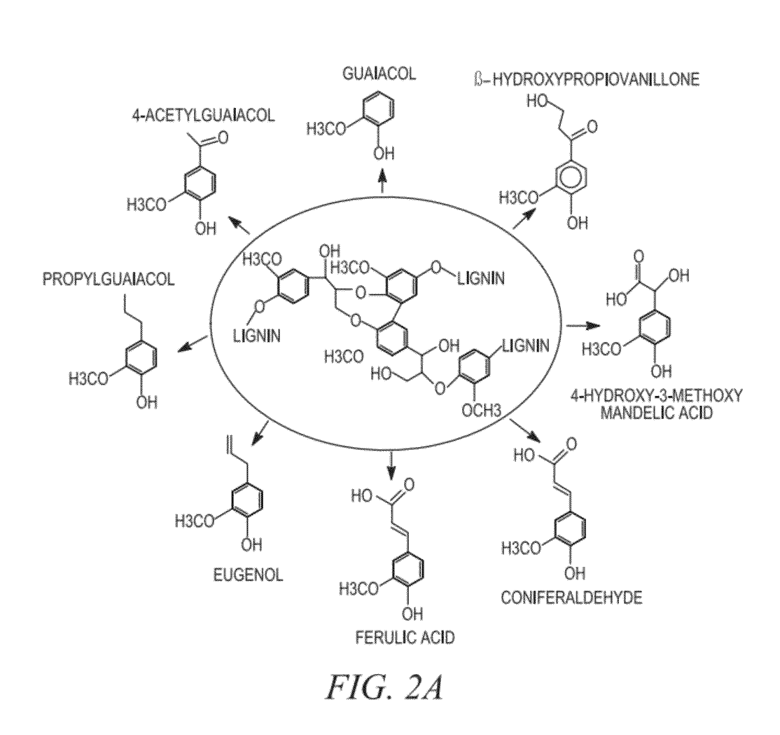

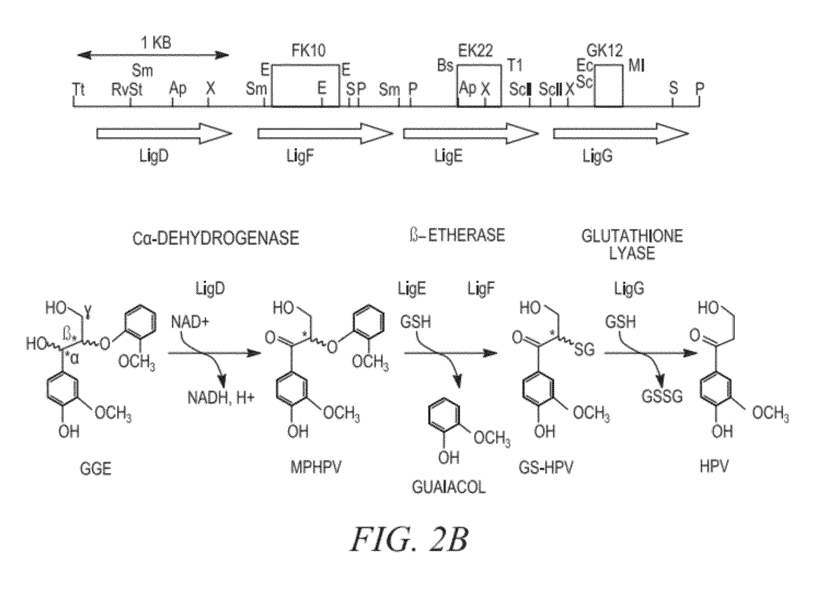

FIGS. 2A and 2B illustrate (i) the structures of some building block chemicals that can be produced using bioconversions, and (ii) an example enzyme system from a Sphingomonaas paucimobilis gene cluster, according to some embodiments. FIG. 2A shows that examples of some monomeric aromatic structures that can serve as building block chemicals derived from lignin include, but are not limited to, guaiacol, β-hydroxypropiovanillone, 4-hydroxy-3methoxy mandelic acid, coniferaldehyde, ferulic acid, eugenol, propylguaicol, and 4-acetylguaiacol. It should be appreciated that each of these structures can be produced using the teachings provided herein. FIG. 2B(i) shows the organization of the LigDFEG gene cluster in a Sphingomonaas paucimobilis strain. FIG. 2B(ii) shows deduced functions of the gene products believed to be involved in a β-aryl ether bond cleavage in a model lignin structure, guaiacylglycerol-β-guaiacyl ether (GGE). The vertical bars above the restriction map indicate the positions of the gene insertions of LigD, LigF, LigE, and LigG.

LigD shoed Cα-dehydrogenase activity, LigF and LigE showed β-etherase activity, and LigG showed glutathione lyase activity. FIG. 2 LEGEND (Abbreviations): restriction enzymes Ap (ApaI), Bs (BstXI), E (EcoRI), Ec (Eco47III), MI (MIuI), P (PstI), RV (EcoRV), S, (SaII), Sc (SacI). ScII (SacII), St (StuI), Sm (SmaI), Tt (TthIIII), and X (XhoI); chemicals GGE (guaiacylglycerol-β-guaiacyl ether), GSH (glutathione), GSSG (glutathione disulfide), and asterisks are asymmetric carbons.

Commercial products that can be obtained from a bioconversion of lignin-derived compounds, as taught herein, include mono-aromatic chemicals. Examples of such chemicals include, but are not limited to, caprolactam, cumene, styrene, mononitro- and dinitrotoluenes and their derivatives, 2,4-diaminotoluene, 2,4-dinitrotoluene, terephthalic acid, catechol, vanillin, salicylic acid, aminosalicylic acid, cresol and isomers, alkylphenols, chlorinated phenols, nitrophenols, polyhydric phenols, nitrobenzene, aniline and secondary and tertiary aniline bases, benzothiazole and derivatives, alkylbenzene and alkylbenzene sulfonates, 4,4-diphenylmethane diisocyanate (MDI), chlorobenzenes and dichlorobenzenes, nitrochlorobenzenes, sulfonic acid derivatives of toluene, pseudocumene, mesitylene, nitrocumene, cumenesulfonic acid.

### Enzyme Discovery

The teachings herein are also directed to the discovery of novel enzymes. In some embodiments, the enzymes are beta-etherase enzymes.

Lignin is the only plant biomass constituent based on aromatic core structures, and is comprised of branched phenylpropenyl (C9) units. The guaiacol and syringol building blocks of lignin are linked through carbon-carbon (C—C) and carbon-oxygen (C—O, ether) bonds. The native structure of lignin suggests its key application as a chemical feedstock for aromatic chemicals. The production of such chemical structures necessitates depolymerization and rupture of C—C and C—O bonds. An abundant chemical linkage in lignin is the beta-aryl ether linkage, which comprises 50% to 70% of the bond type in lignin. The efficient scission of the beta-aryl ether bond would generate the monomeric building blocks of lignin, and provide the chemical feedstock for subsequent conversion to a range of industrial products.

The beta-etherase enzyme system has multiple advantages for conversions of lignin oligomers to monomers over the laccase enzyme systems. The beta-etherase enzyme system would achieve highly selective reductive bond scission catalysis for efficient and high yield conversions of lignin oligomers to monomers without the formation of side products, degradation of the aromatic core structures of lignin, or the use of electron transfer mediators required with use of the oxidative and radical chemistry-based laccase enzyme systems.

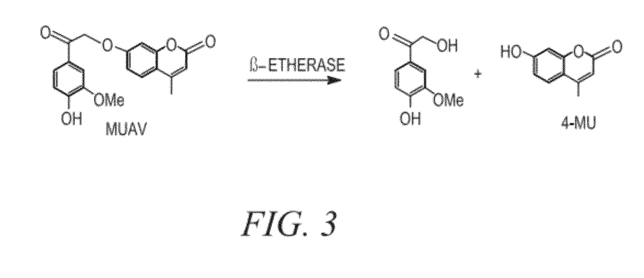

FIG. 3 is an example of a beta-etherase catalyzed hydrolysis of a model lignin dimer, α-O-(β-methylumbelliferyl) acetovanillone (MUAV), according to some embodiments. The scission of the beta-aryl ether bond in model compounds of lignin by beta-etherases from the microbe Sphingmonas paucimobilis has been described. However, the available information is limited, and there is no precedent in the literature for the use of S. paucimobilis as an industrial microbe for commercial scale processes. The discovery of new beta-etherase enzymes, and the heterologous expression of these new enzymes in Azotobacter strains will provide the art with valuable industrial strains that particulary well-suited for lignin conversion processes.

One of skill will recognize the chemical nomenclature used herein as standard to the art. For example, the amino acids used herein can be identified by at least the following conventional three-letter abbreviations in Table 2:

TABLE 2

| | | | | | |
|---|---|---|---|---|---|
| Alanine | A | Ala | Leucine | L | Leu |
| Arginine | R | Arg | Lysine | K | Lys |
| Asparagine | N | Asn | Methionine | M | Met |
| Aspartic acid | D | Asp | Phenylalanine | F | Phe |
| Cysteine | C | Cys | Proline | P | Pro |
| Glutamic acid | E | Glu | Serine | S | Ser |
| Glutamine | Q | Gln | Threonine | T | Thr |
| Glycine | G | Gly | Tryptophan | W | Trp |
| Histidine | H | His | Tyrosine | Y | Tyr |
| Isoleucine | I | Ile | Valine | V | Val |
| Ornithine | O | Orn | Other | | Xaa |

The single letter identifier is provided for ease of reference, but any format can be used. The three-letter abbreviations are generally accepted in the peptide art, recommended by the IUPAC-IUB commission in biochemical nomenclature, and are provided to comply with WIPO Standard ST.25. Furthermore, the peptide sequences are taught according to the generally accepted convention of placing the N-terminus on the left and the C-terminus on the right of the sequence listing to again comply with WIPO Standard ST.25.

The Recombinant Polypeptides

The teachings herein are based on discovery of novel and non-obvious proteins, DNAs, and host cell systems that can function in the conversion of lignin-derived compounds into valuable aromatic compounds. The systems can include natural, wild-type components or recombinant components, the recombinant components being isolatable from what occurs in nature.

The term “isolated” means altered “by the hand of man” from its natural state; i.e., if it occurs in nature, it has been changed or removed from its original environment, or both. For example, a naturally occurring polynucleotide or a polypeptide naturally present in a living animal in its natural state is not “isolated,” but the same polynucleotide or polypeptide separated from the coexisting materials of its natural state is “isolated”, as the term is used herein. For example, with respect to polynucleotides, the term isolated means that it is separated from the chromosome and cell in which it naturally occurs. However, a nucleic acid molecule contained in a clone that is a member of a mixed clone library (e.g., a genomic or cDNA library) and that has not been isolated from other clones of the library (e.g., in the form of a homogeneous solution containing the clone without other members of the library) or a chromosome isolated or removed from a cell or a cell lysate (e.g., a “chromosome spread”, as in a karyotype), is not “isolated” for the purposes of the teachings herein. Moreover, a lone nucleic acid molecule contained in a preparation of mechanically or enzymatically cleaved genomic DNA, where the isolation of the nucleic molecule was not the goal, is also not “isolated” for the purposes of the teachings herein. As part of, or following, an intentional isolation, polynucleotides can be joined to other polynucleotides, for mutagenesis, to form fusion proteins, and for propagation or expression in a host, for instance. Isolated polynucleotides, alone or joined to other polynucleotides such as vectors, can be introduced into host cells, in culture or in whole organisms, after which such DNAs still would be isolated, as the term is used herein, because they would not be in their naturally occurring form or environment. Similarly, the isolated polynucleotides and polypeptides may occur in a composition, such as a media formulation, solutions for introduction of polynucleotides or polypeptides, for example, into cells, compositions or solutions for chemical or enzymatic reactions, for instance, which are not naturally occurring compositions, and, therein remain “isolated” polynucleotides or polypeptides within the meaning of that term as it is used herein.

A “vector,” such as an expression vector, is used to transfer or transmit the DNA of interest into a prokaryotic or eukaryotic host cell, such as a bacteria, yeast, or a higher eukaryotic cell. Vectors can be recombinantly designed to contain a polynucleotide encoding a desired polypeptide. These vectors can include a tag, a cleavage site, or a combination of these elements to facilitate, for example, the process of producing, isolating, and purifying a polypeptide. The DNA of interest can be inserted as the expression component of a vector. Examples of vectors include plasmids, cosmids, viruses, and bacteriophages. If the vector is a virus or bacteriophage, the term vector can include the viral/bacteriophage coat. The term “expression vector” is usually used to describe a DNA construct containing gene encoding an expression product of interest, usually a protein, that is expressed by the machinery of the host cell. This type of vector is frequently a plasmid, but the other forms of expression vectors, such as bacteriophage vectors and viral vectors (e.g., adenoviruses, replication defective retroviruses, and adeno-associated viruses), can be used.

In some embodiments, the polypeptides taught herein can be natural or wildtype, isolated and/or recombinant. In some embodiments, the polynucleotides can be natural or wildtype, isolated and/or recombinant. In some embodiments, the teachings are directed to a vector than can include such a polynucleotide or a host cell transformed by such a vector.

In some embodiments, the polypeptide can be an isolated recombinant polypeptide, comprising an amino acid sequence having at least 95% identity to SEQ ID NO:101. The sequence can conserve residues T19, I20, S21, P22, V24, W25, T27, K28, Y29, A30, H33, K34, G35, F36, D39, I40, V41, P42, G43, G44, F45, G47, I48, E50, R51, T52, G53, G54, K100, A101, N104, V111, G112, M115, F116, P166, W107, Y184, Y187, R188, G191, G192, and F195.

In some embodiments, the polypeptide can be an isolated recombinant polypeptide, comprising SEQ ID NO:101; or conservative substitutions thereof outside of the conserved residues. The conserved residues can include T19, I20, S21, P22, V24, W25, T27, K28, Y29, A30, H33, K34, G35, F36, D39, I40, V41, P42, G43, G44, F45, G47, I48, E50, R51, T52, G53, G54; K100, A101, N104, V111, G112, M115, F116, P166, W107, Y184, Y187, R188, G191, G192, and F195.

In some embodiments, the polypeptide can be an isolated recombinant glutathione S-transferase enzyme, comprising an amino acid sequence having at least 95% identity to SEQ ID NO:101. The amino acid sequence can conserve residues T19, I20, S21, P22, V24, W25, T27, K28, Y29, A30, H33, K34, G35, F36, D39, I40, V41, P42, G43, G44, F45, G47, I48, E50, R51, T52, G53, G54; K100, A101, N104, V111, G112, M115, F116, P166, W107, Y184, Y187, R188, G191, G192, and F195; wherein, the amino acid sequence functions to cleave a beta-aryl ether.

In some embodiments, the polypeptide can be an isolated recombinant glutathione S-transferase enzyme, comprising an amino acid sequence having at least 95% identity to SEQ ID NO:101; wherein, the amino acid sequence functions to cleave a beta-aryl ether.

In some embodiments, the polypeptide can be an isolated recombinant polypeptide, comprising (i) a length ranging from about 279 to about 281 amino acids; (ii) a first amino acid region consisting of residues 19-54 from SEQ ID NO:101, or conservative substitutions thereof outside of conserved residues T19, I20, S21, P22, V24, W25, T27, K28, Y29, A30, H33, K34, G35, F36, D39, I40, V41, P42, G43, G44, F45, G47, I48, E50, R51, T52, G53, and G54; wherein, the first amino acid region can be located in the recombinant polypeptide from about residue 14 to about residue 59; and, (iii) a second amino acid region consisting of residues 98-221 from SEQ ID NO:101, or conservative substitutions thereof outside of conserved residues K100, A101, N104, V111, G112, M115, F116, P166, W107, Y184, Y187, R188, G191, G192, and F195; wherein, the second amino acid region is located in the recombinant polypeptide from about residue 93 to about residue 226.

In some embodiments, the polypeptide can be an isolated recombinant glutathione S-transferase enzyme, comprising (i) a length ranging from about 279 to about 281 amino acids; (ii) a first amino acid region having at least 95% identity to residues 19-54 from SEQ ID NO:101 while conserving residues T19, I20, S21, P22, V24, W25, T27, K28, Y29, A30, H33, K34, G35, F36, D39, I40, V41, P42, G43, G44, F45, G47, I48, E50, R51, T52, G53, and G54; wherein, the first amino acid region is located in the recombinant polypeptide from about residue 14 to about residue 59; and, (iii) a second amino acid region having at least 95% identity to residues 98-221 from SEQ ID NO:101 while conserving residues K100, A101, N104, V111, G112, M115, F116, P166, W107, Y184, Y187, R188, G191, G192, and F195; wherein, the second amino acid region can be located in the recombinant polypeptide from about residue 93 to about residue 226; and, the recombinant glutathione S-transferase enzyme can function to cleave a beta-aryl ether.

In some embodiments, the polypeptide can be an isolated recombinant glutathione S-transferase enzyme, comprising an amino acid sequence having at least 95% identity to SEQ ID NO:541; wherein, the amino acid sequence functions to cleave a beta-aryl ether.

In some embodiments, the polypeptide can be an isolated recombinant polypeptide, comprising (i) a length ranging from about 256 to about 260 amino acids; (ii) a first amino acid region consisting of residues 47-57 from SEQ ID NO:541, or conservative substitutions thereof outside of conserved residues A47, I48, N49, P50, G52, V54, P55, V56, L57; wherein, the first amino acid region is located in the recombinant polypeptide from about residue 45 to about residue 57; (iii) a second amino acid region consisting of 63-76 from SEQ ID NO:541; and, (iv) a third amino acid region consisting of residues 99-230 from SEQ ID NO:541, or conservative substitutions thereof outside of conserved residues R100, Y101, K104, D107, M111, N112, S115, M116, K176, L194, I197, N198, S201, H202, and M206; wherein, the second amino acid region is located in the recombinant polypeptide from about residue 94 to about residue 235.

In some embodiments, the polypeptide can be an isolated recombinant glutathione S-transferase enzyme, comprising (i) a length ranging from about 279 to about 281 amino acids; (ii) a first amino acid region having at least 95% identity to 47-57 from SEQ ID NO:541, or conservative substitutions thereof outside of conserved residues A47, I48, N49, P50, G52, V54, P55, V56, L57; wherein, the first amino acid region can be located in the recombinant polypeptide from about residue 45 to about residue 57; (iii) a second amino acid region consisting of 63-76 from SEQ ID NO:541; and, (iv) a third amino acid region having at least 95% identity to residues 99-230 from SEQ ID NO:541, or conservative substitutions thereof outside of conserved residues R100, Y101, K104, D107, M111, N112, S115, M116, K176, L194, I197, N198, S201, H202, and M206; wherein, the second amino acid region can be located in the recombinant polypeptide from about residue 94 to about residue 235; wherein, the recombinant glutathione S-transferase enzyme functions to cleave a beta-aryl ether.

In some embodiments, an amino acid substitution outside of the conserved residues can be a conservative substitution. And, in many embodiments, the amino acid sequence can function to cleave a beta-aryl ether.

### Methods of Preparing the Recombinant SDF-1 Polynucleotide and Polypeptides

The teachings include a method of preparing the polypeptides described herein, comprising culturing a host cell under conditions suitable to produce the desired polypeptide; and recovering the polypeptide from the host cell culture; wherein, the host cell comprises an exogenously-derived polynucleotide encoding the desired polypeptide. In some embodiments, the host cell is E. Coli. In some embodiments, the host cell can be an Azotobacter strain such as, for example, Azotobacter vinelandii.

Initially, a double-stranded DNA fragment encoding the primary amino acid sequence of recombinant polypeptide can be designed. This DNA fragment can be manipulated to facilitate synthesis, cloning, expression or biochemical manipulation of the expression products. The synthetic gene can be ligated to a suitable cloning vector and then the nucleotide sequence of the cloned gene can be determined and confirmed. The gene can be then amplified using designed primers having specific restriction enzyme sequences introduced at both sides of insert gene, and the gene can be subcloned into a suitable subclone/expression vector. The expression vector bearing the synthetic gene for the mutant can be inserted into a suitable expression host. Thereafter the expression host can be maintained under conditions suitable for production of the gene product and, in some embodiments, the protein can be (i) isolated and purified from the cells expressing the gene or (ii) used directly in a reaction environment that includes the host cell.

The nucleic acid (e.g., cDNA or genomic DNA) may be inserted into a replicable vector for cloning (amplification of the DNA) for expression. Various vectors are publicly available. In general, DNA can be inserted into an appropriate restriction endonuclease site(s) using techniques known in the art, for example. Vector components generally include, but are not limited to, one or more of a signal sequence, an origin of replication, one or more marker genes, an enhancer element, a promoter, and a transcription termination sequence.

The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces alpha-factor leaders, the latter described in U.S. Pat. No. 5,010,182), or acid phosphatase leader, the C. albicans glucoamylase leader (EP 362,179), or the signal described in WO 90/13646, for example. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from a plasmid, e.g. pBR322, for example, is suitable for most Gram-negative bacteria, and the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take the encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the encoding nucleic acid sequence to direct mRNA synthesis. Promoters recognized by a variety of potential host cells are well known. Promoters suitable for use with prokaryotic hosts include the .beta.-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21 25 (1983)). Promoters for use in bacterial systems also will contain a Shine-Dalgarno sequence operably linked to the encoding DNA.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are known in the art, e.g. see EP 73,657 for a further discussion.

PRO87299 transcription from vectors in mammalian host cells is controlled, for example, by promoters obtained from the genomes of viruses such as polyoma virus, fowlpox virus (UK 2,211,504), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of the encoding DNA by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the vector at a position 5′ or 3′ to the coding sequence but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the mutants.

In some embodiments, the expression control sequence can be selected from a group consisting of a lac system, T7 expression system, major operator and promoter regions of pBR322 origin, and other prokaryotic control regions. Still other methods, vectors, and host cells suitable for adaptation to the synthesis of the mutants in recombinant vertebrate cell culture are described in Gething et al., Nature, 293:620 625 (1981); Mantei et al., Nature, 281:40 46 (1979); EP 117,060; and EP 117,058.

Mutants can be expressed as a fusion protein. In some embodiments, the methods involve adding a number of amino acids to the protein, and in some embodiments, to the amino terminus of the protein. Extra amino acids can serve as affinity tags or cleavage sites, for example. Fusion proteins can be designed to: (1) assist in purification by acting as a temporary ligand for affinity purification, (2) produce a precise recombinant by removing extra amino acids using a cleavage site between the target gene and affinity tag, (3) increase the solubility of the product, and/or (4) increase expression of the product. A proteolytic cleavage site can be included at the junction of the fusion region and the protein of interest to enable further purification of the product—separation of the recombinant protein from the fusion protein following affinity purification of the fusion protein. Such enzymes, and their cognate recognition sequences, can include Factor Xa, thrombin and enterokinase, cyanogen bromide, trypsin, or chymotrypsin, for example. Typical fusion expression vectors include pGEX (Pharmacia Biotech Inc; Smith, D. B. and Johnson, K. S. Gene 67:31-40 (1988)), pMAL (New England Biolabs, Beverly, Mass.), pRIT5 (Pharmacia, Piscataway, N.J.), and pET (Strategen), which can fuse glutathione S-transferase (GST), maltose E binding protein, protein A, or a six-histidine sequence, respectively, to a target recombinant protein.

Synthetic DNAs containing the sequences of nucleotides, tags and cleavage sites can be designed and provided as a modified coding for recombinant polypeptide mutants. In some embodiments, a polypeptide can be a fusion polypeptide having an affinity tag, and the recovering step includes (1) capturing and purifying the fusion polypeptide, and (2) removing the affinity tag for high yield production of the desired polypeptide or an amino acid sequence that is at least 95% homologous to a desired polypeptide. DNA encoding the mutants may be obtained from a cDNA library prepared from tissue possessing the mRNA for the mutants. As such, the DNA can be conveniently obtained from a cDNA library. The encoding gene for the mutants may also be obtained from a genomic library or by known synthetic procedures (e.g., automated nucleic acid synthesis).

Libraries can be screened with probes designed to identify the gene of interest or the protein encoded by it. Screening the cDNA or genomic library with the selected probe may be conducted using standard hybridization procedures, such as described in Sambrook et al., Molecular Cloning: A Laboratory Manual (New York: Cold Spring Harbor Laboratory Press, 1989), which is herein incorporated by reference. An alternative means to isolate the gene encoding recombinant polypeptide mutants is to use PCR methodology [Sambrook et al., supra; Dieffenbach et al., PCR Primer: A Laboratory Manual (Cold Spring Harbor Laboratory Press, 1995)].

Nucleic acids having a desired protein coding sequence may be obtained by screening selected cDNA or genomic libraries using a deduced amino acid sequence and, if necessary, a conventional primer extension procedure as described in Sambrook et al., supra, to detect precursors and processing intermediates of mRNA that may not have been reverse-transcribed into cDNA.

The selection of expression vectors, control sequences, transformation methods, and the like, are dependent on the type of host cell used to express the gene. Following entry into a cell, all or part of the vector DNA, including the insert DNA, may be incorporated into the host cell chromosome, or the vector may be maintained extrachromosomally. Those vectors that are maintained extrachromosomally are frequently capable of autonomous replication in the host cell. Other vectors are integrated into the genome of a host cell upon and are replicated along with the host genome.

Host cells are transfected or transformed with the expression or cloning vectors described herein to produce the mutants. The cells are cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra, each of which are incorporated by reference.

The host cells can be prokaryotic or eukaryotic and, suitable host cells for cloning or expressing the DNA in the vectors herein can include prokaryote, yeast, or higher eukaryote cells. Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456 457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527 537 (1990) and Mansour et al., Nature, 336:348 352 (1988).

Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimunrium, Serratia, e.g., Serratia marcescans, and Shigella, as well as Bacilli such as B. subtilis and B. lichenifomis (e.g., B. lichenifomis 41 P disclosed in DD 266,710 published 12 Apr. 1989), Pseudomonas such as P. aeruginosa, and Streptomyces. These examples are illustrative rather than limiting, and merely supplement the remainder of the teachings herein. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1 A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvC kanr; E. coli W3110 strain 40B4, which is 37D6 with a non-kanamycin resistant degP deletion mutation; and an E. coli strain having mutant periplasmic protease as disclosed in U.S. Pat. No. 4,946,783. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for the mutants. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9:968 975 (1991)) such as, e.g., K. lactis (MW98-8C, CBS683, CBS4574; Louvencourt et al., J. Bacteriol., 154(2):737 742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265 278 [1988]); Candida; Trichoderma reesia (EP 244,234); Neurospora crassa (Case et al., Proc. Natl. Acad. Sci. USA, 76:5259 5263 (1979)); Schwanniomyces such as Schwanniomyces occidentalis (EP 394,538); and filamentous fungi such as, e.g., Neurospora, Penicillium, Tolypocladium (WO 91/00357), and Aspergillus hosts such as A. nidulans (Ballance et al., Biochem. Biophys. Res. Commun., 112:284 289 (1983); Tilburn et al., Gene, 26:205 221 (1983); Yelton et al., Proc. Natl. Acad. Sci. USA, 81: 1470 1474 (1984)) and A. niger (Kelly and Hynes, EMBO J., 4:475 479 (1985)) Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated mutants can be derived from multicellular organisms. Invertebrate cells include insect cells such as Drosophila S2 and Spodoptera Sf9, as well as plant cells. Useful mammalian host cell lines include Chinese hamster ovary (CHO) and COS cells. More specific examples include monkey kidney CVI line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR(CHO, Urlaub and Chasin, Proc. Natl. Acad. Sci. USA, 77:4216 (1980)); mouse sertoli cells (TM4, Mather, Biol. Reprod., 23:243 251 (1980)); human lung cells (W138, ATCC CCL 75); human liver cells (Hep G2, HB 8065); and mouse mammary tumor (MMT 060562, ATCC CCL5 1). One of skill can readily choose the appropriate host cell, at least for extracellular protein harvesting embodiments, without undue experimentation.

In some embodiments, a nucleotide sequence will be hybridizable, under moderately stringent conditions, to a nucleic acid having a nucleotide sequence comprising or complementary to the desired nucleotide sequences. In some embodiments, an isolated nucleotide sequence will be hybridizable, under stringent conditions, to a nucleic acid having a nucleotide sequence comprising or complementary to the desired nucleotide sequences. A nucleic acid molecule can be “hybridizable” to another nucleic acid molecule when a single-stranded form of the nucleic acid molecule can anneal to the other nucleic acid molecule under the appropriate conditions of temperature and ionic strength (see Sambrook et al., supra,). The conditions of temperature and ionic strength determine the “stringency” of the hybridization. “Hybridization” requires that two nucleic acids contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

In some embodiments, the polynucleotides and polypeptides have at least 55, 60, 65, 70, 75, 80, 85, 90, 91, 92, 93, 94, 95, 96, 97, 98, or 99 percent homology to a desired polynucleotide or polypeptide. In some embodiments, the polynucleotides and polypeptides have at least 55, 60, 65, 70, 75, 80, 85, 90, 91, 92, 93, 94, 95, 96, 97, 98, or 99 percent identity to a desired polynucleotide or polypeptide. And, in some embodiments, the polynucleotides and polypeptides have at least 55, 60, 65, 70, 75, 80, 85, 90, 91, 92, 93, 94, 95, 96, 97, 98, or 99 percent similarity to a desired polynucleotide or polypeptide. As described above, degenerate forms of the desired polynucleotide are also acceptable. In some embodiments, a polypeptide can be 90, 91, 92, 93, 94, 95, 96, 97, 98, or 99 homologous, identical, or similar to a desired polypeptide as long as it shares the same function as the desired polypeptide, and the extent of the function can be less or more than that of the desired polypeptide. In some embodiments, for example, a polypeptide can have a function that is 10%, 20%, 30%, 40%, 50%, 60%, 70%, 80%, 90%, 100%, or any 0.1% increment in-between, that of the desired polypeptide. And, in some embodiments, for example, a polypeptide can have a function that is 110%, 120%, 130%, 140%, 150%, 160%, 170%, 180%, 190%, 200%, 300%, 400%, 500%, or more, or any 1% increment in-between, that of the desired polypeptide. In some embodiments the “function” is an enzymatic activity, measurable by any method known to one of skill such as, for example, a method used in the teachings herein. The “desired polypeptide” or “desired polynucleotide” can be referred to as a “reference polypeptide” or “reference polynucleotide”, or the like, in some embodiments as a control for comparison of a polypeptide of interest, which may be considered a “test polypeptide” or “test polynucleotide” or the like. In any event, the comparison is that of one set of bases or amino acids against another set for purposes of measuring homology, identity, or similarity. The ability to hybridize is, of course, another way of comparing nucleotide sequences.

The terms “homology” and “homologous” can be used interchangeably in some embodiments. The terms can refer to nucleic acid sequence matching and the degree to which changes in the nucleotide bases between polynucleotide sequences affects the gene expression. These terms also refer to modifications, such as deletion or insertion of one or more nucleotides, and the effects of those modifications on the functional properties of the resulting polynucleotide relative to the unmodified polynucleotide. Likewise the terms refer to polypeptide sequence matching and the degree to which changes in the polypeptide sequences, such as those seen when comparing the modified polypeptides to the unmodified polypeptide, affect the function of the polypeptide. It should appreciated to one of skill that the polypeptides, such as the mutants taught herein, can be produced from two non-homologous polynucleotide sequences within the limits of degeneracy.

The terms “similarity” and “identity” are known in the art. The term “identity” can be used to refer to a sequence comparison based on identical matches between correspondingly identical positions in the sequences being compared. The term “similarity” can be used to refer to a comparison between amino acid sequences, and takes into account not only identical amino acids in corresponding positions, but also functionally similar amino acids in corresponding positions. Thus similarity between polypeptide sequences indicates functional similarity, in addition to sequence similarity. Levels of identity between gene sequences and levels of identity or similarity between amino acid sequences can be calculated using known methods. For example, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis. In some embodiments, the Gap program, with a Gap penalty of 12 and a Gap length penalty of 4 can be used for determining the amino acid sequence comparisons, and a Gap penalty of 50 and a Gap length penalty of 3 for the polynucleotide sequence comparisons. In some embodiments, the sequences can be aligned so that the highest order match is obtained. The match can be calculated using published techniques that include, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991, each of which is incorporated by reference herein.

As such, the term “similarity” is similar to “identity”, but in contrast to identity, similarity can be used to refer to both identical matches and conservative substitution matches. For example, if two polypeptide sequences have 10/20 identical amino acids, and the remainder are all non-conservative substitutions, then the percent identity and similarity would both be 50%. On the other hand, if there are 5 five more positions where there are conservative substitutions, then the percent identity is 50%, whereas the percent similarity is 75%.

In some embodiments, the term “substantial sequence identity” can refer to an optimal alignment, such as by the programs GAP or BESTFIT using default gap penalties, having at least 65, 70, 75, 80, 85, 90, 95, 96, 97, 98, or 99 percent sequence identity. The difference in what is “substantial” regarding identity can often vary according to a corresponding percent similarity, since the factor of primary importance is often the function of the sequence in a system. The term “substantial percent identity” can be used to refer to a DNA sequence that is sufficiently similar to a reference sequence at the nucleotide level to code for the same protein, or a protein having substantially the same function, in which the comparison can allow for allelic differences in the coding region. Likewise, the term can be used to refer to a comparison of sequences of two polypeptides optimally aligned.

In some embodiments, sequence comparisons can be made to a reference sequence over a “comparison window” of amino acids or bases that includes any number of amino acids or bases that is useful in the particular comparison. For example, the reference sequence may be a subset of a larger sequence. In some embodiments, the comparison window can include at least 10 residue or base positions, and sometimes at least 15-20 amino acids or bases. The reference or test sequence may represent, for example, a polypeptide or polynucleotide having one or more deletions, substitutions or additions.

The term “variant” refers to modifications to a peptide that allows the peptide to retain its binding properties, and such modifications include, but are not limited to, conservative substitutions in which one or more amino acids are substituted for other amino acids; deletion or addition of amino acids that have minimal influence on the binding properties or secondary structure; conjugation of a linker; post-translational modifications such as, for example, the addition of functional groups. Examples of such post-translational modifications can include, but are not limited to, the addition of modifying groups described below through processes such as, for example, glycosylation, acetylation, phosphorylation, modifications with fatty acids, formation of disulfide bonds between peptides, biotinylation, PEGylation, and combinations thereof. In fact, in most embodiments, the polypeptides can be modified with any of the various modifying groups known to one of skill.

The terms “conservatively modified variant,” “conservatively modified substitution,” and “conservative substitution” can be used interchangeably in some embodiments. These terms can be used to refer to a conservative amino acid substitution, which is an amino acid substituted by an amino acid of similar charge density, hydrophilicity/hydrophobicity, size, and/or configuration such as, for example, substituting valine for isoleucine. In comparison, a “non-conservatively modified variant” refers to a non-conservative amino acid substitution, which is an amino acid substituted by an amino acid of differing charge density, hydrophilicity/hydrophobicity, size, and/or configuration such as, for example, substituting valine for phenyalanine. One of skill will appreciate that there are a plurality of ways to define conservative substitutions, and any of these methods may be used with the teachings provided herein. In some embodiments, for example, a substitution can be considered conservative if an amino acid falling into one of the following groups is substituted by an amino acid falling in the same group: hydrophilic (Ala, Pro, Gly, Glu, Asp, Gln, Asn, Ser, Thr), aliphatic (Val, Ile, Leu, Met), basic (Lys, Arg, H is), aromatic (Phe, Tyr, Trp), and sulphydryl (Cys). See Dayhoff, M O. Et al. National Biomedical Research Foundation, Georgetown University, Washington D.C.:89-99 (1972), which is incorporated herein. In some embodiments, the substitution of amino acids can be considered conservative where the side chain of the substitution has similar biochemical properties to the side chain of the substituted amino acid.

### Microbial Systems—Antimicrobial Lignin-Derived Compounds

The antimicrobial activity of lignin-derived compounds is a major problem addressed by the systems taught herein. For example, typical industrial fermentation processes might utilize the microbes Escherichia coli K12 or Escherichia coli B, or the yeast Saccharomyces cerevisiae, and recombinant versions of these microbes, which are well characterized industrial strains. The problem is that the antimicrobial activities of aromatic compounds on such industrial microbes are toxic to the microbes, which negates an application to biotransformations of lignin-derived compounds.

The phenolic streams or soluble lignin streams derived from pretreated lignocellulosic biomass, for example, might contain aromatic and nonaromatic compounds, such as gallic acid, hydroxymethylfurfural alcohol, hydroxymethylfurfural, furfural alcohol, 3,5-dihydroxybenzoate, furoic acid, 3,4-dihydroxybenzaldehyde, hydroxybenzoate, homovanillin, syringic acid, vanillin, and syringaldehyde. There are several lignin-derived compounds that are antimicrobials. For example, furfural, 4-hydroxybenzaldehyde, syringaldehyde, 5-hydroxymethylfurfural, and vanillin are each known to have antimicrobial activity against Escherichia coli, and might have an additive antimicrobial activity against Escherichia coli when present in combination. Moreover, veratraldehyde, cinnamic acid and the respective benzoic acid derivatives of vanillic acid, vanillylacetone, and the cinnamic acid derivatives o-coumaric acid, m-coumaric acid, and p-coumaric acid might be components of the phenolic streams from pretreated lignocellulosic biomass. Veratraldehyde, cinnamic acid and the respective benzoic acid derivatives of vanillic acid, vanillylacetone, and cinnamic acid derivatives o-coumaric acid, m-coumaric acid, and p-coumaric acid, each have significant antifungal activities against the yeast Saccharomyces cerevisiae, and might have an additive antifungal activity against the yeast Saccharomyces cerevisiae when present in combination.

One or more of the following benzaldehyde derivatives might be present in the phenolic streams from pretreated lignocellulosic biomass: 2,4,6-trihydroxybenzaldehyde, 2,5-dihydroxybenzaldehyde, 2,3,4-trihydroxybenzaldehyde, 2-hydroxy-5-methoxybenzaldehyde, 2,3-dihydroxybenzaldehyde, 2-hydroxy-3-methoxybenzaldehyde, 4-hydroxy-2,6-dimethoxybenzaldehyde, 2,5-dihydroxybenzaldehyde, 2,4-dihydroxybenzaldehyde, and 2-hydroxybenzaldehyde. Likewise, 2,4,6-trihydroxybenzaldehyde, 2,5-dihydroxybenzaldehyde, 2,3,4-trihydroxybenzaldehyde, 2-hydroxy-5-methoxybenzaldehyde, 2,3-dihydroxybenzaldehyde, 2-hydroxy-3-methoxybenzaldehyde, 4-hydroxy-2,6-dimethoxybenzaldehyde, 2,5-dihydroxybenzaldehyde, 2,4-dihydroxybenzaldehyde, and 2-hydroxybenzaldehyde have each demonstrated antibacterial activity against Escherichia coli, and might have an additive antibacterial activity against Escherichia coli when present in combination.

### Microbial Systems—Suitable Microbes

The antimicrobial activity of lignin-derived compounds creates a need for a strain of microbe that is tolerant to such activity in the reaction environment. The teachings include the identification of recombinant or non-recombinant microbial species that are naturally capable of metabolizing aromatic compounds for the biotransformations of lignin-derived compounds to commercial products.

Some examples of microbial species particularly suited for biotransformations of phenolic streams from pretreated lignocellulosic biomass include, but are not limited to, Azotobacter chroococcum, Azotobacter vinelandii, Novosphingobium aromaticivorans, Pseudomonas aeruginosa, Pseudomonas putida, Pseudomonas fluorescens, Pseudomonas stutzerii, Pseudomonas diminuta, Pseudomonas pseudoalcaligenes, Rhodopseudomonas palustris, Spingomonas sp.A1, Sphingomonas paucimobilis SYK-6, Sphingomonas japonicum, Sphingomonas alaskenesis, Sphingomonas wittichii, Streptomyces viridosporus, Delftia acidivorans, and Rhodococcus equi. Both bio-informatic and experimental data from the literature reveal the presence of extensive metabolic activity towards aromatic compounds in these strains, making them relevant species for the discovery of enzymes that hydrolyze lignin-derived oligomers, and for biotransformations of lignin core structures. Without intending to be bound by any theory or mechanism of action, these species exhibit, for example, metabolism of aromatic compounds such as benzoate; amino-, fluoro-, and chloro-benzoates; biphenyl; toluene and nitrotoluenes; xylenes; alkylbenzenes; styrene; atrazine; caprolactam; and polycyclic aromatic hydrocarbons.

The microbes can be grown in a fermentor, for example, using methods known to one of skill. The enzymes used in the bioprocessing are obtained from the microbes, and they can be intracellular, extracellular, or a combination thereof. As such, the enzymes can be recovered from the host cells using methods known to one of skill in the art that include, for example, filtering or centrifuging, evaporation, and purification. In some embodiments, the method can include breaking open the host cells using ultrasound or a mechanical device, remove debris and extract the protein, after which the protein can be purified using, for example, electrophoresis. In some embodiments, however, the teachings include the use of a microbe, recombinant or non-recombinant, that has tolerance to lignin-derived compounds. A microbe that is tolerant to lignin-derived compounds can be used industrially, for example, to express any enzyme, recombinant or non-recombinant, having a desired enzyme activity while directly in association with the lignin-derived compounds. Such activities include, for example, beta etherase activity, C-alpha-dehydrogenase activity, glutathione lyase activity, or any other enzyme activity that would be useful in the biotransformation of lignin-derived compounds. The activities can be wild-type or produce through methods known to one of skill, such as transfection or transformation, for example.

### Microbial Systems—Azotobacter Strains

The teachings herein are also directed to the discovery and use of recombinant Azotobacter strains heterologously expressing novel beta-etherase enzymes for the hydrolysis of lignin oligomers.

Research directed to the discovery of a suitable microbe has shown that Azotobacter vinelandii may possess the industrially relevant strain criteria desired for the teachings provided herein. In some embodiments, the criteria includes (i) growth on inexpensive and defined medium, (ii) resistance to inhibitors in hydrolysates of lignocellulose, (iii) tolerance to acidic pH and higher temperatures, (iv) the co-fermentation of pentose and hexose sugars, (v) genetic tractability and availability of gene expression tools, (vi) rapid generation times, and (vii) successful growth performance in pilot scale fermentations. Additionally, key physiological traits that contribute to the potential suitability of A. vinelandii to the conversion of lignin-streams include an ability to metabolize aromatic compounds and xenobiotics. Moreover, it has been shown to have a tolerance to phenolic compounds in industrial waste streams. The annotated genome sequence of A. vinelandii, and the availability of genetic tools for its transformation and for the heterologous expression of enzymes, contribute to the potential of this microbe to function, in it's native form or as a transformant, for example, in a high-yield production of industrial chemicals from lignin streams.

The teachings are also directed to a method of cleaving a beta-aryl ether bond, the comprising contacting a polypeptide taught herein with a lignin-derived compound having (i) a beta-aryl ether bond and (ii) a molecular weight ranging from about 180 Daltons to about 3000 Daltons; wherein, the contacting occurs in a solvent environment in which the lignin-derived compound is soluble. The term “contacting” refers to placing an agent, such as a compound taught herein, with a target compound, and this placing can occur in situ or in vitro, for example.

The teachings are also directed to a method of cleaving a beta-aryl ether bond, the comprising contacting a polypeptide taught herein with a lignin-derived compound having (i) a beta-aryl ether bond and (ii) a molecular weight ranging from about 180 Daltons to about 3000 Daltons; wherein, the contacting occurs in a solvent environment in which the lignin-derived compound is soluble. In some embodiments, the lignin-derived compound has a molecular weight of about 180 Daltons to about 1000 Daltons. In some embodiments, the solvent environment comprises water. And, in some embodiments, the solvent environment comprises a polar organic solvent.

The teachings are also directed to a system for bioprocessing lignin-derived compounds, the system comprising a polypeptide taught herein, a lignin-derived compound having a beta-aryl ether bond and a molecular weight ranging from about 180 Daltons to about 3000 Daltons; and, a solvent in which the lignin-derived compound is soluble; wherein, the system functions to cleave the beta-aryl ether bond by contacting the polypeptide with the lignin-derived compound in the solvent.

The teachings are also directed to a recombinant polynucleotide comprising a nucleotide sequence that encodes a polypeptide taught herein. Likewise, the teachings are also directed to a vector or plasmid comprising the polynucleotide, as well as a host cell transformed by the vector or plasmid to express the polypeptide.

The teachings are also directed to a method of cleaving a beta-aryl ether bond, the method comprising (i) culturing a host cell taught herein under conditions suitable to produce a polypeptide taught herein; (ii) recovering the polypeptide from the host cell culture; and, (iii) contacting the polypeptide of claim 1 with a lignin-derived compound having a beta-aryl ether bond and a molecular weight ranging from about 180 Daltons to about 3000 Daltons; wherein, the contacting occurs in a solvent environment in which the lignin-derived compound is soluble.

In some embodiments, the host cell can be E. Coli or an Azotobacter strain, such as Azotobacter vinelandii. And, in some embodiments, the lignin-derived compound can have a molecular weight of about 180 Daltons to about 1000 Daltons.

The teachings are also directed to a system for bioprocessing lignin-derived compounds, the system comprising (i) a transformed host cell taught herein; (ii) a lignin-derived compound having a beta-aryl ether bond and a molecular weight ranging from about 180 Daltons to about 3000 Daltons; and, (iii) a solvent in which the lignin-derived compound is soluble; wherein, the system functions to cleave the beta-aryl ether bond by contacting a polypeptide taught herein with the lignin-derived compound in the solvent.

### EXAMPLES

The following examples illustrate, but do not limit, the present invention.

### Example 1

Microbial growth and metabolism studies on soluble lignin samples are performed to test the tolerance of microbes on lignin-derived compounds. A set of aromatic and nonaromatic compounds known to inhibit growth of E. coli and S. cerevisiae strains might be used to characterize the growth, tolerance and metabolic capability of Azotobacter vinelandii strain BAA1303, and A. chroococcum strain 4412 (EB Fred) X-50. Metabolism of various aromatic and nonaromatic compounds by microbial strains might be determined as a function of cellular respiration by the reduction of soluble tetrazolium salts by actively metabolizing cells. XTT (2,3-Bis(2-methoxy-4-nitro-5-sulfophenyl)-2H-tetrazolium-5-carboxanilide inner salt, Sigma) is reduced to a soluble purple formazan compound by respiring cells. E. coli might be used as the negative control strain in this study. Strains might be grown in rich medium to saturation, washed, and OD600 nm of the cultures determined. Equal numbers of bacteria will be inoculated into wells of the 48-well growth asing concentrations of aromatic and non-aromatic compounds in the range of 0-500 mM, will be added to the wells to a final volume of 0.8 ml. Following incubation for 24-48 hours with shaking at 25-37° C., the cultures will be tested for growth upon exposure to the test compounds using the XTT assay kit (Sigma). Culture samples will removed from the 48 well growth plate, and diluted appropriately in 96 well assay plates to which the XTT reagent will be added. Soluble formazan formed will be quantified by absorbance at 450 nm. Increased absorbance at 450 nm will be indicative of growth or survival, or metabolism of a particular test compound by the strains. Table 3 lists some example compounds that can be used to test the tolerance of microbes on lignin-derived compounds.

TABLE 3

| | Test Compound |
|---|---|
| 1 | Syringic acid |
| 2 | Syringaldehyde |
| 3 | Gallic acid |
| 4 | Furfural |
| 5 | 5-Hydroxymethylfurfural |
| 6 | 4-hydroxybenzaldehyde |
| 7 | Hydroxybenzoate |
| 8 | Vanillin |
| 9 | Vanillic acid |
| 10 | Cinnamic acid |
| 11 | o-, m-and p-Coumaric acids |
| 12 | 2-hydroxy-3-methoxybenzaldehyde |
| 13 | 2,4,6-trihydroxybenzaldehyde |
| 14 | 4-hydroxy-2,6-dimethoxybenzaldehyde |

The set of lignin compounds to be tested might be expanded to any of the teachings provided herein. And, the microbial growth and metabolism studies on soluble lignin samples can also be performed actual industrial samples such as, for example, kraft lignins and biorefinery lignins.

### Example 2

This example illustrates how prospective enzymes were identified for use with the teachings provided herein. Although never successfully expressed heterologously as an industrial microbe in a commercial scale process, Sphingomonas paucimobilis has been shown to produce enzymes that have some activity in cleaving the beta aryl ether bond in lignin. See Masai, E., et al. Accordingly, the enzyme discovery effort started with running BLAST searches against the two enzymes identified by Masai as having beta etherase activity, “ligE” and “ligF”. See Id. at Abstract. Table 4 lists genes identified in the BLAST searches for initial screening.

TABLE 4

| | | | | | Identity/ |
|---|---|---|---|---|---|
| | | | | Genbank | Similarity |
| | Gene | Species | Activity | Accession # | (%) |
| 1 | ligE | Sphingomonas | Beta- | BAA02032.1 | |
| | | paucimobilis | etherase | | |
| 2 | ligE-1 | Novosphingobium | Putative | ABD26841.1 | (62%) (75%) |
| | | aromaticivorans | Beta- | | |
| | | | etherase | | |
| 3 | ligF | Sphingomonas | Beta- | BAA02031.1 | |
| | | paucimobilis | etherase | | |
| 4 | ligF-1 | Novosphingobium | Putative | ABD26530.1 | (60%) (77%) |
| | | aromaticivorans | Beta- | | |
| | | | etherase | | |
| 5 | ligF-2 | Novosphingobium | Putative | ABD27301.1 | (47%) (59%) |
| | | aromaticivorans | Beta- | | |
| | | | etherase | | |
| 6 | ligF-3 | Novosphingobium | Putative | ABD27309.1 | (37%) (57%) |
| | | aromaticivorans | Beta- | | |
| | | | etherase | | |

The nucleotide and amino acid sequences in Table 4 are incorporated herein by reference in their entirety through the GenBank Accession Numbers.

### Example 3

This example describes a method for preparing recombinant host cells for the heterologous expression of known and putative beta-etherase encoding gene sequences in Escherichia coli (E. coli). E. coli is used in this example as a surrogate enzyme production host organism for the enzyme discovery. The construction of a novel industrial host microbe, A. vinelandii is described below.

The gene sequences with accession numbers in Table 3 were synthesized directly as open reading frames (ORFS) from oligonucleotides by using standard PCR-based assembly methods, and using the E. coli codon bias with 10% threshold. The end sequences contained adaptors (NdeI and XhoI) for restriction digestion and cloning into the E. coli expression vector pET24b (Novagen). Internal NdeI and XhoI sites were excluded from the ORF sequences during design of the oligonucleotides. Assembled genes were cloned into a cloning vector (pGOV4), transformed into E. coli CH3 chemically competent cells, and DNA sequences determined from purified plasmid DNA. After sequence verification, restriction digestion was used to excise each ORF fragment from the cloning vector, and the sequence sub-cloned into pET24b. The entire set of ligE and ligF bearing plasmids were then transformed into E. coli BL21 (DE3) which served as the host strain for beta-etherase expression and biochemical activity testing.

LigE, from Accession No BAA2032.1, is listed herein as SEQ ID NO:1 for the protein and SEQ ID NO:2 for the gene. An “optimized” nucleic acid sequence was created to facilitate the transformation in E. coli and is listed herein as SEQ ID NO:977.

LigE-1, from Accession No ABD26841.1, is listed herein as SEQ ID NO:101 for the protein and SEQ ID NO:102 for the gene. An “optimized” nucleic acid sequence was created to facilitate the transformation in E. coli and is listed herein as SEQ ID NO:978.

LigF, from Accession No BAA2031.1 (P30347.1), is listed herein as SEQ ID NO:513 for the protein and SEQ ID NO:514 for the gene. An “optimized” nucleic acid sequence was created to facilitate the transformation in E. coli and is listed herein as SEQ ID NO:979.

LigF-1, from Accession No ABD26530.1, is listed herein as SEQ ID NO:539 for the protein and SEQ ID NO:540 for the gene. An “optimized” nucleic acid sequence was created to facilitate the transformation in E. coli and is listed herein as SEQ ID NO:980.

LigF-2, from Accession No ABD27301.1, is listed herein as SEQ ID NO:541 for the protein and SEQ ID NO:542 for the gene. An “optimized” nucleic acid sequence was created to facilitate the transformation in E. coli and is listed herein as SEQ ID NO:981.

LigF-3, from Accession No ABD27309.1, is listed herein as SEQ ID NO:545 for the protein and SEQ ID NO:546 for the gene. An “optimized” nucleic acid sequence was created to facilitate the transformation in E. coli and is listed herein as SEQ ID NO:982.

### Example 3

This example describes a method for gene expression in E. coli, as well as beta-etherase biochemical assays. Expression of known and putative beta-etherase genes was performed using 5 ml cultures of the recombinant E. coli strains described herein in Luria Broth medium by induction of gene expression using isopropylthiogalactoside (IPTG) to a final concentration of 0.1 mM. Following induction, and cell harvest, the cells were disrupted using either sonication or the BPER (Invitrogen) cell lysis system.

Clarified cell extracts were tested in the in vitro biochemical assay for beta-etherase activity on a fluorescent substrate, a model lignin dimer compound α-O-(β-methylumbelliferyl) acetovanillone (MUAV). In vitro reactions were performed in a total volume of 200 ul and contained: 25 mM TrisHCl pH 7.5; 0.5 mM dithiothreitol; 1 mM glutathione; 0.05 mM or 0.1 mM MUAV; 10 ul of clarified cell extract used to initiate the reactions. Following incubation for 2.5 hours at room temperature, a 50 ul sample of the reactions was terminated using 150 uL of 300 mM glycine/NaOH buffer pH 9. The formation of 4 methylumbelliferone (4MU) upon hydrolysis of the aryl ether bond was monitored by the increase in fluorescence at λₑₓ=360 nm and λₑₘ=450 nm using a Spectramax UV/visible/fluorescent spectrophotometer.

The total protein concentrations of the cell lysates were determined using the BCA reagent system for protein quantification (Pierce).

Induction might be also performed using IPTG concentrations in the range of 0.01-1 mM. Cell disruption might be also performed using toluene permeabilization, French pressure techniques, or using multiple freeze/thaw cycles in conjunction with lysozyme. Assay conditions might be varied to include TrisHCl at 10-150 mM concentrations and in the pH range of 6.5-8.5; 0-2 mM dithiothreitol; 0.05-2 mM glutathione; 0.01-5 mM MUAV substrate; 22-42° C. reaction temperatures. The biochemical assay might be performed as a fixed time point assay with reaction times ranging from 5 minutes-12 hours, or performed continuously without quenching with glycine/NaOH buffer to extract enzyme kinetic parameters.

### Example 4

This example describes the tested biochemical activities of the newly-discovered beta-etherase enzymes.

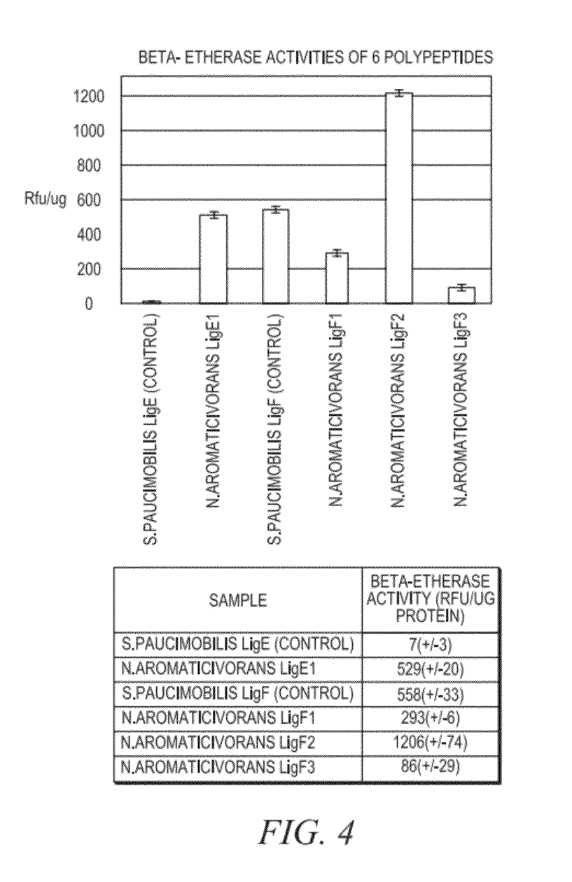

FIG. 4 illustrates unexpected results from biochemical activity assays for beta-etherase function for the S. paucimobilis positive control polypeptides, and the N. aromaticivorans putative beta-etherase polypeptide, according to some embodiments. The much elevated beta-etherase activity exhibited by the putative ligE1 gene product from N. aromaticivorans as compared to the S. paucimobilis ligE gene product was a completely unexpected result of the enzyme discovery program.

In reactions containing 0.1 mM MUAV substrate, E. coli cell extracts expressing the N. aromaticovorans ligE1 protein yielded a total activity of 529 rfu/ug compared to 7 rfu/ug for the S. paucimobilis ligE protein. The newly discovered beta-etherase from N. aromaticovorans is approximately 75-fold more efficient than the previously described S. paucimobilis ligE beta-etherase enzyme. The highly efficient novel beta-etherase is ideally suited to be a biocatalyst for conversion of lignin aryl ethers to monomers in biotechnological processes.

It was also surprising to find that 3 novel N. aromaticivorans polypeptides having identities to the S. paucimobilis LigF sequence showed beta-etherase activity on the MUAV substrate. While all 3 putative ligF gene products from N. aromaticivorans exhibited beta-etherase activity, the LigF2 polypeptide is approximately 2-fold more efficient than the S. paucimobilis LigF protein. The N. aromaticovorans LigF2 protein yielded a total activity of 1206 rfu/ug compared to 558 rfu/ug for the S. paucimobilis LigF protein.

As such, the enzyme discovery program unexpectedly and surprisingly generated four (4) novel polypeptides from N. aromaticivorans with beta-etherase activity. This set of enzymes show great potential for the catalysis of a complete depolymerization of lignin-derived compounds. The results were unexpected and surprising for at least the following reasons:

Four (4) novel gene sequences encoding polypeptides with beta-etherase activity were discovered from N. aromaticivorans. These sequences have GenBank Nos. ABD26841.1 (SEQ ID NO:101); ABD26530.1 (SEQ ID NO:539); ABD27301.1 (SEQ ID NO:541); and ABD27309.1 (SEQ ID NO:545).

One of skill will appreciate that the bioinformatic screen that was used to help identify putative enzymes is not a definitive predictor in itself of biochemical activities, particularly in view of (i) having only one known active enzyme for LigE in a different species, (ii) one known active enzyme for LigF, and (iii) the unexpected extent of such activities discovered. The tests for function therefore had to be performed empirically on the N. aromaticivorans putative beta-etherase gene set.

One of skill will also appreciate that the discovery of beta-etherase activities for all 4 N. aromaticivorans polypeptides was a complete surprise given the relatively low levels of identities (37%-62%) the sequences had with respect to the S. paucimobilis LigE and LigF proteins.

One of skill will also appreciate that the discovery of 2 novel beta-etherases from the N. aromaticivorans with improved activities over the corresponding LigE and LigF proteins from S. paucimobilis were completely unexpected, and this exciting discovery provides a foundation for further enzyme development for industrial applications.

### Example 5

This example describes the extended use of bioinformatics to identify a pool of putative enzymes in the discovery program. As noted above, the bioinformatic screen that was used to help identify putative enzymes initially was not a definitive predictor in itself of biochemical activities, particularly in view of (i) having only one known active enzyme for LigE in a different species, (ii) one known active enzyme for LigF, and (iii) the unexpected extent of such activities discovered. Having the additional known active enzymes provided more information that could be used to enhance the effectiveness of the bioinformatics in identifying the pool of putative enzymes for both LigE-type and LigF-type enzymes.

Sequence to function correlations for the newly discovered beta-etherases were analyzed and identified. A bioinformatic survey of functional domains, essential catalytic residues, and sequence alignments was performed for the N. aromaticivorans LigE and LigF polypeptides. While not intending to be bound by any theory or mechanism of action, the rationale and key results of the survey include at least the following:

Identifying Functional Domains

As shown in FIG. 4, high levels of beta-etherase activities were discovered for the N. aromaticivorans LigE1 and LigF2 polypeptide sequences compared to the S. paucimobilis LigE and LigF proteins. The N. aromaticivorans LigE1 and LigF2 polypeptide sequences were used as query sequences for the identification of functional domains using the Conserved Domain Database (CDD) in GenBank.

The N. aromaticivorans LigE1 polypeptide is annotated as a glutathione S-transferase (GST)-like protein with similarity to the GST_C family, and the beta-etherase LigE subfamily. The LigE sub-family is composed of proteins similar to S. paucimobilis beta etherase, LigE, a GST-like protein that catalyzes the cleavage of the beta-aryl ether linkages present in low-moleculer weight lignins using reduced glutathione (GSH) as the hydrogen donor in the reaction. The GST fold contains an N-terminal thioredoxin-fold domain and a C-terminal alpha helical domain, with an active site located in a cleft between the two domains.

Table 5 describes conserved domains and essential amino acid residues in the N. aromaticivorans LigE1 polypeptide (ABD26841.1), according to some embodiments. The three (3) conserved functional domains annotated in the N. aromaticivorans LigE1 polypeptide are: i) the dimer interface; ii) the N terminal domain; iii) the lignin substrate binding pocket or the H site. Amino acid residues defining the functional domains in such embodiments are residues 98-221 in the N. aromaticivorans LigE1 polypeptide.

Table 5 also lists fifteen (15) amino acid residues as conserved and essential for catalytic activity (column 3 of Table 5), according to some embodiments. These include: K100; A101; N104; P166; W107; Y184; Y187; R188; G191; G192; F195; V111; G112; M115; F116. While not intending to be bound by any theory or mechanism of action, these residues appear responsible for the high beta-etherase catalytic activity discovered for the N. aromaticivorans LigE1 polypeptide compared to the S. paucimobilis ligE polypeptide.

In such embodiments, the essential amino acid residues of the N. aromaticivorans LigE1 polypeptide might be altered conservatively, and singly or in combination with similar amino acid residues that would retain or improve the catalytic function of the N. aromaticivorans LigE1 polypeptide. Examples of such alternate residues that might be incorporated at the essential positions are also shown in column 4 of Table 5.

TABLE 5

| | | Conserved | |
|---|---|---|---|
| | Residues | residues | Alternate |
| | defining the | essential for | residues |
| | domain in N. | catalysis in N. | suggested for |
| Functional | aromaticivorans | aromaticivorans | the essential |
| domain | LigE1 | LigE1 | positions |
| Dimer | (residues 98-221 | K100; A101; | K100->R |
| interface | of SEQ ID | N104; P166 | A101->L; I; V; G; S |
| | NO: 101) | | N104->Q; H; S; A |
| N terminal | (residues 98-221 | K100; W107; | K100->R |
| domain | of SEQ ID | Y184; Y187; | W107->Y; F; A; S |
| interface | NO: 101) | R188; G191; | Y184->W; F; A; S |
| | | F195 | Y187-> W; F; A; S |
| | | | R188->K |
| | | | G191-> L; I; V; A; S |
| | | | F195->W; Y; A; S |
| Lignin/ | (residues 98-221 | W107; V111; | W107->Y; F; A; S |
| substrate | of SEQ ID | G112; M115; | V111-> L; I; G; A; S |
| binding | NO: 101) | F116; G192; | G112-> L; I; V; A; S |
| pocket or | | F195 | M115->S; A; G |
| H site | | | G192-> L; I; V; A; S |
| | | | F195-> W; Y; A; S |

The N. aromaticivorans LigF2 polypeptide is annotated as a glutathione S-transferase (GST)-like protein with similarity to the GST_C family, catalyzing the conjugation of glutathione with a wide range of xenobiotic agents.

Table 6 describes conserved domains and essential amino acid residues in the N. aromaticivorans LigF2 polypeptide (ABD27301.1), according to some embodiments. The three (3) conserved functional domains annotated for the N. aromaticivorans LigF2 polypeptide are similar to those described for the N. aromaticivorans LigE polypeptide and comprise: i) the dimer interface; ii) the N terminal domain; iii) the substrate binding pocket or the H site. In such embodiments, amino acid residues defining the functional domains are residues 99-230 in the N. aromaticivorans LigF2 polypeptide.

Table 6 also lists sixteen (16) amino acid residues as conserved and essential for catalytic activity (column 3 of Table 6) of the N. aromaticivorans LigF2 polypeptide, according to some embodiments. These include: R100; Y101; K104; K176; D107; L194; I197; N198; S201; M206; M111; N112; S115; M116; M206; H202. While not intending to be bound by any theory or mechanism of action, these 16 residues appear to be responsible for the high beta-etherase catalytic activity discovered for the N. aromaticivorans LigF2 polypeptide compared to the S. paucimobilis LigF polypeptide.

In such embodiments, the essential amino acid residues of the N. aromaticivorans LigF2 polypeptide might be altered conservatively, and singly or in combination with similar amino acid residues that would retain or improve the catalytic function of the N. aromaticivorans LigF2 polypeptide. Examples of such alternate residues that might be incorporated at the essential positions are shown in column 4 of Table 6.

TABLE 6

| | Residues | Conserved | Alternate |
|---|---|---|---|
| | defining the | residues essential | residues |
| | domain | for catalysis | suggested |
| | in N. | in N. | for the |
| Functional | aromaticivorans | aromaticivorans | essential |
| domain | LigF2 | LigF2 | positions |
| Dimer | (residues 99-230 | R100; Y101; | R100->K |
| interface | of SEQ ID | K104; K176 | Y101-> W; F; A; S |
| | NO: 541) | | K104->R |
| | | | K176->R |
| N terminal | (residues 99-230 | R100; D107; | R100->K |
| domain | of SEQ ID | L194 ;I197; | D107->E |
| interface | NO: 541) | N198; S201; | L194-> V; I; G; A; S |
| | | M206 | I197-> L; V; G; A; S |
| | | | N198->Q |
| | | | S201->A; M; G |
| | | | M206->S; A; G |
| Substrate | (residues 99-230 | D107; M111; | D107->E |
| binding | of SEQ ID | N112; S115; | M111->S; A; G |
| pocket | NO: 541) | M116; M206; | N112->Q |
| or H site | | H202 | S115->A; M; G |
| | | | M116->S; A; G |
| | | | M206->S; A; G |
| | | | H202->N; Q; S; M |

Identifying Additional Functional Domains

Bioinformatic methods were used to further understand the protein structure that may result in the desired activities. First, the LigE1 and LigF2 were analyzed together. Amino acid sequence alignments were performed using the N. aromaticivorans ligE1 (ABD26841.1) and ligF2 (ABD27301.1) sequences using the BLAST-P program in GenBank, and the ProDom and PraLine programs. Full length sequence alignments yielded hits with relatively low identities, for example, identities of <70%.

Next, regions in LigE1 and LigF2 were analyzed independently in GENBANK.

For LigE1, an alignment was performed against the database in GENBANK using the following query sequence: “tispfvwatkyalkhkgfdldvvpggftgilertgg” (residues 19-54 of SEQ ID NO:101), from N. aromaticivorans ligE1. The BLAST yielded at least 3 subject sequences with high identities in the thioredoxin (TRX)-like superfamily of proteins containing a TRX fold. Many members contain a classic TRX domain with a redox active CXXC motif.

Without intending to be bound by any theory or mechanism of action, they are thought to function as protein disulfide oxidoreductases (PDOs), altering the redox state of target proteins via the reversible oxidation of their active site dithiol. The PDO members of this superfamily include the families of TRX, protein disulfide isomerase (PDI), tIpA, glutaredoxin, NrdH redoxin, and bacterial Dsb proteins (DsbA, DsbC, DsbG, DsbE, DsbDgamma). Members of the superfamily that do not function as PDOs but contain a TRX-fold domain include phosducins, peroxiredoxins, glutathione (GSH) peroxidases, SCO proteins, GSH transferases (GST, N-terminal domain), arsenic reductases, TRX-like ferredoxins and calsequestrin, among others.

Table 7 lists 3 subject sequences having high identities (>80%) to residues 19-54 of LigE-1 (SEQ ID NO:101). In some embodiments, these sequences are likely to be essential to catalytic functions similar to those discovered for the N. aromaticivorans ligE1 polypeptide.

TABLE 7

| | | | Identity/Similarity to |
|---|---|---|---|
| | | | N. aromaticovorans |
| | | | LigE1 query |
| | | GenBank | sequence residues |
| Subject sequence | Species; Gene | accession # | 19-54 (%) |
| (residues 19-54 of SEQ ID | Sphingomonas | BAA02032.1 | 89/97 |
| NO: 1) | paucimobilis; beta | | |
| TISPYVWRTKYALKHKGFDI | etherase | | |
| DIVPGGFTGILERTGG | | | |
| (residues 19-54 of SEQ ID | Novosphingobium sp. | YP004533906.1 | 86/92 |
| NO: 89) | PP1Y; glutathione S | | |
| TISPFVWRTKYALAHKGFD | transferase like protein | | |
| VDIVPGGFTGIAERTGG | | | |
| (residues 19-54 of SEQ ID | Sphingobium sp. SYK- | BAJ11989.1 | 83/94 |
| NO: 3) | 6; | | |
| TISPFVWATKYAIAHKGFEL | beta-etherase | | |
| DIVPGGFSGIPERTGG | | | |

The nucleotide and amino acid sequences in Table 7 are incorporated herein by reference in their entirety through the GenBank Accession Numbers.

Likewise, for LigF2, separate alignments were performed against the database in GENBANK using the following 2 query sequences: “ainpegqvpvl” (residues 47-57 of SEQ ID NO:541); and “iithttvineyled” (residues 63-76 of SEQ ID NO:541), from N. aromaticivorans ligF2 (ABD27301.1) yielded multiple subject sequences with high identities in the GST-N superfamily of proteins. Without intending to be bound by any theory or mechanism of action, the N terminal region (residues 43-75 of SEQ ID NO:541) of the N. aromaticivorans ligF2 polypeptide is annotated in the CDD to encompass:

i. N terminal residues thought to make contact with the C terminal interface in forming the tertiary protein structure for the GST-N family of proteins;

ii. N terminal residues thought to be involved in dimerization of the polypeptides; and,

iii. Residues thought to be involved in the binding of glutathione substrate.

Table 8 provides the percent identities and similarities to N. aromaticovorans LigF2 query sequence residues 47-57.

TABLE 8

| | | | Identity/Similarity to |
|---|---|---|---|
| | | | N. aromaticovorans |
| | | | LigF2 |
| | | GenBank | query sequence |
| Subject sequence | Species; Gene | accession # | residues 47-57 (%) |
| (residues 45-55 of | Proteus mirabilis ATCC | ZP_03840063.1 | 91/91 |
| SEQ ID NO: 983) | 29906; glutathione S- | | |
| AINPKGQVPVL | transferase | | |
| (residues 60-70 of | Neisseria macacae ATCC | ZP_08683997.1 | 82/91 |
| SEQ ID NO: 985) | 33926; glutathione S- | | |
| AINPQGQVPAL | transferase | | |
| (residues 43-53 of | Rhodospirillum rubrum; | YP_425114.1 | 82/91 |
| SEQ ID NO: 987) | glutathione S-transferase- | | |
| AMNPEGEVPVL | like protein | | |
| (residues 46-56 of | Neisseria sicca ATCC | ZP_05317369.1 | 82/91 |
| SEQ ID NO: 989) | 29256; glutathione S- | | |
| AINPQGQVPAL | transferase | | |
| (residues 46-56 of | Neisseria mucosa ATCC | ZP_05978410.1 | 82/91 |
| SEQ ID NO: 991) | 25996; glutathione S- | | |
| AINPQGQVPAL | transferase | | |
| (residues 19-29 of | alpha proteobacterium | ZP_02189431.1 | 82/91 |
| SEQ ID NO: 993) | BAL199; Glutathione S- | | |
| AINPAGEVPVL | transferase-like protein | | |
| (residues 31-41 of | Marinomonas sp. MED121; | ZP_01077889.1 | 91/91 |
| SEQ ID NO: 995) | glutathione S-transferase | | |
| AINPLGQVPVL | | | |
| (residues 46-55 of | Proteus penneri ATCC | ZP_03805830.1 | 90/90 |
| SEQ ID NO: 997) | 35198; hypothetical protein | | |
| INPKGQVPVL | PROPEN_04226 | | |
| (residues 45-55 of | AURANDRAFT_7474 | EGB13094.1 | 82/91 |
| SEQ ID NO: 999) | Aureococcus | | |
| AINPQGKVPVL | anophagefferens; | | |
| | hypothetical protein | | |

The nucleotide and amino acid sequences in Table 8 are incorporated herein by reference in their entirety through the GenBank Accession Numbers.

Table 9 provides the percent identities and similarities to N. aromaticovorans LigF2 query sequence residues 63-76.

TABLE 9

| | | | Identity/Similarity to |
|---|---|---|---|
| | | | N. aromaticovorans |
| | | GenBank | LigF2 query sequence |
| Subject sequence | Species; Gene | accession # | residues 63-76 (%) |
| (residues 107-115 of | Trichophyton verrucosum | XP_003019921.1 | 100/100 |
| SEQ ID NO: 1001) | HKI 0517; conserved | | |
| TVINEYLED | hypothetical protein | | |
| (residues 103-111 of | Arthroderma benhamiae | XP_003017304.1 | 100/100 |
| SEQ ID NO: 1003) | CBS 112371; conserved | | |
| TVINEYLED | hypothetical protein | | |
| (residues 72-80 of | Trichophyton rubrum CBS | XP_003232549.1 | 100/100 |
| SEQ ID NO: 1005) | 118892; glutathione | | |
| TVINEYLED | transferase | | |
| (residues 62-75 of | Novosphingobium sp. PP1Y; | YP_004533905.1 | 79/79 |
| SEQ ID NO: 1007) | glutathione S-transferase- | | |
| IITESTVICEYLED | like protein | | |
| (residues 84-92 of | Arthroderma gypseum CBS | XP_003171868.1 | 89/100 |
| SEQ ID NO: 1009) | 118893; hypothetical protein | | |
| TVINEFLED | MGYG_06412 | | |
| (residues 61-69 of | Trichophyton equinum CBS | EGE04518.1 | 89/100 |
| SEQ ID NO: 1011) | 127.97; hypothetical protein | | |
| TVINEFLED | TEQG_03389 | | |

The nucleotide and amino acid sequences in Table 9 are incorporated herein by reference in their entirety through the GenBank Accession Numbers.

The bioinformatics provides valuable information about protein structure that can assist in identifying test candidates. For example, the LigE1 has the 98-221 region, which is annotated in the databases as potentially responsible as component of binding and activity, dimerization, and for binding and catalysis in general. While not intending to be bound by any theory or mechanism of action, the variability in active site structures is reflected by the variability in substrate structures. Likewise, upon further research using bioinformatics, it was further discovered that the 19-54 region, which is annotated in the databases as a second region that is potentially responsible as component of the reductase function, and thus potentially responsible for catalysis in addition to the 98-221 region, while having more conservation between members.

Obtaining additional structural information that will assist in finding high performing proteins within each family of strains is within the scope of the teachings to the extent that the methodology is known to one of skill. A variety of research techniques are known to one of skill. Bioinformatic methods, such as motif finding, are an example of one way to obtain the additional structural information. Motif finding, also known as profile analysis, constructs global multiple sequence alignments that attempt to align short conserved sequence motifs among the sequences in the query set. This can be done, for example, by first constructing a general global multiple sequence alignment, after which highly conserved regions are isolated, in a manner similar to what is taught herein, and used to construct a set of profile matrices. The profile matrix for each conserved region is arranged like a scoring matrix but its frequency counts for each amino acid or nucleotide at each position are derived from the conserved region's character distribution rather than from a more general empirical distribution. The profile matrices are then used to search other sequences for occurrences of the motif they characterize.

LigE-1 and LigF-2 were further examined by comparing their structures to other polypeptides of the LigE-type and LigF-type, respectively. Table 10A shows conserved residues between the polypeptide sequences of LigE and LigE-1, and Table 10B shows shows conserved residues between the polypeptide sequences of LigF and LigF-2.

TABLE 10A

| Res | Pos |
|---|---|
| M | 1 |
| A | 2 |
| N | 4 |
| N | 5 |
| T | 6 |
| I | 7 |
| T | 8 |
| Y | 10 |
| D | 11 |
| L | 12 |
| L | 14 |
| G | 17 |
| T | 19 |
| I | 20 |
| S | 21 |
| P | 22 |
| V | 24 |
| W | 25 |
| T | 27 |
| K | 28 |
| Y | 29 |
| A | 30 |
| L | 31 |
| K | 32 |
| H | 33 |
| K | 34 |
| G | 35 |
| F | 36 |
| D | 37 |
| D | 39 |
| V | 41 |
| P | 42 |
| G | 43 |
| G | 44 |
| F | 45 |
| T | 46 |
| G | 47 |
| I | 48 |
| L | 49 |
| E | 50 |
| R | 51 |
| T | 52 |
| G | 53 |
| G | 54 |
| E | 57 |
| R | 58 |
| P | 60 |
| I | 62 |
| V | 63 |
| D | 64 |
| D | 65 |
| G | 66 |
| E | 67 |
| V | 69 |
| L | 70 |
| D | 71 |
| S | 72 |
| W | 73 |
| I | 75 |
| E | 77 |
| Y | 78 |
| L | 79 |
| D | 80 |
| K | 82 |
| Y | 83 |
| P | 84 |
| D | 85 |
| R | 86 |
| P | 87 |
| L | 89 |
| K | 100 |
| L | 102 |
| D | 103 |
| N | 104 |
| W | 105 |
| W | 107 |
| A | 110 |
| V | 111 |
| G | 112 |
| P | 113 |
| W | 114 |
| C | 117 |
| D | 121 |
| Y | 122 |
| D | 124 |
| L | 125 |
| S | 126 |
| L | 127 |
| P | 128 |
| Q | 129 |
| D | 130 |
| Y | 133 |
| V | 134 |
| S | 137 |
| R | 138 |
| E | 139 |
| L | 148 |
| E | 149 |
| V | 151 |
| Q | 152 |
| A | 153 |
| G | 154 |
| R | 155 |
| E | 156 |
| R | 158 |
| L | 159 |
| P | 160 |
| L | 166 |
| E | 167 |
| P | 168 |
| R | 170 |
| L | 173 |
| A | 174 |
| W | 178 |
| L | 179 |
| G | 180 |
| G | 181 |
| P | 184 |
| N | 185 |
| A | 187 |
| D | 188 |
| Y | 189 |
| T | 198 |
| A | 199 |
| S | 200 |
| V | 201 |
| T | 204 |
| P | 205 |
| L | 207 |
| D | 210 |
| D | 211 |
| P | 212 |
| L | 213 |
| R | 214 |
| D | 215 |
| W | 216 |
| R | 219 |
| D | 222 |
| L | 223 |
| G | 226 |
| L | 227 |
| G | 228 |
| R | 229 |
| H | 230 |
| P | 231 |
| G | 232 |
| P | 235 |
| L | 236 |
| F | 237 |
| G | 238 |
| L | 239 |
| R | 242 |
| E | 243 |
| G | 244 |
| D | 245 |
| P | 246 |
| F | 249 |
| R | 251 |
| G | 254 |
| G | 257 |
| N | 264 |
| G | 266 |
| P | 267 |
| T | 270 |
| R | 275 |
| E | 278 |

As can be seen, there is a high degree of between-species similarity between LigE and LigE-1 in the LigE-type family. The LigE residues are from S. paucimobilis (BAA02032.1) and the LigE-1 residues are from N. aromaticivorans LigE1 (ABD26841.1). The numbering is done according to the S. paucimobilis sequence (BAA02032.1) in the PRALINE alignment file (gaps not included).

TABLE 10B

| Res | Pos |
|---|---|
| M | 1 |
| Y | 6 |
| P | 10 |
| A | 12 |
| N | 13 |
| S | 14 |
| K | 16 |
| L | 21 |
| E | 23 |
| K | 24 |
| G | 25 |
| L | 26 |
| E | 29 |
| D | 34 |
| F | 38 |
| E | 39 |
| H | 41 |
| F | 45 |
| I | 48 |
| N | 49 |
| P | 50 |
| G | 52 |
| V | 54 |
| P | 55 |
| T | 65 |
| T | 68 |
| I | 70 |
| E | 72 |
| Y | 73 |
| L | 74 |
| E | 75 |
| D | 76 |
| L | 85 |
| P | 87 |
| D | 89 |
| R | 97 |
| W | 99 |
| K | 101 |
| L | 161 |
| K | 167 |
| E | 176 |
| L | 179 |
| L | 185 |
| Y | 190 |
| L | 192 |
| A | 193 |
| D | 194 |
| I | 195 |
| P | 221 |
| L | 223 |
| W | 226 |
| R | 229 |
| R | 233 |
| P | 234 |
| A | 235 |

As can be seen, there is less between-species similarity between LigF and LigF-2 in the LigF-type family. The LigF residues are from S. paucimobilis (BAA02031.1) and the LigF-2 residues are from N. aromaticivorans (ABD27301.1). Numbering is according to the S. paucimobilis sequence (BAA02031.1) in the PRALINE alignment file (gaps not included.

### Example 6

This example provides additional sequences for a second round of assays, the sequences containing the 3 conserved functional domains described herein for the GST_C family of proteins, and belong to the beta-etherase LigE subfamily. Table 11 lists nine (9) additional sequences having identities of 51%-73% at the amino acid level that were identified in the SwissProt database using the S. paucimobilis LigE sequence (P27457.3) as the query. The bioinformatics information suggests that these 9 sequences are excellent candidates for the next round of synthesis, cloning, expression and testing for the desired biochemical functions using the methods described herein.

TABLE 11

| | | | Identity to |
|---|---|---|---|
| | | | S. paucimobilis |
| | | | LigE |
| | | Accession # | polypeptide |
| | Annotation | SwissProt/GenBank | (%) |
| 7 | Dianthus caryophyllus; | P28342.1/121736 | 59 |
| | Glutathione S transferase | | |
| 8 | Euforbua esula; | P57108.1/11132235 | 51 |
| | Glutathione S transferase | | |
| 9 | Zea mays; Glutathione | P04907.4/1170090 | 70 |
| | S transferase | | |
| 10 | Pseudomonas aeruginosa; | P57109.1/11133449 | 58 |
| | Maleylacetoacetate | | |
| | isomerase | | |
| 11 | Zea mays; Glutathione | P46420.2/1170092 | 63 |
| | S transferase | | |
| 12 | Arabidopsis thaliana; | Q8L7C9.1/75329755 | 61 |
| | Glutathione S transferase | | |
| 13 | Arabidopsis thaliana; | P42769.1/1170093 | 73 |
| | Glutathione S transferase | | |
| 14 | Oryza sativa Japonica | O65857.2/57012737 | 59 |
| | Group; Probable | | |
| | Glutathione S transferase | | |
| 15 | Oryza sativa Japonica | O82451.3/57012739 | 62 |
| | Group; Probable | | |
| | Glutathione S transferase | | |

The nucleotide and amino acid sequences in Table 11 are incorporated herein by reference in their entirety through the GenBank Accession Numbers.

### Example 7

This example describes how native lignin core structures can be hydrolyzed by the action of C alpha-dehydrogenases, beta-etherases, and glutathione-eliminating enzymes.

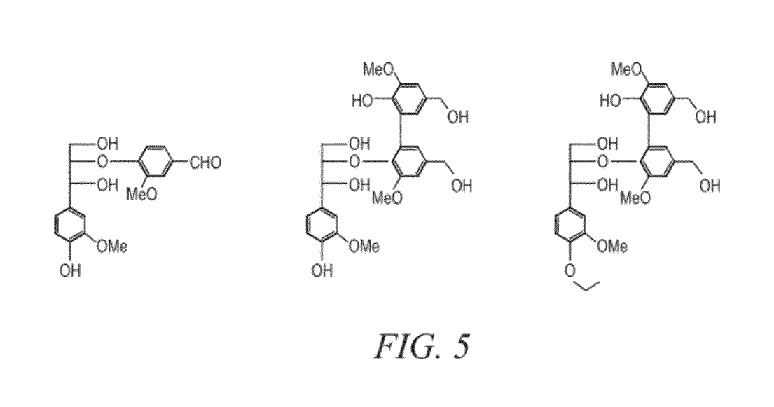

FIG. 5 illustrates beta-aryl-ether compounds to be tested as substrates representing native lignin structures, according to some embodiments. While MUAV was used as a model substrate in the identification of novel beta-etherase enzymes, additional aryl-ether compounds such as those shown in FIG. 5 might be used to assess substrate specificities of the beta-etherases towards dimers and trimers of aromatic compounds containing the beta-aryl ether linkage and representative of native lignin structures. Higher order oligomers of molecular weights <2000 might be synthesized and tested as well. The compounds might be obtained by custom organic synthesis, as for the fluorescent substrate MUAV.

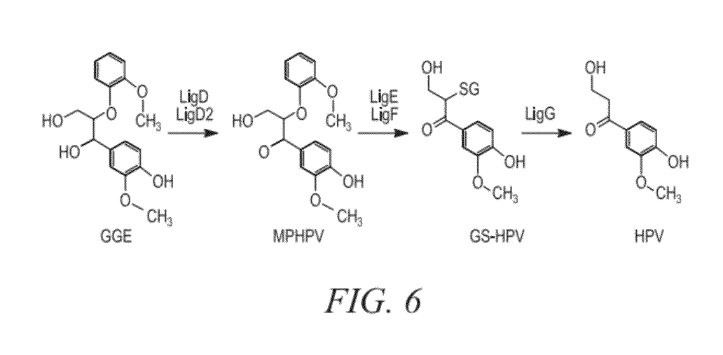

FIG. 6 illustrates pathways of guaiacylglycerol-β-guaiacyl ether (GGE) metabolism by S. paucimobilis, according to some embodiments. Enzymes in addition to LigE/F-like beta etherases might be required to hydrolyze native lignin core structures. The model β-aryl ether compound guaiacylglycerol-β-guaiacyl ether (GGE) is believed to contain the main chemical linkages present in native lignin, including the hydroxyl, aryl-ether and methoxy functionalities. The biotransformation of GGE to the lignin monomer beta-hydroxypropiovanillone (beta-HPV) is partially understood for S. paucimobilis, and proposed to occur via the action of 3 separate enzymes in a step-wise manner. The ligD gene product encodes a C alpha-dehydrogenase which oxidizes GGE to α-(2-methoxyphenoxy)-β-hydroxypropiovanillone (MPHPV); the ether bond of MPHPV is cleaved by the beta-etherase activities of the ligE and ligF gene products to yield the lignin monomer guaiacol, and α-glutathionylhydroxypropiovanillone (GS-HPV), respectively. The ligG gene product encodes a glutathione (GSH)-eliminating glutathione S transferase (GST) which catalyzes the elimination of glutathione (GSH) from GS-HPV to yield the lignin hydroxypropiovanillone (HPV).

While the LigE and LigF polypeptides, or similar ones described herein, might be sufficient to hydrolyze native lignin structures, it would be useful to discover novel C alpha dehydrogenases (S. paucimobilis LigD homologs) and glutathione (GSH)-eliminating glutathione S transferases (S. paucimobilis LigG homologs) for industrial applications. The enzyme discovery programs might be conducted by methods similar to those described herein. The detection of lignin substrates, intermediates, and products of biochemical reactions might be measured following filtration, and the extraction of substrates and products into ethyl acetate. Substrates and products might be separated using reverse phase HPLC conditions with a C18 column developed with a gradient solvent system of methanol and water, and detected at 230 nm or 254 nm.

Table 12 lists potential C alpha-dehydrogenase polypeptide sequences, the LigD-type, for use in conjunction with beta etherases including, but not limited to, LigE/F. The sequences were identified using bioinformatic methods, such as those taught herein. These C alpha-dehydrogenases are classified in the CDD as short-chain dehydrogenase/reductases (SDRs) and are a functionally diverse family of oxidoreductases that have a single domain with a structurally conserved Rossmann fold (alpha/beta folding pattern with a central beta-sheet), an NAD(P)(H)-binding region, and a structurally diverse C-terminal region. Classical SDRs are typically about 250 residues long, while extended SDRs are approximately 350 residues. Sequence identity between different SDR enzymes are typically in the 15-30% range, but the enzymes share the Rossmann fold NAD-binding motif and characteristic NAD-binding and catalytic sequence patterns.

Without intending to be bound by any theory or mechanism of action, these enzymes are thought to catalyze a wide range of activities including the metabolism of steroids, cofactors, carbohydrates, lipids, aromatic compounds, and amino acids, and act in redox sensing. Classical SDRs have an TGXXX[AG]XG cofactor binding motif and a YXXXK active site motif, with the Tyr residue of the active site motif serving as a critical catalytic residue (Tyr-151, human prostaglandin dehydrogenase (PGDH) numbering). In addition to the Tyr and Lys, there is often an upstream Ser (Ser-138, PGDH numbering) and/or an Asn (Asn-107, PGDH numbering) contributing to the active site; while substrate binding is in the C-terminal region, which determines specificity.

Without intending to be bound by any theory or mechanism of action, the standard reaction mechanism is thought to be a 4-pro-S hydride transfer and proton relay involving the conserved Tyr and Lys, a water molecule stabilized by Asn, and nicotinamide. Extended SDRs have additional elements in the C-terminal region, and typically have a TGXXGXXG cofactor binding motif. Complex (multidomain) SDRs such as ketoreductase domains of fatty acid synthase can have a GGXGXXG NAD(P)-binding motif and an altered active site motif (YXXXN). Fungal type ketoacyl reductases can have a TGXXXGX(1-2)G NAD(P)-binding motif. Some atypical SDRs are thought to have lost catalytic activity and/or have an unusual NAD(P)-binding motif and missing or unusual active site residues. Reactions catalyzed within the SDR family can include isomerization, decarboxylation, epimerization, C═N bond reduction, dehydratase activity, dehalogenation, Enoyl-CoA reduction, and carbonyl-alcohol oxidoreduction.

TABLE 12

| | | | Identity/Similarity to |
|---|---|---|---|
| | | GenBank Accession | S. paucimobilis LigD |
| | Species | Numbers | polypeptide (%) |
| 1 | N. aromaticivorans | YP495487.1 | 78/88 |
| 2 | N. aromaticivorans | YP496072.1 | 39/58 |
| 3 | N. aromaticivorans | YP496073.1 | 39/59 |
| 4 | N. aromaticivorans | YP495984.1 | 35/56 |
| 5 | N. aromaticivorans | YP497149.1 | 38/58 |

The nucleotide and amino acid sequences in Table 12 are incorporated herein by reference in their entirety through the GenBank Accession Numbers.

Table 13 lists potential LigG (glutathione-eliminating)-like enzyme sequences for use in conjunction with beta etherases including, but not limited to, LigE/F. The sequences were identified using bioinformatic methods, such as those taught herein. These might be utilized in conjunction with C-alpha dehydrogenases, and/or with LigE/F-like beta-etherases. The LigG-like proteins are annotated in the CDD as glutathione S-transferase (GST)-like proteins with similarity to the GST_C family, the GST-N family, and the thioredoxin (TRX)-like superfamily of proteins containing a TRX fold.

TABLE 13

| | | | Identity/Similarity to |
|---|---|---|---|
| | | GenBank Accession | S. paucimobilis LigG |
| | Species | Numbers | polypeptide (%) |
| 1 | N. aromaticovorans | YP_498160.1 | 23/41 |
| 2 | A. vinelandii DJ | YP_002798340 | 32/50 |

The nucleotide and amino acid sequences in Table 13 are incorporated herein by reference in their entirety through the GenBank Accession Numbers.

### Example 8

This example describes the creation of a novel recombinant microbial system for the conversion of lignin oligomers to monomers. Azotobacter vinelandii strain BAA-1303 DJ, for example, might be transformed with beta-etherase encoding genes from N. aromaticovorans with the objective of creating a lignin phenolics-tolerant A. vinelandii strain capable of converting lignin oligomers to monomers at high yields in industrial processes. Table 14 lists additional A. vinelandii strains that might be used as host strains for beta-etherase gene expression, for example, by their strain designation and American Type Culture Collection (ATCC) number.

TABLE 14

| | Strain | | | Strain | | | Strain | |
|---|---|---|---|---|---|---|---|---|
| | Desig- | ATCC | | Desig- | ATCC | | Desig- | ATCC |
| # | nation | Number | # | nation | Number | # | nation | Number |
| 1 | Wiscon- | 12518 | 8 | Ad116 | 17962 | 14 | B-6 | 7489 |
| | sin O | | | | | | | |
| 2 | 3a | 12837 | 9 | NRS 16 | 25308 | 15 | B-9 | 7492 |
| 3 | AV-3 | 13266 | 10 | UWD | 478 | 16 | 37 | 9046 |
| 4 | AV-4 | 13267 | 11 | 113 | 53800 | 17 | V1 | 7496 |
| 5 | AV-5 | 13268 | 12 | B-1 | 7484 | 18 | 3 | 9047 |
| 6 | OP | 13705 | 13 | B-4 | 7487 | | | |
| 7 | 135 | 53799 | — | — | — | — | — | — |
| | [VKMB- | | | | | | | |
| | 547] | | | | | | | |

The heterologous production of beta etherases, Cα dehydrogenases, and other enzymes for the production of lignin monomers and aromatic products in A. vinelandii might be achieved using the expression plasmid system described herein. The broad host range multicopy plasmid pKT230 (ATCC) encoding streptomycin resistance might be used for gene cloning. Genes can be synthesized by methods describe above, and cloned into the SmaI site of pKT230. The nifH promoter from A. vinelandii strain BAA 1303 DJ can be used to control gene expression.

A. vinelandii strain BAA 1303 DJ might be transformed with pKT230 derivatives using electroporation of electrocompetent cell (Eppendorf method), or by incubation of plasmid DNA with chemically competent cells prepared in TF medium (1.9718 g of MgSO4, 0.0136 g of CaSO4, 1.1 g of CH3COONH4, 10 g of glucose, 0.25 g of KH2PO4, and 0.55 g of K2HPO4 per liter). Transformants might be selected by screening for resistance to streptomycin. Gene expression might be induced by cell growth under nitrogen-free Burk's medium (0.2 g of MgSO4, 0.1 g of CaSO4, 0.5 g of yeast extract, 20 g of sucrose, 0.8 g of K2HPO4, and 0.2 g of KH2PO4, with trace amounts of FeCl3 and Na2MoO4, per liter).

The biochemical activity of a newly-discovered beta-etherase enzyme functionally expressed in A. vinelandii strain BAA 1303 DJ can be tested using methods known to one of skill, such as the methods provided herein. Biochemical activity assays for beta-etherase function, and for total protein might be performed as described herein.

### Example 9

This example describes the design and use of recombinant Azotobacter strains heterologously expressing enzymes for the production of high value aromatic compounds from lignin core structures. Table 15 lists a few examples of aromatic compounds that might be produced by the microbial platforms described herein.

TABLE 15

| | | Market | |
|---|---|---|---|
| Chemical | Market Volume | Value | |
| Product | (metric ton/year) | ($/lb) | Uses |
| 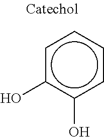 | 30 × 10³ | 2.34 | Antioxidant: 4- tert-butylcatechol. Flavors: piperonal; veratrol. Insecticides: carbofuran: propoxur. |
| 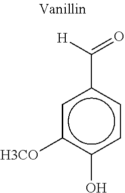 | 20 × 10³ | 6.12 | Flavor agent. Precursor for pharmaceutical methyldopa. |
| 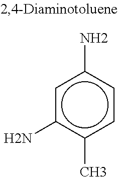 | 3 × 10⁶ | 1.65 | Precursor to toluene diisocyanates for urethane polymers. |
| 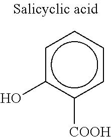 | 1.6 × 10³ (US) | 3.92 | Precursor to analgesic drug acetylsalicylic acid. Precursor to fragrances: amyl and methyl esters of salicylic acid. |
| 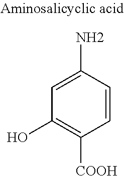 | | 57.38 | Tuberculosis drug. |
| 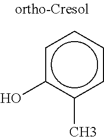 | 38 × 10³ | 0.8 | Precursors to herbicides: 4-chloro-2-methyl- phenoxyacetic acid; 2-(4-chloro-2- methylphenoxy)- propionic acid. |

One example of a microbial process to a commercial aromatic compound might be the production of catechol from lignin-derived phenolic compounds. Catechol might be produced from guaiacol using an A. vinelandii or A. chroococcum strain engineered with enzymes including beta-etherases and demethylases, or demethylase enzymes alone. Azotobacter strains might be engineered to express the heterologous enzymes by the methods described herein.

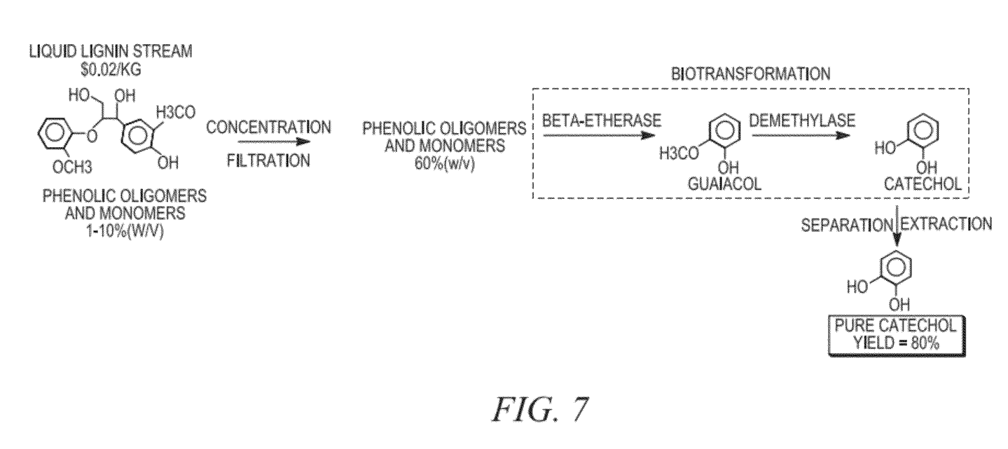

FIG. 7 illustrates an example of a biochemical process for the production of catechol from lignin oligomers, according to some embodiments. The biochemical processes leading to aromatic products such as catechol might be designed as 3 unit operations described below:

i) Fractionation of soluble lignin—Concentration or partial purification of soluble biorefinery lignin fractions or phenolic streams using methods known to one of skill.

ii) Biotransformation—The biotransformation of the phenolic substrate stream might be carried out in a fed-batch bioprocess using Azotobacter strains engineered to specifically and optimally convert specific lignin-derived phenolic substrates to the final product, such as catechol. Corn steep liquor might be used the base medium used in the biotransformations. The phenolic stream might be introduced in fed-batch mode, at concentrations that will be tolerated by the strains.

iii) Product separation—The product, such as catechol, might be purified from the aqueous culture broths using standard chemical separation methods such as liquid-liquid extractions (LLE) with solvents of varying polarities applied in a sequential manner.

Additional examples of designed biochemical routes to aromatic products are described below:

i) lignin-derived syringic acid might be converted to gallic acid via a 2-step biochemical conversion using aryl aldehyde oxidases and demethylases.

ii) Lignin-derived vanillin might be converted to protocatechuic acid via a 2-step biochemical conversion using aryl aldehyde oxidases and demethylases.

iii) Lignin-derived vanillin might be converted to catechol via a 3-step biochemical conversion using aryl aldehyde oxidases, aromatic decarboxylases, and demethylases.

iv) Lignin-derived 2-methoxytoluene might be converted to the urethane precursor 2,4-diaminotoluene via a 4-step biochemical conversion using demethylases, ferulate-5-hydroxylases, 2,4-nitrophenol oxidoreductases, and 2,4-nitrobenzene reductases.

In each case, the specific enzymes might be engineered into A. vinelandii or A. chroococcum strains, for example, and the process might be performed using unit operations similar to those described herein for the biochemical production of catechol.

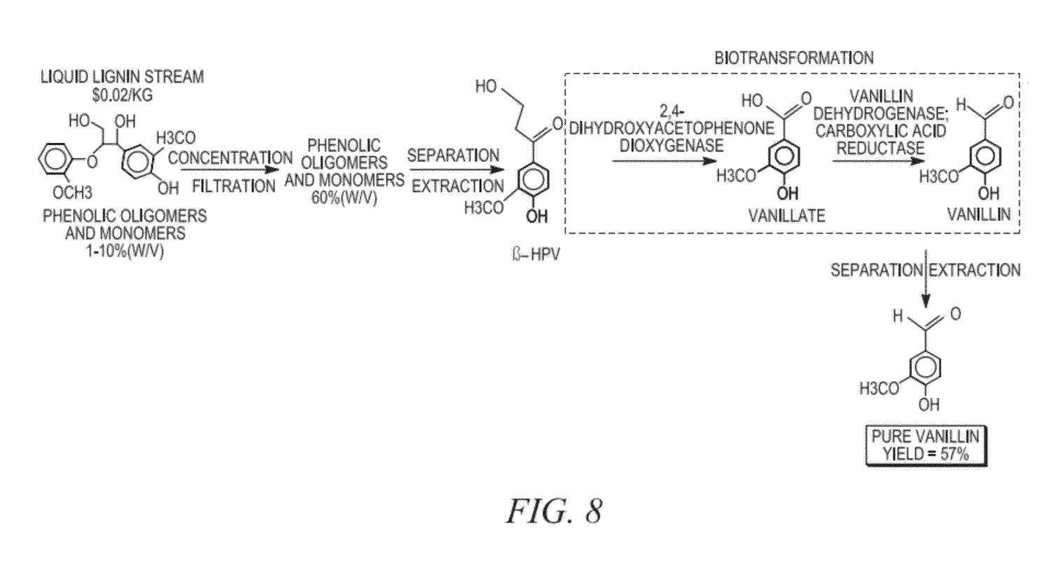

FIG. 8 illustrates an example of a biochemical process for the production of vanillin from lignin oligomers, according to some embodiments. Vanillin can be used as a flavoring agent, and as a precursor for pharmaceuticals such as methyldopa. Synthetic vanillin, for example, can be produced from petroleum-derived guaiacol by reaction with glyoxylic acid. Vanillin, however, can also be produced from lignin-derived β-hydroxypropiovanillone (β-HPV) according to the process scheme indicated in FIG. 8. A 2-step biochemical route to vanillin from β-HPV can be achieved using the enzymes 2,4-dihydroxyacetophenone oxidoreductase, and vanillin dehydrogenase or carboxylic acid reductases, engineered into A. vinelandii.

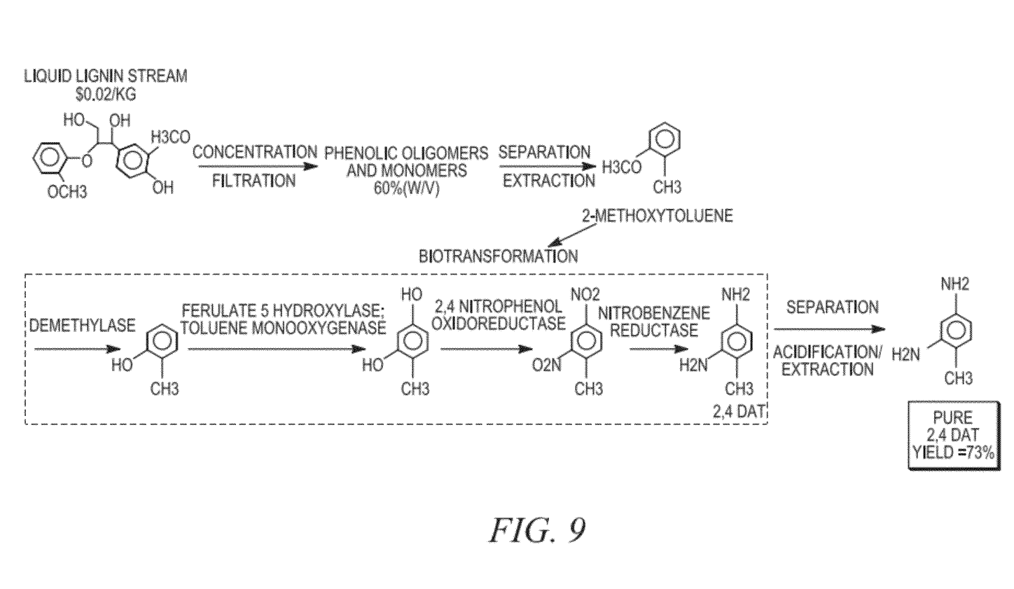

FIG. 9 illustrates an example of a biochemical process for the production of 2,4-diaminotoluene from lignin oligomers, according to some embodiments. Toluene diisocyanate (TDI) can be used in the manufacture of polyurethanes. For example, 2,4-diaminotoluene (2,4-DAT) is the key precursor to TDI. Diaminotoluenes can be produced industrially by the sequential nitration of toluene with nitric acid, followed by the reduction of the dinitrotoluenes to the corresponding diaminotoluenes. Both nitration and reduction reactions yield mixtures of toluene isomers from which the 2,4-DAT isomer is purified by distillation. The conversion of lignin-derived 2-methoxytoluene to 2,4-DAT can be achieved according to the process scheme outlined in FIG. 9. 2-methoxytoluene can be be converted to 2,4-DAT by A. vinelandii engineered with 4 enzymes to specifically demethylate, hydroxylate, nitrate and aminate methoxytoluene.

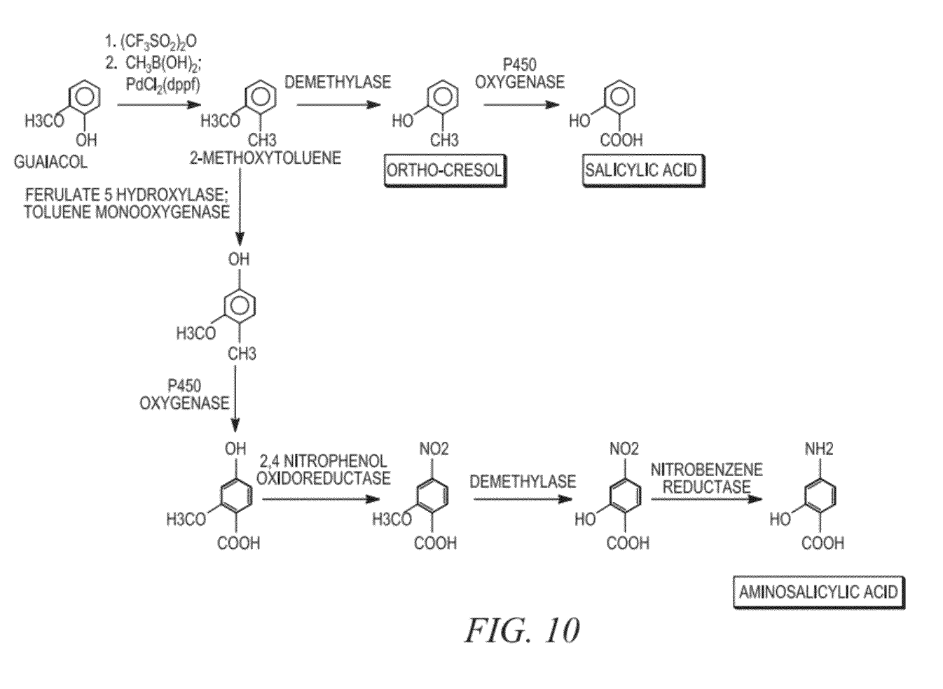

FIG. 10 illustrates process schemes for additional product targets that include ortho-cresol, salicylic acid, and aminosalicylic acid, for the production of valuable chemicals from lignin oligomers, according to some embodiments. These chemicals, as with the others, have traditionally been obtained from the problematic petrochemical processes. A few of the process schemes for producing these chemicals using the teachings herein, based on guaiacol or 2-methoxytoluene, are shown schematically in FIG. 10. Designed biochemical routes, combined with the remarkable phenolics-tolerance traits of Azotobacter strains are proposed for conversions of lignin structures to industrial and fine chemicals.

### Example 10

This example describes potential LigE-, LigF-, LigG-, and LigD-type polypeptides, and the genes encoding them. The potential polypeptides were identified using bioinformatic methods, such as those taught herein.

As described above, the query sequences in the initial pass for the LigE-type and LigF-type were Sphingomonas paucimobilis sequences, such as those discussed in Masai, E., et al. Likewise, the query sequences for the LigG-type and LigD-type were also Sphingomonas paucimobilis sequences, such as those discussed in Masai. The following sequences were used in the initial pass for all queries:

LigE, from Accession No BAA2032.1, is listed herein as SEQ ID NO:1 for the protein and SEQ ID NO:2 for the gene.

LigF, from Accession No BAA2031.1 (P30347.1), is listed herein as SEQ ID NO:513 for the protein and SEQ ID NO:514 for the gene.

LigG, from Accession No Q9Z339.2, is listed herein as SEQ ID NO:733 for the protein and SEQ ID NO:734 for the gene.

LigD, from Accession No Q01198.1, is listed herein as SEQ ID NO:777 for the protein and SEQ ID NO:778 for the gene.

The following sequences were used in a modified query to further refine the LigE-type and LigF-type, and the query sequences were the LigE-1 and LigF-2 that showed the surprising and unexpected results shown in FIG. 4: LigE-1, from Accession No ABD26841.1, is listed herein as SEQ ID NO:101 for the protein and SEQ ID NO:102 for the gene.

LigF-2, from Accession No ABD27301.1, is listed herein as SEQ ID NO:541 for the protein and SEQ ID NO:542 for the gene.

Table 16 lists SEQ ID NOs:1-246, which are potential protein sequences of the LigE-type, as well as a respective gene sequence encoding the protein. Table 17 lists SEQ ID NOs:247-576, which are potential protein sequences of the LigF-type, as well as a respective gene sequence encoding the protein. Table 18 lists SEQ ID NOs:577-776, which are potential protein sequences of the LigG-type, as well as a respective gene sequence encoding the protein. Table 19 lists SEQ ID NOs: 777-976, which are potential protein sequences of the LigD-type, as well as a respective gene sequence encoding the protein.

Bioinformatic methods, such as those described herein, can be used to suggest an efficient order of experimentation to identify additional potential enzymes for use with the teachings provided herein. Moreover, mutations and amino acid substitutions can be used to test affects on enzyme activity to further understand the structure of the most active proteins with respect to the enzyme functions sought by teachings provided herein.

TABLE 16

| PROTEIN | GENE | GENBANK | | |
|---|---|---|---|---|
| SEQ ID | SEQ ID | ACCESSION | | |
| NO: | NO: | NO: | DESCRIPTION: | TYPE |
| 1 | 2 | BAA02032.1 | Sphingomonas paucimobilis | LIGE |
| 3 | 4 | BAJ11989.1 | beta-etherase [Sphingobium sp. SYK-6] | LIGE |
| 5 | 6 | EFV85608.1 | glutathione S-transferase domain-containing | LIGE |
| | | | protein [Achromobacter xylosoxidans C54] | |
| 7 | 8 | EFW42705.1 | predicted protein [Capsaspora owczarzaki ATCC | LIGE |
| 9 | 10 | EGE55257.1 | Glutathione S-transferase domain-containing | LIGE |
| | | | protein [Rhizobium etli CNPAF512] | |
| 11 | 12 | EGP48556.1 | glutathione S-transferase domain-containing | LIGE |
| | | | protein [Achromobacter xylosoxidans AXX-A] | |
| 13 | 14 | EGP57475.1 | lignin degradation protein [Agrobacterium | LIGE |
| 15 | 16 | EGU12703.1 | Glutathione S-transferase [Rhodotorula glutinis | LIGE |
| | | | ATCC 204091] | |
| 17 | 18 | EGU56510.1 | glutathione S-transferase domain-containing | LIGE |
| | | | protein [Vibrio tubiashii ATCC 19109] | |
| 19 | 20 | NP_053324.1 | hypothetical protein pTi-SAKURA_p086 | LIGE |
| | | | [Agrobacterium tumefaciens] >dbj\|BAA87709.1\| | |
| | | | tiorf84 [Agrobacterium tumefaciens] | |
| 21 | 22 | NP_108131.1 | lignin beta-ether hydrolase [Mesorhizobium loti | LIGE |
| | | | MAFF303099] >dbj\|BAB54276.1\|lignin beta- | |
| | | | ether hydrolase [Mesorhizobium loti | |
| 23 | 24 | NP_354140.2 | lignin degradation protein [Agrobacterium | LIGE |
| | | | tumefaciens str. C58] >gb\|AAK86925.2\|lignin | |
| | | | degradation protein [Agrobacterium tumefaciens | |
| 25 | 26 | NP_385269.1 | putative BETA-etherase (BETA-aryl ether | LIGE |
| | | | cleaving enzyme) protein [Sinorhizobium meliloti | |
| | | | 1021] >emb\|CAC45742.1\|Putative beta- | |
| | | | etherase (beta-aryl ether cleaving enzyme) | |
| | | | protein [Sinorhizobium meliloti 1021] | |
| | | | >gb\|AEG03720.1\|Glutathione S-transferase | |
| | | | domain protein [Sinorhizobium meliloti BL225C] | |
| | | | >gb\|AEH79753.1\|putative BETA-etherase | |
| | | | [symbol] | |
| 27 | 28 | NP_774067.1 | ligninase [Bradyrhizobium japonicum USDA 110] | LIGE |
| | | | >dbj\|BAC52692.1\|ligE [Bradyrhizobium | |
| | | | japonicum USDA 110] | |
| 29 | 30 | NP_949676.1 | putative lignin beta-ether hydrolase | LIGE |
| | | | [Rhodopseudomonas palustris CGA009] | |
| | | | >emb\|CAE29781.1\|putative lignin beta-ether | |
| 31 | 32 | P27457.3 | RecName: Full = Beta-etherase; AltName: | LIGE |
| | | | Full = Beta-aryl ether cleaving enzyme | |
| | | | >gb\|AAA25878.1\|beta-etherase [Sphingomonas | |
| | | | paucimobilis] >dbj\|BAA02032.1\|beta-etherase | |
| 33 | 34 | [symbol] P_003028922. [symbol] | hypothetical protein SCHCODRAFT_85860 | LIGE |
| | | | [Schizophyllum commune H4-8] | |
| | | | >gb\|EFI94019.1\|hypothetical protein | |
| 35 | 36 | [symbol] P_003030384. [symbol] | hypothetical protein SCHCODRAFT_57691 | LIGE |
| | | | [Schizophyllum commune H4-8] | |
| | | | >gb\|EFI95481.1\|hypothetical protein | |
| 37 | 38 | [symbol] P_003033715. [symbol] | hypothetical protein SCHCODRAFT_81614 | LIGE |
| | | | [Schizophyllum commune H4-8] | |
| | | | >gb\|EFI98812.1\|hypothetical protein | |
| 39 | 40 | [symbol] P_003041213. [symbol] | hypothetical protein NECHADRAFT_55532 | LIGE |
| | | | [Nectria haematococca mpVI 77-13-4] | |
| | | | >gb\|EEU35500.1\|hypothetical protein | |
| | | | NECHADRAFT_55532 [Nectria haematococca | |
| 41 | 42 | XP_382462.1 | hypothetical protein FG02286.1 [Gibberella zeae | LIGE |
| 43 | 44 | [symbol] P_001207860. [symbol] | putative glutathione S-transferase (GST) | LIGE |
| | | | [Bradyrhizobium sp. ORS278] | |
| | | | >emb\|CAL79645.1\|putative glutathione S- | |
| 45 | 46 | [symbol] P_001236206. [symbol] | glutathione S-transferase domain-containing | LIGE |
| | | | protein [Acidiphilium cryptum JF-5] | |
| | | | >gb\|ABQ32287.1\|Glutathione S-transferase, N- | |
| | | | terminal domain protein [Acidiphilium cryptum JF [symbol] | |
| 47 | 48 | [symbol] P_001237901. [symbol] | putative glutathione S-transferase | LIGE |
| | | | [Bradyrhizobium sp. BTAi1] >gb\|ABQ33995.1\| | |
| | | | putative glutathione S-transferase (GST) | |
| 49 | 50 | [symbol] P_001262153. [symbol] | hypothetical protein Swit_1652 [Sphingomonas | LIGE |
| | | | wittichii RW1] >gb\|ABQ68015.1\|hypothetical | |
| | | | protein Swit_1652 [Sphingomonas wittichii RW1] | |
| 51 | 52 | [symbol] P_001326465. [symbol] | glutathione S-transferase domain-containing | LIGE |
| | | | protein [Sinorhizobium medicae WSM419] | |
| | | | >gb\|ABR59630.1\|Glutathione S-transferase | |
| | | | domain [Sinorhizobium medicae WSM419] | |
| 53 | 54 | [symbol] P_001413220. [symbol] | glutathione S-transferase domain-containing | LIGE |
| | | | protein [Parvibaculum lavamentivorans DS-1] | |
| | | | >gb\|ABS63563.1\|Glutathione S-transferase | |
| | | | domain [Parvibaculum lavamentivorans DS-1] | |
| 55 | 56 | [symbol] P_001526182. [symbol] | glutathione S-transferase [Azorhizobium | LIGE |
| | | | caulinodans ORS 571] >dbj\|BAF89264.1\| | |
| | | | glutathione S-transferase [Azorhizobium | |
| 57 | 58 | [symbol] P_001616516. [symbol] | lignin degradation protein [Sorangium cellulosum | LIGE |
| | | | ‘So ce 56’] >emb\|CAN96036.1\|lignin | |
| | | | degradation protein [Sorangium cellulosum ‘So | |
| 59 | 60 | [symbol] P_001772944. [symbol] | glutathione S-transferase domain-containing | LIGE |
| | | | protein [Methylobacterium sp. 4-46] | |
| | | | >gb\|ACA20510.1\|Glutathione S-transferase | |
| 61 | 62 | [symbol] P_001833458. [symbol] | glutathione S-transferase domain-containing | LIGE |
| | | | protein [Beijerinckia indica subsp. indica ATCC | |
| | | | 9039] >gb\|ACB95969.1\|Glutathione S- | |
| | | | transferase domain [Beijerinckia indica subsp. | |
| 63 | 64 | [symbol] P_001977695. [symbol] | beta-aryl ether cleaving enzyme, lignin | LIGE |
| | | | degradation protein [Rhizobium etli CIAT 652] | |
| | | | >gb\|ACE90517.1\|beta-aryl ether cleaving | |
| | | | enzyme, lignin degradation protein [Rhizobium | |
| 65 | 66 | [symbol] P_001993784. [symbol] | glutathione S-transferase domain-containing | LIGE |
| | | | protein [Rhodopseudomonas palustris TIE-1] | |
| | | | >gb\|ACF03309.1\|Glutathione S-transferase | |
| | | | domain [Rhodopseudomonas palustris TIE-1] | |
| 67 | 68 | [symbol] P_002280598. [symbol] | glutathione S-transferase domain [Rhizobium | LIGE |
| | | | leguminosarum bv. trifolii WSM2304] | |
| | | | >gb\|ACI54372.1\|Glutathione S-transferase | |
| | | | domain [Rhizobium leguminosarum bv. trifolii | |
| 69 | 70 | [symbol] P_002290149. [symbol] | glutathione S-transferase [Oligotropha | LIGE |
| | | | carboxidovorans OM5] >ref\|YP_004631892.1\| | |
| | | | beta etherase [Oligotropha carboxidovorans | |
| | | | OM5] >gb\|ACI94284.1\|glutathione S- | |
| | | | transferase [Oligotropha carboxidovorans OM5] | |
| | | | >gb\|AEI02075.1\|putative beta etherase | |
| | | | [Oligotropha carboxidovorans OM4] | |
| | | | [symbol] | |
| 71 | 72 | [symbol] P_002362903. [symbol] | glutathione S-transferase domain-containing | LIGE |
| | | | protein [Methylocella silvestris BL2] | |
| | | | >gb\|ACK51541.1\|glutathione S-transferase | |
| 73 | 74 | [symbol] P_002502105. [symbol] | glutathione S-transferase domain-containing | LIGE |
| | | | protein [Methylobacterium nodulans ORS 2060] | |
| | | | >gb\|ACL61802.1\|Glutathione S-transferase | |
| | | | domain protein [Methylobacterium nodulans | |
| 75 | 76 | [symbol] P_002549116. [symbol] | lignin degradation protein [Agrobacterium vitis | LIGE |
| | | | S4] >gb\|ACM36110.1\|lignin degradation protein | |
| | | | [Agrobacterium vitis S4] | |
| 77 | 78 | [symbol] P_002797805. [symbol] | glutathione S-transferase-like protein | LIGE |
| | | | [Azotobacter vinelandii DJ] >gb\|ACO76830.1\| | |
| | | | Glutathione S-transferase-like protein | |
| 79 | 80 | [symbol] P_002825455. [symbol] | putative lignin beta-ether hydrolase | LIGE |
| | | | [Sinorhizobium fredii NGR234] | |
| | | | >gb\|ACP24702.1\|putative lignin beta-ether | |
| 81 | 82 | [symbol] P_002975056. [symbol] | glutathione S-transferase domain protein | LIGE |
| | | | [Rhizobium leguminosarum bv. trifolii WSM1325] | |
| | | | >gb\|ACS55517.1\|Glutathione S-transferase | |
| | | | domain protein [Rhizobium leguminosarum bv. | |
| 83 | 84 | [symbol] P_004278359. [symbol] | lignin degradation protein [Agrobacterium sp. | LIGE |
| | | | H13-3] >gb\|ADY64039.1\|lignin degradation | |
| | | | protein [Agrobacterium sp. H13-3] | |
| 85 | 86 | [symbol] P_004285673. [symbol] | putative beta-etherase [Acidiphilium multivorum | LIGE |
| | | | AIU301] >dbj\|BAJ82791.1\|putative beta- | |
| | | | etherase [Acidiphilium multivorum AIU301] | |
| 87 | 88 | [symbol] P_004378290. [symbol] | glutathione S-transferase-like protein | LIGE |
| | | | [Pseudomonas mendocina NK-01] | |
| | | | >gb\|AEB56538.1\|glutathione S-transferase-like | |
| 89 | 90 | [symbol] P_004533906. [symbol] | glutathione S-transferase-like protein | LIGE |
| | | | [Novosphingobium sp. PP1Y] | |
| | | | >emb\|CCA92088.1\|glutathione S-transferase- | |
| 91 | 92 | [symbol] P_004548326. [symbol] | glutathione S-transferase domain-containing | LIGE |
| | | | protein [Sinorhizobium meliloti AK83] | |
| | | | >gb\|AEG52712.1\|Glutathione S-transferase | |
| 93 | 94 | [symbol] P_004613710. [symbol] | glutathione S-transferase domain-containing | LIGE |
| | | | protein [Mesorhizobium opportunistum | |
| | | | WSM2075] >gb\|AEH89616.1\|Glutathione S- | |
| | | | transferase domain protein [Mesorhizobium | |
| 95 | 96 | YP_269568.1 | putative lignin beta-etherase [Colwellia | LIGE |
| | | | psychrerythraea 34H] >gb\|AAZ24120.1\|putative | |
| | | | lignin beta-etherase [Colwellia psychrerythraea | |
| 97 | 98 | YP_469001.1 | beta-aryl ether cleaving enzyme, lignin | LIGE |
| | | | degradation protein [Rhizobium etli CFN 42] | |
| | | | >gb\|ABC90274.1\|beta-aryl ether cleaving | |
| | | | enzyme, lignin degradation protein [Rhizobium | |
| 99 | 100 | YP_487746.1 | glutathione S-transferase-like protein | LIGE |
| | | | [Rhodopseudomonas palustris HaA2] | |
| | | | >gb\|ABD08835.1\|Glutathione S-transferase-like | |
| 101 | 102 | YP_497675.1 | glutathione S-transferase-like protein | LIGE |
| | | | [Novosphingobium aromaticivorans DSM 12444] | |
| | | | >gb\|ABD26841.1\|glutathione S-transferase-like | |
| | | | protein [Novosphingobium aromaticivorans DSM | |
| 103 | 104 | YP_533979.1 | glutathione S-transferase-like protein | LIGE |
| | | | [Rhodopseudomonas palustris BisB18] | |
| | | | >gb\|ABD89660.1\|glutathione S-transferase-like | |
| 105 | 106 | YP_574731.1 | glutathione S-transferase-like protein | LIGE |
| | | | [Chromohalobacter salexigens DSM 3043] | |
| | | | >gb\|ABE60032.1\|glutathione S-transferase-like | |
| | | | protein [Chromohalobacter salexigens DSM | |
| 107 | 108 | YP_723508.1 | glutathione S-transferase-like protein | LIGE |
| | | | [Trichodesmium erythraeum IMS101] | |
| | | | >gb\|ABG53035.1\|glutathione S-transferase-like | |
| 109 | 110 | YP_767183.1 | etherase [Rhizobium leguminosarum bv. viciae | LIGE |
| | | | 3841] >emb\|CAK07074.1\|putative etherase | |
| | | | [Rhizobium leguminosarum bv. viciae 3841] | |
| 111 | 112 | YP_783091.1 | glutathione S-transferase [Rhodopseudomonas | LIGE |
| | | | palustris BisA53] >gb\|ABJ08111.1\|Glutathione | |
| | | | S-transferase [Rhodopseudomonas palustris | |
| 113 | 114 | YP_915395.1 | glutathione S-transferase domain-containing | LIGE |
| | | | protein [Paracoccus denitrificans PD1222] | |
| | | | >gb\|ABL69699.1\|Glutathione S-transferase, N- | |
| | | | terminal domain [Paracoccus denitrificans | |
| 115 | 116 | ZP_02146530. [symbol] | putative beta-etherase (beta-aryl ether cleaving | LIGE |
| | | | enzyme) protein [Phaeobacter gallaeciensis | |
| | | | BS107] >gb\|EDQ11875.1\|putative beta- | |
| | | | etherase (beta-aryl ether cleaving enzyme) | |
| 117 | 118 | ZP_02149699. [symbol] | putative beta-etherase (beta-aryl ether cleaving | LIGE |
| | | | enzyme) protein [Phaeobacter gallaeciensis | |
| | | | 2.10] >gb\|EDQ08644.1\|putative beta-etherase | |
| | | | (beta-aryl ether cleaving enzyme) protein | |
| 119 | 120 | ZP_02166231. [symbol] | putative beta-etherase (beta-aryl ether cleaving | LIGE |
| | | | enzyme) protein [Hoeflea phototrophica DFL-43] | |
| | | | >gb\|EDQ33834.1\|putative beta-etherase (beta- | |
| | | | aryl ether cleaving enzyme) protein [Hoeflea | |
| 121 | 122 | ZP_02190934. [symbol] | glutathione S-transferase-like protein [alpha | LIGE |
| | | | proteobacterium BAL199] >gb\|EDP62276.1\| | |
| | | | glutathione S-transferase-like protein [alpha | |
| 123 | 124 | ZP_03503368. [symbol] | Glutathione S-transferase domain [Rhizobium | LIGE |
| 125 | 126 | ZP_03507162. [symbol] | Glutathione S-transferase domain [Rhizobium | LIGE |
| 127 | 128 | ZP_03513891. [symbol] | Glutathione S-transferase domain [Rhizobium | LIGE |
| 129 | 130 | ZP_03519388. [symbol] | Glutathione S-transferase domain [Rhizobium | LIGE |
| 131 | 132 | ZP_03520502. [symbol] | putative etherase [Rhizobium etli GR56] | LIGE |
| 133 | 134 | ZP_05084767. [symbol] | glutathione S-transferase, N-terminal domain | LIGE |
| | | | [Pseudovibrio sp. JE062] >gb\|EEA94709.1\| | |
| | | | glutathione S-transferase, N-terminal domain | |
| 135 | 136 | ZP_06688745. [symbol] | lignin degradation protein [Achromobacter | LIGE |
| | | | piechaudii ATCC 43553] >gb\|EFF74366.1\|lignin | |
| | | | degradation protein [Achromobacter piechaudii | |
| 137 | 138 | ZP_06898146. [symbol] | glutathione S-transferase family protein | LIGE |
| | | | [Roseomonas cervicalis ATCC 49957] | |
| | | | >gb\|EFH10151.1\|glutathione S-transferase | |
| | | | family protein [Roseomonas cervicalis ATCC | |
| 139 | 140 | ZP_07027473. [symbol] | Glutathione S-transferase domain protein [Afipia | LIGE |
| | | | sp. 1NLS2] >gb\|EFI51229.1\|Glutathione S- | |
| | | | transferase domain protein [Afipia sp. 1NLS2] | |
| 141 | 142 | ZP_07373940. [symbol] | beta-etherase [Ahrensia sp. R2A130] | LIGE |
| | | | >gb\|EFL90585.1\|beta-etherase [Ahrensia sp. | |
| 143 | 144 | ZP_08328512. [symbol] | Glutathione S-transferase [gamma | LIGE |
| | | | proteobacterium IMCC1989] >gb\|EGG95341.1\| | |
| | | | Glutathione S-transferase [gamma | |
| 145 | 146 | ZP_08529965. [symbol] | lignin degradation protein [Agrobacterium sp. | LIGE |
| | | | ATCC 31749] >gb\|EGL63395.1\|lignin | |
| | | | degradation protein [Agrobacterium sp. ATCC | |
| 147 | 148 | ZP_08627134. [symbol] | lignin beta-ether hydrolase [Bradyrhizobiaceae | LIGE |
| | | | bacterium SG-6C] >gb\|EGP10168.1\|lignin beta- | |
| | | | ether hydrolase [Bradyrhizobiaceae bacterium | |
| 149 | 150 | ZP_08631370. [symbol] | Glutathione S-transferase domain-containing | LIGE |
| | | | protein [Acidiphilium sp. PM] >gb\|EGO96849.1\| | |
| | | | Glutathione S-transferase domain-containing | |
| 151 | 152 | ZP_08634908. [symbol] | Glutathione S-transferase domain-containing | LIGE |
| | | | protein [Acidiphilium sp. PM] >gb\|EGO93307.1\| | |
| | | | Glutathione S-transferase domain-containing | |
| 153 | 154 | ZP_08635074. [symbol] | glutathione S-transferase domain-containing | LIGE |
| | | | protein [Halomonas sp. TD01] >gb\|EGP21558.1\| | |
| | | | glutathione S-transferase domain-containing | |
| 155 | 156 | EGN93792.1 | hypothetical protein SERLA73DRAFT_115219 | LIGE |
| | | | [Serpula lacrymans var. lacrymans S7.3] | |
| | | | >gb\|EGO19163.1\|hypothetical protein | |
| | | | SERLADRAFT_453680 [Serpula lacrymans var. | |
| 157 | 158 | EGN94392.1 | hypothetical protein SERLA73DRAFT_188253 | LIGE |
| | | | [Serpula lacrymans var. lacrymans S7.3] | |
| | | | >gb\|EGO19875.1\|hypothetical protein | |
| | | | SERLADRAFT_478300 [Serpula lacrymans var. | |
| 159 | 160 | EGN96317.1 | hypothetical protein SERLA73DRAFT_186005 | LIGE |
| | | | [Serpula lacrymans var. lacrymans S7.3] | |
| | | | >gb\|EGO21854.1\|hypothetical protein | |
| | | | SERLADRAFT_474829 [Serpula lacrymans var. | |
| 161 | 162 | EGN96924.1 | hypothetical protein SERLA73DRAFT_185168 | LIGE |
| | | | [Serpula lacrymans var. lacrymans S7.3] | |
| | | | >gb\|EGO22516.1\|hypothetical protein | |
| | | | SERLADRAFT_473468 [Serpula lacrymans var. | |
| 163 | 164 | EGO00367.1 | hypothetical protein SERLA73DRAFT_107446 | LIGE |
| | | | [Serpula lacrymans var. lacrymans S7.3] | |
| | | | >gb\|EGO25928.1\|hypothetical protein | |
| | | | SERLADRAFT_415302 [Serpula lacrymans var. | |
| 165 | 166 | [symbol] P_001215222. [symbol] | conserved hypothetical protein [Aspergillus | LIGE |
| | | | terreus NIH2624] >gb\|EAU33805.1\|conserved | |
| | | | hypothetical protein [Aspergillus terreus | |
| 167 | 168 | [symbol] P_001823934. [symbol] | hypothetical protein AOR_1_322094 [Aspergillus | LIGE |
| | | | oryzae RIB40] >dbj\|BAE62801.1\|unnamed | |
| | | | protein product [Aspergillus oryzae RIB40] | |
| 169 | 170 | [symbol] P_001839188. [symbol] | hypothetical protein CC1G_07903 [Coprinopsis | LIGE |
| | | | cinerea okayama7#130] >gb\|EAU82621.1\| | |
| | | | hypothetical protein CC1G_07903 [Coprinopsis | |
| 171 | 172 | [symbol] P_001885678. [symbol] | predicted protein [Laccaria bicolor S238N-H82] | LIGE |
| | | | >gb\|EDR03530.1\|predicted protein [Laccaria | |
| | | | bicolor S238N-H82] | |
| 173 | 174 | [symbol] P_002152364. [symbol] | conserved hypothetical protein [Penicillium | LIGE |
| | | | marneffei ATCC 18224] >gb\|EEA19427.1\| | |
| | | | conserved hypothetical protein [Penicillium | |
| 175 | 176 | [symbol] P_002380998. [symbol] | conserved hypothetical protein [Aspergillus | LIGE |
| | | | flavus NRRL3357] >gb\|EED49097.1\|conserved | |
| | | | hypothetical protein [Aspergillus flavus | |
| 177 | 178 | [symbol] P_002392962. [symbol] | hypothetical protein MPER_07394 | LIGE |
| | | | [Moniliophthora perniciosa FA553] | |
| | | | >gb\|EEB93892.1\|hypothetical protein | |
| 179 | 180 | [symbol] P_002468854. [symbol] | predicted protein [Postia placenta Mad-698-R] | LIGE |
| | | | >gb\|EED86077.1\|predicted protein [Postia | |
| | | | placenta Mad-698-R] | |
| 181 | 182 | [symbol] P_002472522. [symbol] | predicted protein [Postia placenta Mad-698-R] | LIGE |
| | | | >gb\|EED82308.1\|predicted protein [Postia | |
| | | | placenta Mad-698-R] | |
| 183 | 184 | [symbol] P_002557398. [symbol] | Pc12g05530 [Penicillium chrysogenum | LIGE |
| | | | Wisconsin 54-1255] >emb\|CAP80180.1\| | |
| | | | Pc12g05530 [Penicillium chrysogenum | |
| 185 | 186 | [symbol] P_003026159. [symbol] | hypothetical protein SCHCODRAFT_12387 | LIGE |
| | | | [Schizophyllum commune H4-8] | |
| | | | >gb\|EFI91256.1\|hypothetical protein | |
| 187 | 188 | [symbol] P_003028923. [symbol] | hypothetical protein SCHCODRAFT_111982 | LIGE |
| | | | [Schizophyllum commune H4-8] | |
| | | | >gb\|EFI94020.1\|hypothetical protein | |
| 189 | 190 | [symbol] P_003890246. [symbol] | Glutathione S-transferase domain-containing | LIGE |
| | | | protein [Cyanothece sp. PCC 7822] | |
| | | | >gb\|ADN16971.1\|Glutathione S-transferase | |
| 191 | 192 | [symbol] P_003896657. [symbol] | glutathione S-transferase-like [Halomonas | LIGE |
| | | | elongata DSM 2581] >emb\|CBV41472.1\| | |
| | | | glutathione S-transferase-like [Halomonas | |
| 193 | 194 | [symbol] P_003980382. [symbol] | glutathione S-transferase [Achromobacter | LIGE |
| | | | xylosoxidans A8] >gb\|ADP17667.1\|glutathione | |
| | | | S-transferase, N-terminal domain protein 4 | |
| 195 | 196 | [symbol] P_004110838. [symbol] | glutathione S-transferase domain-containing | LIGE |
| | | | protein [Rhodopseudomonas palustris DX-1] | |
| | | | >gb\|ADU46105.1\|Glutathione S-transferase | |
| | | | domain [Rhodopseudomonas palustris DX-1] | |
| 197 | 198 | [symbol] P_004143867. [symbol] | glutathione S-transferase [Mesorhizobium ciceri | LIGE |
| | | | biovar biserrulae WSM1271] >gb\|ADV13817.1\| | |
| | | | Glutathione S-transferase domain | |
| | | | [Mesorhizobium ciceri biovar biserrulae | |
| 199 | 200 | ZP_01102591. [symbol] | conserved hypothetical protein [Congregibacter | LIGE |
| | | | litoralis KT71] >gb\|EAQ98305.1\|conserved | |
| | | | hypothetical protein [Congregibacter litoralis | |
| 201 | 202 | AAA87183.1 | auxin-induced protein [Vigna radiata] | LIGE |
| 203 | 204 | AAG34797.1 | glutathione S-transferase GST 7 [Glycine max] | LIGE |
| 205 | 206 | AAO69664.1 | glutathione S-transferase [Phaseolus acutifolius] | LIGE |
| 207 | 208 | ACU24385.1 | unknown [Glycine max] | LIGE |
| 209 | 210 | ADP99065.1 | glutathione S-transferase [Marinobacter | LIGE |
| 211 | 212 | ADY82158.1 | putative glutathione S-transferase [Acinetobacter | LIGE |
| | | | calcoaceticus PHEA-2] | |
| 213 | 214 | BAA77215.1 | beta-etherase [Sphingomonas paucimobilis] | LIGE |
| 215 | 216 | [symbol] P_001839584. [symbol] | hypothetical protein CC1G_12612 [Coprinopsis | LIGE |
| | | | cinerea okayama7#130] >gb\|EAU82225.1\| | |
| | | | hypothetical protein CC1G_12612 [Coprinopsis | |
| 217 | 218 | [symbol] P_002336443. [symbol] | predicted protein [Populus trichocarpa] | LIGE |
| | | | >gb\|EEE73479.1\|predicted protein [Populus | |
| 219 | 220 | [symbol] P_003028624. [symbol] | hypothetical protein SCHCODRAFT_59314 | LIGE |
| | | | [Schizophyllum commune H4-8] | |
| | | | >gb\|EFI93721.1\|hypothetical protein | |
| 221 | 222 | XP_456365.1 | DEHA2A00660p [Debaryomyces hansenii | LIGE |
| | | | CBS767] >emb\|CAG84310.1\|DEHA2A00660p | |
| | | | [Debaryomyces hansenii] | |
| 223 | 224 | XP_572781.1 | hypothetical protein [Cryptococcus neoformans | LIGE |
| | | | var. neoformans JEC21] >ref\|XP_773999.1\| | |
| | | | hypothetical protein CNBH0460 [Cryptococcus | |
| | | | neoformans var. neoformans B-3501A] | |
| | | | >gb\|EAL19352.1\|hypothetical protein | |
| | | | CNBH0460 [Cryptococcus neoformans var. | |
| | | | neoformans B-3501A] >gb\|AAW45474.1\| | |
| | | | [symbol] | |
| 225 | 226 | [symbol] P_001236206. [symbol] | glutathione S-transferase domain-containing | LIGE |
| | | | protein [Acidiphilium cryptum JF-5] | |
| | | | >gb\|ABQ32287.1\|Glutathione S-transferase, N- | |
| | | | terminal domain protein [Acidiphilium cryptum JF [symbol] | |
| 227 | 228 | [symbol] P_001237901. [symbol] | putative glutathione S-transferase | LIGE |
| | | | [Bradyrhizobium sp. BTAi1] >gb\|ABQ33995.1\| | |
| | | | putative glutathione S-transferase (GST) | |
| 229 | 230 | [symbol] P_001262153. [symbol] | hypothetical protein Swit_1652 [Sphingomonas | LIGE |
| | | | wittichii RW1] >gb\|ABQ68015.1\|hypothetical | |
| | | | protein Swit_1652 [Sphingomonas wittichii RW1] | |
| 231 | 232 | [symbol] P_001326465. [symbol] | glutathione S-transferase domain-containing | LIGE |
| | | | protein [Sinorhizobium medicae WSM419] | |
| | | | >gb\|ABR59630.1\|Glutathione S-transferase | |
| | | | domain [Sinorhizobium medicae WSM419] | |
| 233 | 234 | [symbol] P_001413220. [symbol] | glutathione S-transferase domain-containing | LIGE |
| | | | protein [Parvibaculum lavamentivorans DS-1] | |
| | | | >gb\|ABS63563.1\|Glutathione S-transferase | |
| | | | domain [Parvibaculum lavamentivorans DS-1] | |
| 235 | 236 | [symbol] P_001526182. [symbol] | glutathione S-transferase [Azorhizobium | LIGE |
| | | | caulinodans ORS 571] >dbj\|BAF89264.1\| | |
| | | | glutathione S-transferase [Azorhizobium | |
| 237 | 238 | YP_171459.1 | glutathione S-transferase [Synechococcus | LIGE |
| | | | elongatus PCC 6301] >ref\|YP_399807.1\| | |
| | | | glutathione S-transferase [Synechococcus | |
| | | | elongatus PCC 7942] >dbj\|BAD78939.1\| | |
| | | | glutathione S-transferase [Synechococcus | |
| | | | elongatus PCC 6301] >gb\|ABB56820.1\| | |
| 239 | 240 | YP_322424.1 | glutathione S-transferase-like protein [Anabaena | LIGE |
| | | | variabilis ATCC 29413] >gb\|ABA21529.1\| | |
| | | | Glutathione S-transferase-like protein | |
| 241 | 242 | ZP_01625805. [symbol] | glutathione S-transferase, putative [marine | LIGE |
| | | | gamma proteobacterium HTCC2080] | |
| | | | >gb\|EAW41324.1\|glutathione S-transferase, | |
| | | | putative [marine gamma proteobacterium | |
| 243 | 244 | ZP_01631145. [symbol] | Glutathione S-transferase-like protein [Nodularia | LIGE |
| | | | spumigena CCY9414] >gb\|EAW44220.1\| | |
| | | | Glutathione S-transferase-like protein [Nodularia | |
| 245 | 246 | ZP_06057261. [symbol] | glutathione S-transferase [Acinetobacter | LIGE |
| | | | calcoaceticus RUH2202] >gb\|EEY78560.1\| | |
| | | | glutathione S-transferase [Acinetobacter | |

[symbol]

indicates data missing or illegible when filed

TABLE 17

| PROTEIN | GENE | GENBANK | | |
|---|---|---|---|---|
| SEQ ID | SEQ ID | ACCESSION | | |
| NO: | NO: | NO: | DESCRIPTION: | TYPE |
| 247 | 248 | AAB65163.1 | glutathione S-transferase, class-phi | LigF |
| | | | [Solanum commersonii] | |
| 249 | 250 | AAG34850.1 | glutathione S-transferase GST 42 [Zea | LigF |
| | | | mays] | |
| 251 | 252 | AAK98535.1 | putative glutathione S-transferase | LigF |
| | | | OsGSTU7 [Oryza sativa Japonica | |
| | | | Group] | |
| 253 | 254 | AAL61612.1 | glutathione S-transferase [Allium cepa] | LigF |
| 255 | 256 | ABE86679.1 | Intracellular chloride channel [Medicago | LigF |
| | | | truncatula] | |
| 257 | 258 | ABE86683.1 | Intracellular chloride channel [Medicago | LigF |
| | | | truncatula] | |
| 259 | 260 | ABQ96853.1 | glutathione S-transferase [Solanum | LigF |
| | | | tuberosum] | |
| 261 | 262 | ACF15452.1 | glutathione-S-transferase | LigF |
| | | | [Phanerochaete chrysosporium] | |
| 263 | 264 | ACG44597.1 | glutathione S-transferase GSTU6 [Zea | LigF |
| | | | mays] | |
| 265 | 266 | ACJ86045.1 | unknown [Medicago truncatula] | LigF |
| 267 | 268 | ACO15091.1 | Probable maleylacetoacetate isomerase | LigF |
| | | | 2 [Caligus clemensi] | |
| 269 | 270 | ADB11335.1 | phi class glutathione transferase GSTF7 | LigF |
| | | | [Populus trichocarpa] | |
| 271 | 272 | BAB70616.1 | glutathione S-transferase [Medicago | LigF |
| | | | sativa] | |
| 273 | 274 | BAF56180.1 | glutathione S-transferase [Allium cepa] | LigF |
| 275 | 276 | BAJ90004.1 | predicted protein [Hordeum vulgare | LigF |
| | | | subsp. vulgare] >dbj\|BAJ99460.1\| | |
| | | | predicted protein [Hordeum vulgare | |
| | | | subsp. vulgare] | |
| 277 | 278 | CAI51314.2 | glutathione S-transferase GST1 | LigF |
| | | | [Capsicum chinense] | |
| 279 | 280 | EAY79299.1 | hypothetical protein OsI_34425 [Oryza | LigF |
| | | | sativa Indica Group] | |
| 281 | 282 | EAZ16758.1 | hypothetical protein OsJ_32234 [Oryza | LigF |
| | | | sativa Japonica Group] | |
| 283 | 284 | EEC67342.1 | hypothetical protein OsI_34397 [Oryza | LigF |
| | | | sativa Indica Group] | |
| 285 | 286 | EFV87279.1 | glutathione S-transferase | LigF |
| | | | [Achromobacter xylosoxidans C54] | |
| 287 | 288 | EGN92742.1 | hypothetical protein | LigF |
| | | | SERLA73DRAFT_190579 [Serpula | |
| | | | lacrymans var. lacrymans S7.3] | |
| | | | >gb\|EGO26403.1\|hypothetical protein | |
| | | | SERLADRAFT_463437 [Serpula | |
| | | | lacrymans var. lacrymans S7.9] | |
| 289 | 290 | EGU75635.1 | hypothetical protein FOXB_13869 | LigF |
| | | | [Fusarium oxysporum Fo5176] | |
| 291 | 292 | NP_001065115.1 | Os10g0525600 [Oryza sativa Japonica | LigF |
| | | | Group] >gb\|AAM12493.1\|AC074232_20 | |
| | | | putative glutathione S-transferase [Oryza | |
| | | | sativa Japonica Group] | |
| | | | >dbj\|BAF27029.1\|Os10g0525600 | |
| | | | [Oryza sativa Japonica Group] | |
| 293 | 294 | NP_001065118.1 | Os10g0527400 [Oryza sativa Japonica | LigF |
| | | | Group] >gb\|AAM12310.1\|AC091680_11 | |
| | | | putative glutathione S-transferase [Oryza | |
| | | | sativa Japonica Group] | |
| | | | >gb\|AAM12478.1\|AC074232_5 putative | |
| | | | glutathione S-transferase [Oryza sativa | |
| | | | Japonica Group] >gb\|AAP54729.1\| | |
| | | | glutathione S-transferase GSTU6, | |
| | | | putative, expressed [Oryza sativa | |
| | | | Japonica Group] >dbj\|BAF27032.1\| | |
| | | | Os10g0527400 [Oryza sativa Japonica | |
| | | | Group] >gb\|EEE51298.1\|hypothetical | |
| | | | protein OsJ_32225 [Oryza sativa | |
| | | | Japonica Group] | |
| 295 | 296 | NP_001065126.1 | Os10g0529300 [Oryza sativa Japonica | LigF |
| | | | Group] >gb\|AAK98546.1\|AF402805_1 | |
| | | | putative glutathione S-transferase | |
| | | | OsGSTU18 [Oryza sativa Japonica | |
| | | | Group] >gb\|AAM12302.1\|AC091680_3 | |
| | | | putative glutathione S-transferase [Oryza | |
| | | | sativa Japonica Group] | |
| | | | >gb\|AAM94529.1\|putative glutathione S- | |
| | | | transferase [Oryza sativa Japonica | |
| | | | Group] >gb\|AAP54753.1\|glutathione S- | |
| | | | transferase GSTU6, putative, expressed | |
| | | | [Oryza sativa Japonica Group] | |
| | | | >dbj\|BAF27040.1\|Os10g0529300 | |
| | | | [Oryza sativa Japonica Group] | |
| | | | >gb\|EAY79288.1\|hypothetical protein | |
| | | | OsI_34414 [Oryza sativa Indica Group] | |
| | | | >dbj\|BAG87628.1\|unnamed protein | |
| | | | product [Oryza sativa Japonica Group] | |
| | | | >dbj\|BAG97643.1\|unnamed protein | |
| | | | product [Oryza sativa Japonica Group] | |
| | | | >dbj\|BAG87189.1\|unnamed protein | |
| | | | product [Oryza sativa Japonica Group] | |
| 297 | 298 | NP_001065132.1 | Os10g0529900 [Oryza sativa Japonica | LigF |
| | | | Group] >gb\|AAM12331.1\|AC091680_32 | |
| | | | putative glutathione S-transferase [Oryza | |
| | | | sativa Japonica Group] | |
| | | | >gb\|AAM94517.1\|putative glutathione S- | |
| | | | transferase [Oryza sativa Japonica | |
| | | | Group] >gb\|AAP54759.1\|glutathione S- | |
| | | | transferase GSTU6, putative [Oryza | |
| | | | sativa Japonica Group] | |
| | | | >dbj\|BAF27046.1\|Os10g0529900 | |
| | | | [Oryza sativa Japonica Group] | |
| | | | >gb\|EAZ16763.1\|hypothetical protein | |
| | | | OsJ_32239 [Oryza sativa Japonica | |
| | | | Group] | |
| 299 | 300 | NP_001105627.1 | LOC542632 [Zea mays] | LigF |
| | | | >gb\|AAG34835.1\|AF244692_1 | |
| | | | glutathione S-transferase GST 27 [Zea | |
| | | | mays] >gb\|ACF85142.1\|unknown [Zea | |
| | | | mays] | |
| 301 | 302 | NP_001152229.1 | glutathione S-transferase GSTU6 [Zea | LigF |
| | | | mays] >gb\|ACG46501.1\|glutathione S- | |
| | | | transferase GSTU6 [Zea mays] | |
| 303 | 304 | NP_384409.1 | putative glutathione S-transferase | LigF |
| | | | protein [Sinorhizobium meliloti 1021] | |
| | | | >ref\|YP_004550950.1\|glutathione S- | |
| | | | transferase domain-containing protein | |
| | | | [Sinorhizobium meliloti AK83] | |
| | | | >emb\|CAC41740.1\|Putative glutathione | |
| | | | S-transferase [Sinorhizobium meliloti | |
| | | | 1021] >gb\|AEG06303.1\|Glutathione S- | |
| | | | transferase domain protein | |
| | | | [Sinorhizobium meliloti BL225C] | |
| | | | >gb\|AEG55336.1\|Glutathione S- | |
| | | | transferase domain protein | |
| | | | [Sinorhizobium meliloti AK83] | |
| | | | >gb\|AEH81005.1\|putative glutathione S- | |
| | | | transferase protein [Sinorhizobium | |
| | | | meliloti SM11] | |
| 305 | 306 | XP_001555922.1 | hypothetical protein BC1G_05597 | LigF |
| | | | [Botryotinia fuckeliana B05.10] | |
| | | | >gb\|EDN24875.1\|hypothetical protein | |
| | | | BC1G_05597 [Botryotinia fuckeliana | |
| | | | B05.10] | |
| 307 | 308 | XP_001805855.1 | hypothetical protein SNOG_15716 | LigF |
| | | | [Phaeosphaeria nodorum SN15] | |
| | | | >gb\|EAT76811.2\|hypothetical protein | |
| | | | SNOG_15716 [Phaeosphaeria nodorum | |
| | | | SN15] | |
| 309 | 310 | XP_002321320.1 | predicted protein [Populus trichocarpa] | LigF |
| | | | >gb\|EEE99635.1\|predicted protein | |
| | | | [Populus trichocarpa] | |
| 311 | 312 | XP_002455784.1 | hypothetical protein | LigF |
| | | | SORBIDRAFT_03g025210 [Sorghum | |
| | | | bicolor] >gb\|EES00904.1\|hypothetical | |
| | | | protein SORBIDRAFT_03g025210 | |
| | | | [Sorghum bicolor] | |
| 313 | 314 | XP_002467606.1 | hypothetical protein | LigF |
| | | | SORBIDRAFT_01g030860 [Sorghum | |
| | | | bicolor] >gb\|EER94604.1\|hypothetical | |
| | | | protein SORBIDRAFT_01g030860 | |
| | | | [Sorghum bicolor] | |
| 315 | 316 | XP_002734706.1 | PREDICTED: ganglioside-induced | LigF |
| | | | differentiation-associated protein 1-like | |
| | | | [Saccoglossus kowalevskii] | |
| 317 | 318 | XP_002734707.1 | PREDICTED: ganglioside-induced | LigF |
| | | | differentiation-associated protein 1-like | |
| | | | [Saccoglossus kowalevskii] | |
| 319 | 320 | XP_002737947.1 | PREDICTED: Glutathione S-Transferase | LigF |
| | | | family member (gst-42)-like | |
| | | | [Saccoglossus kowalevskii] | |
| 321 | 322 | XP_002989538.1 | hypothetical protein | LigF |
| | | | SELMODRAFT_184606 [Selaginella | |
| | | | moellendorffii] >gb\|EFJ09414.1\| | |
| | | | hypothetical protein | |
| | | | SELMODRAFT_184606 [Selaginella | |
| | | | moellendorffii] | |
| 323 | 324 | XP_003146962.1 | glutathione S-transferase domain- | LigF |
| | | | containing protein [Loa loa] | |
| | | | >gb\|EFO17107.1\|glutathione S- | |
| | | | transferase domain-containing protein | |
| | | | [Loa loa] | |
| 325 | 326 | YP_001187408.1 | glutathione S-transferase domain- | LigF |
| | | | containing protein [Pseudomonas | |
| | | | mendocina ymp] >gb\|ABP84676.1\| | |
| | | | Glutathione S-transferase, N-terminal | |
| | | | domain protein [Pseudomonas | |
| | | | mendocina ymp] | |
| 327 | 328 | YP_001239734.1 | glutathione S-transferase domain- | LigF |
| | | | containing protein [Bradyrhizobium sp. | |
| | | | BTAi1] >gb\|ABQ35828.1\|putative | |
| | | | glutathione S-transferase enzyme with | |
| | | | thioredoxin-like domain [Bradyrhizobium | |
| | | | sp. BTAi1] | |
| 329 | 330 | YP_001261939.1 | glutathione S-transferase domain- | LigF |
| | | | containing protein [Sphingomonas | |
| | | | wittichii RW1] >gb\|ABQ67801.1\| | |
| | | | Glutathione S-transferase, N-terminal | |
| | | | domain [Sphingomonas wittichii RW1] | |
| 331 | 332 | YP_001263066.1 | glutathione S-transferase domain- | LigF |
| | | | containing protein [Sphingomonas | |
| | | | wittichii RW1] >gb\|ABQ68928.1\| | |
| | | | Glutathione S-transferase, N-terminal | |
| | | | domain [Sphingomonas wittichii RW1] | |
| 333 | 334 | YP_001414366.1 | glutathione S-transferase domain- | LigF |
| | | | containing protein [Parvibaculum | |
| | | | lavamentivorans DS-1] | |
| | | | >gb\|ABS64709.1\|Glutathione S- | |
| | | | transferase domain [Parvibaculum | |
| | | | lavamentivorans DS-1] | |
| 335 | 336 | YP_001414838.1 | maleylacetoacetate isomerase | LigF |
| | | | [Parvibaculum lavamentivorans DS-1] | |
| | | | >gb\|ABS65181.1\|maleylacetoacetate | |
| | | | isomerase [Parvibaculum | |
| | | | lavamentivorans DS-1] | |
| 337 | 338 | YP_001684291.1 | glutathione S-transferase domain- | LigF |
| | | | containing protein [Caulobacter sp. K31] | |
| | | | >gb\|ABZ71793.1\|Glutathione S- | |
| | | | transferase domain [Caulobacter sp. | |
| | | | K31] | |
| 339 | 340 | YP_001770584.1 | glutathione S-transferase domain- | LigF |
| | | | containing protein [Methylobacterium sp. | |
| | | | 4-46] >gb\|ACA18150.1\|Glutathione S- | |
| | | | transferase domain [Methylobacterium | |
| | | | sp. 4-46] | |
| 341 | 342 | YP_002828116.1 | predicted glutathione S-transferase | LigF |
| | | | protein [Sinorhizobium fredii NGR234] | |
| | | | >gb\|ACP27363.1\|predicted glutathione | |
| | | | S-transferase protein [Sinorhizobium | |
| | | | fredii NGR234] | |
| 343 | 344 | YP_003593122.1 | glutathione S-transferase domain- | LigF |
| | | | containing protein [Caulobacter segnis | |
| | | | ATCC 21756] >gb\|ADG10504.1\| | |
| | | | Glutathione S-transferase domain | |
| | | | protein [Caulobacter segnis ATCC | |
| | | | 21756] | |
| 345 | 346 | YP_003930867.1 | glutathione S-transferase [Pantoea | LigF |
| | | | vagans C9-1] >gb\|ADO09418.1\| | |
| | | | Glutathione S-transferase [Pantoea | |
| | | | vagans C9-1] | |
| 347 | 348 | YP_004434596.1 | Glutathione S-transferase domain | LigF |
| | | | protein [Glaciecola agarilytica 4H-3- | |
| | | | 7 + YE-5] >gb\|AEE23328.1\|Glutathione S- | |
| | | | transferase domain protein [Glaciecola | |
| | | | sp. 4H-3-7 + YE-5] | |
| 349 | 350 | YP_004620883.1 | glutathione S-transferase [Ramlibacter | LigF |
| | | | tataouinensis TTB310] | |
| | | | >gb\|AEG94864.1\|glutathione S- | |
| | | | transferase-like protein [Ramlibacter | |
| | | | tataouinensis TTB310] | |
| 351 | 352 | YP_067874.1 | glutathione S-transferase family protein | LigF |
| | | | [Aeromonas punctata] | |
| | | | >emb\|CAG15111.1\|glutathione S- | |
| | | | transferase family protein [Aeromonas | |
| | | | caviae] | |
| 353 | 354 | YP_168502.1 | glutathione S-transferase, putative | LigF |
| | | | [Ruegeria pomeroyi DSS-3] | |
| | | | >gb\|AAV96533.1\|glutathione S- | |
| | | | transferase, putative [Ruegeria pomeroyi | |
| | | | DSS-3] | |
| 355 | 356 | YP_339058.1 | glutathione S-transferase | LigF |
| | | | [Pseudoalteromonas haloplanktis | |
| | | | TAC125] >emb\|CAI85615.1\|putative | |
| | | | glutathione S-transferase | |
| | | | [Pseudoalteromonas haloplanktis | |
| | | | TAC125] | |
| 357 | 358 | YP_612204.1 | glutathione S-transferase-like [Ruegeria | LigF |
| | | | sp. TM1040] >gb\|ABF62942.1\| | |
| | | | glutathione S-transferase-like protein | |
| | | | [Ruegeria sp. TM1040] | |
| 359 | 360 | ZP_00954574.1 | glutathione S-transferase family protein | LigF |
| | | | [Sulfitobacter sp. EE-36] | |
| | | | >ref\|ZP_00961889.1\|glutathione S- | |
| | | | transferase family protein [Sulfitobacter | |
| | | | sp. NAS-14.1] >gb\|EAP81303.1\| | |
| | | | glutathione S-transferase family protein | |
| | | | [Sulfitobacter sp. NAS-14.1] | |
| | | | >gb\|EAP85807.1\|glutathione S- | |
| | | | transferase family protein [Sulfitobacter | |
| | | | sp. EE-36] | |
| 361 | 362 | ZP_01165363.1 | maleylacetoacetate isomerase | LigF |
| | | | [Oceanospirillum sp. MED92] | |
| | | | >gb\|EAR62715.1\|maleylacetoacetate | |
| | | | isomerase [Oceanospirillum sp. MED92] | |
| 363 | 364 | ZP_01881157.1 | glutathione S-transferase, putative | LigF |
| | | | [Roseovarius sp. TM1035] | |
| | | | >gb\|EDM30676.1\|glutathione S- | |
| | | | transferase, putative [Roseovarius sp. | |
| | | | TM1035] | |
| 365 | 366 | ZP_03523367.1 | Glutathione S-transferase domain | LigF |
| | | | [Rhizobium etli GR56] | |
| 367 | 368 | ZP_04614975.1 | Glutathione S-transferase GST-6.0 | LigF |
| | | | [Yersinia ruckeri ATCC 29473] | |
| | | | >gb\|EEQ00521.1\|Glutathione S- | |
| | | | transferase GST-6.0 [Yersinia ruckeri | |
| | | | ATCC 29473] | |
| 369 | 370 | ZP_05125190.1 | glutathione S-transferase, N-terminal | LigF |
| | | | domain protein [Rhodobacteraceae | |
| | | | bacterium KLH11] >gb\|EEE36118.1\| | |
| | | | glutathione S-transferase, N-terminal | |
| | | | domain protein [Rhodobacteraceae | |
| | | | bacterium KLH11] | |
| 371 | 372 | ZP_05786193.1 | glutathione S-transferase [Silicibacter | LigF |
| | | | lacuscaerulensis ITI-1157] | |
| | | | >gb\|EEX09309.1\|glutathione S- | |
| | | | transferase [Silicibacter lacuscaerulensis | |
| | | | ITI-1157] | |
| 373 | 374 | ZP_08264339.1 | maleylacetoacetate isomerase | LigF |
| | | | [Asticcacaulis biprosthecum C19] | |
| | | | >gb\|EGF90974.1\|maleylacetoacetate | |
| | | | isomerase [Asticcacaulis biprosthecum | |
| | | | C19] | |
| 375 | 376 | ZP_08630058.1 | glutathione S-transferase | LigF |
| | | | [Bradyrhizobiaceae bacterium SG-6C] | |
| | | | >gb\|EGP07427.1\|glutathione S- | |
| | | | transferase [Bradyrhizobiaceae | |
| | | | bacterium SG-6C] | |
| 377 | 378 | AAG34806.1 | glutathione S-transferase GST 16 | LigF |
| | | | [Glycine max] | |
| 379 | 380 | AAQ02687.1 | tau class GST protein 3 [Oryza sativa | LigF |
| | | | Indica Group] >gb\|EAY79295.1\| | |
| | | | hypothetical protein OsI_34421 [Oryza | |
| | | | sativa Indica Group] >emb\|CAZ68078.1\| | |
| | | | glutathione S-transferase [Oryza sativa | |
| | | | Indica Group] | |
| 381 | 382 | ADV56298.1 | Glutathione S-transferase domain | LigF |
| | | | protein [Shewanella putrefaciens 200] | |
| 383 | 384 | BAB70616.1 | glutathione S-transferase [Medicago | LigF |
| | | | sativa] | |
| 385 | 386 | BAJ94610.1 | predicted protein [Hordeum vulgare | LigF |
| | | | subsp. vulgare] | |
| 387 | 388 | CAN68934.1 | hypothetical protein VITISV_002763 | LigF |
| | | | [Vitis vinifera] | |
| 389 | 390 | CBW26056.1 | putative glutathione S-transferase | LigF |
| | | | [Bacteriovorax marinus SJ] | |
| 391 | 392 | EFW18159.1 | glutathione S-transferase [Coccidioides | LigF |
| | | | posadasii str. Silveira] | |
| 393 | 394 | EGF84337.1 | hypothetical protein | LigF |
| | | | BATDEDRAFT_85058 | |
| | | | [Batrachochytrium dendrobatidis JAM81] | |
| 395 | 396 | NP_001065124.1 | Os10g0528400 [Oryza sativa Japonica | LigF |
| | | | Group] >gb\|AAG32472.1\|AF309379_1 | |
| | | | putative glutathione S-transferase | |
| | | | OsGSTU3 [Oryza sativa Japonica | |
| | | | Group] >gb\|AAM12325.1\|AC091680_26 | |
| | | | putative glutathione S-transferase [Oryza | |
| | | | sativa Japonica Group] | |
| | | | >gb\|AAM94544.1\|putative glutathione S- | |
| | | | transferase [Oryza sativa Japonica | |
| | | | Group] >gb\|AAP54745.1\|glutathione S- | |
| | | | transferase GSTU6, putative, expressed | |
| | | | [Oryza sativa Japonica Group] | |
| | | | >dbj\|BAF27038.1\|Os10g0528400 | |
| | | | [Oryza sativa Japonica Group] | |
| | | | >gb\|EAZ16756.1\|hypothetical protein | |
| | | | OsJ_32232 [Oryza sativa Japonica | |
| | | | Group] | |
| 397 | 398 | NP_191835.1 | Glutathione S-transferase-like protein | LigF |
| | | | [Arabidopsis thaliana] | |
| | | | >emb\|CAB83126.1\|Glutathione | |
| | | | transferase III-like protein [Arabidopsis | |
| | | | thaliana] >gb\|AEE80388.1\|Glutathione | |
| | | | S-transferase-like protein [Arabidopsis | |
| | | | thaliana] | |
| 399 | 400 | NP_717190.1 | glutathione S-transferase family protein | LigF |
| | | | [Shewanella oneidensis MR-1] | |
| | | | >gb\|AAN54634.1\|AE015603_8 | |
| | | | glutathione S-transferase family protein | |
| | | | [Shewanella oneidensis MR-1] | |
| 401 | 402 | NP_769143.1 | glutathione S-transferase | LigF |
| | | | [Bradyrhizobium japonicum USDA 110] | |
| | | | >dbj\|BAC47768.1\|glutathione S- | |
| | | | transferase [Bradyrhizobium japonicum | |
| | | | USDA 110] | |
| 403 | 404 | NP_900642.1 | glutathione transferase zeta 1 | LigF |
| | | | [Chromobacterium violaceum ATCC | |
| | | | 12472] >gb\|AAQ58646.1\|probable | |
| | | | glutathione transferase zeta 1 | |
| | | | [Chromobacterium violaceum ATCC | |
| | | | 12472] | |
| 405 | 406 | XP_001246353.1 | glutathione S-transferase [Coccidioides | LigF |
| | | | immitis RS] | |
| 407 | 408 | XP_002171087.1 | PREDICTED: similar to glutathione S- | LigF |
| | | | transferase [Hydra magnipapillata] | |
| 409 | 410 | XP_002263386.1 | PREDICTED: hypothetical protein [Vitis | LigF |
| | | | vinifera] >emb\|CBI32223.3\|unnamed | |
| | | | protein product [Vitis vinifera] | |
| 411 | 412 | XP_002263424.1 | PREDICTED: hypothetical protein [Vitis | LigF |
| | | | vinifera] >emb\|CBI32222.3\|unnamed | |
| | | | protein product [Vitis vinifera] | |
| 413 | 414 | XP_002272099.1 | PREDICTED: hypothetical protein | LigF |
| | | | isoform 2 [Vitis vinifera] | |
| 415 | 416 | XP_002527848.1 | glutathione s-transferase, putative | LigF |
| | | | [Ricinus communis] >gb\|EEF34551.1\| | |
| | | | glutathione s-transferase, putative | |
| | | | [Ricinus communis] | |
| 417 | 418 | XP_002786341.1 | Glutathione S-transferase A, putative | LigF |
| | | | [Perkinsus marinus ATCC 50983] | |
| | | | >gb\|EER18137.1\|Glutathione S- | |
| | | | transferase A, putative [Perkinsus | |
| | | | marinus ATCC 50983] | |
| 419 | 420 | XP_003066789.1 | Glutathione S-transferase, putative | LigF |
| | | | [Coccidioides posadasii C735 delta | |
| | | | SOWgp] >gb\|EER24644.1\|Glutathione | |
| | | | S-transferase, putative [Coccidioides | |
| | | | posadasii C735 delta SOWgp] | |
| 421 | 422 | XP_970577.1 | PREDICTED: similar to ganglioside- | LigF |
| | | | induced differentiation-associated- | |
| | | | protein 1 [Tribolium castaneum] | |
| | | | >gb\|EFA00477.1\|hypothetical protein | |
| | | | TcasGA2_TC003336 [Tribolium | |
| | | | castaneum] | |
| 423 | 424 | YP_001263559.1 | glutathione S-transferase domain- | LigF |
| | | | containing protein [Sphingomonas | |
| | | | wittichii RW1] >gb\|ABQ69421.1\| | |
| | | | Glutathione S-transferase, N-terminal | |
| | | | domain [Sphingomonas wittichii RW1] | |
| 425 | 426 | YP_001503032.1 | glutathione S-transferase domain- | LigF |
| | | | containing protein [Shewanella pealeana | |
| | | | ATCC 700345] >gb\|ABV88497.1\| | |
| | | | Glutathione S-transferase domain | |
| | | | [Shewanella pealeana ATCC 700345] | |
| 427 | 428 | YP_001516981.1 | glutathione S-transferase II | LigF |
| | | | [Acaryochloris marina MBIC11017] | |
| | | | >gb\|ABW27665.1\|glutathione S- | |
| | | | transferase II [Acaryochloris marina | |
| | | | MBIC11017] | |
| 429 | 430 | YP_001615392.1 | glutathione S-transferase, [Sorangium | LigF |
| | | | cellulosum ‘So ce 56’] | |
| | | | >emb\|CAN94912.1\|glutathione S- | |
| | | | transferase, putative [Sorangium | |
| | | | cellulosum ‘So ce 56’] | |
| 431 | 432 | YP_001685556.1 | glutathione S-transferase domain- | LigF |
| | | | containing protein [Caulobacter sp. K31] | |
| | | | >gb\|ABZ73058.1\|Glutathione S- | |
| | | | transferase domain [Caulobacter sp. | |
| | | | K31] | |
| 433 | 434 | YP_001748054.1 | glutathione S-transferase domain- | LigF |
| | | | containing protein [Pseudomonas putida | |
| | | | W619] >gb\|ACA71685.1\|Glutathione S- | |
| | | | transferase domain [Pseudomonas | |
| | | | putida W619] | |
| 435 | 436 | YP_001804371.1 | glutathione S-transferase [Cyanothece | LigF |
| | | | sp. ATCC 51142] >gb\|ACB52305.1\| | |
| | | | glutathione S-transferase [Cyanothece | |
| | | | sp. ATCC 51142] | |
| 437 | 438 | YP_002007283.1 | glutathione s-transferase protein; gsta | LigF |
| | | | protein [Cupriavidus taiwanensis LMG | |
| | | | 19424] >emb\|CAQ71222.1\|putative | |
| | | | glutathione S-transferase protein; gstA | |
| | | | protein [Cupriavidus taiwanensis LMG | |
| | | | 19424] | |
| 439 | 440 | YP_002130812.1 | glutathione S-transferase | LigF |
| | | | [Phenylobacterium zucineum HLK1] | |
| | | | >gb\|ACG78383.1\|glutathione S- | |
| | | | transferase [Phenylobacterium zucineum | |
| | | | HLK1] | |
| 441 | 442 | YP_002220633.1 | glutathione S-transferase domain | LigF |
| | | | [Acidithiobacillus ferrooxidans ATCC | |
| | | | 53993] >ref\|YP_002426974.1\| | |
| | | | glutathione S-transferase | |
| | | | [Acidithiobacillus ferrooxidans ATCC | |
| | | | 23270] >gb\|ACH84426.1\|Glutathione S- | |
| | | | transferase domain [Acidithiobacillus | |
| | | | ferrooxidans ATCC 53993] | |
| | | | >gb\|ACK78121.1\|glutathione S- | |
| | | | transferase [Acidithiobacillus | |
| | | | ferrooxidans ATCC 23270] | |
| 443 | 444 | YP_002482418.1 | glutathione S-transferase domain- | LigF |
| | | | containing protein [Cyanothece sp. PCC | |
| | | | 7425] >gb\|ACL44057.1\|Glutathione S- | |
| | | | transferase domain protein [Cyanothece | |
| | | | sp. PCC 7425] | |
| 445 | 446 | YP_002543747.1 | glutathione S-transferase protein | LigF |
| | | | [Agrobacterium radiobacter K84] | |
| | | | >gb\|ACM25821.1\|glutathione S- | |
| | | | transferase protein [Agrobacterium | |
| | | | radiobacter K84] | |
| 447 | 448 | YP_002974739.1 | glutathione S-transferase domain protein | LigF |
| | | | [Rhizobium leguminosarum bv. trifolii | |
| | | | WSM1325] >gb\|ACS55200.1\| | |
| | | | Glutathione S-transferase domain | |
| | | | protein [Rhizobium leguminosarum bv. | |
| | | | trifolii WSM1325] | |
| 449 | 450 | YP_004065207.1 | glutathione transferase | LigF |
| | | | [Pseudoalteromonas sp. SM9913] | |
| | | | >gb\|ADT70298.1\|glutathione | |
| | | | transferase [Pseudoalteromonas sp. | |
| | | | SM9913] | |
| 451 | 452 | YP_004357179.1 | glutathione S-transferase [Pseudomonas | LigF |
| | | | brassicacearum subsp. brassicacearum | |
| | | | NFM421] >gb\|AEA72175.1\|putative | |
| | | | glutathione S-transferase [Pseudomonas | |
| | | | brassicacearum subsp. brassicacearum | |
| | | | NFM421] | |
| 453 | 454 | YP_004680920.1 | glutathione S-transferase [Cupriavidus | LigF |
| | | | necator N-1] >gb\|AEI79688.1\| | |
| | | | glutathione S-transferase [Cupriavidus | |
| | | | necator N-1] | |
| 455 | 456 | YP_468810.1 | glutathione S-transferase [Rhizobium etli | LigF |
| | | | CFN 42] >gb\|ABC90083.1\|glutathione S- | |
| | | | transferase protein [Rhizobium etli CFN | |
| | | | 42] | |
| 457 | 458 | YP_554040.1 | glutathione S-transferase [Burkholderia | LigF |
| | | | xenovorans LB400] >gb\|ABE34690.1\| | |
| | | | Glutathione S-transferase [Burkholderia | |
| | | | xenovorans LB400] | |
| 459 | 460 | YP_612103.1 | glutathione S-transferase-like [Ruegeria | LigF |
| | | | sp. TM1040] >gb\|ABF62841.1\| | |
| | | | glutathione S-transferase-like protein | |
| | | | [Ruegeria sp. TM1040] | |
| 461 | 462 | YP_735310.1 | glutathione S-transferase domain- | LigF |
| | | | containing protein [Shewanella sp. MR-4] | |
| | | | >gb\|ABI40253.1\|Glutathione S- | |
| | | | transferase, N-terminal domain protein | |
| | | | [Shewanella sp. MR-4] | |
| 463 | 464 | YP_747567.1 | glutathione S-transferase domain- | LigF |
| | | | containing protein [Nitrosomonas | |
| | | | eutropha C91] >gb\|ABI59602.1\| | |
| | | | Glutathione S-transferase, C-terminal | |
| | | | domain [Nitrosomonas eutropha C91] | |
| 465 | 466 | YP_757227.1 | maleylacetoacetate isomerase | LigF |
| | | | [Maricaulis maris MCS10] | |
| | | | >gb\|ABI66289.1\|maleylacetoacetate | |
| | | | isomerase [Maricaulis maris MCS10] | |
| 467 | 468 | YP_868399.1 | glutathione S-transferase domain- | LigF |
| | | | containing protein [Shewanella sp. ANA- | |
| | | | 3] >gb\|ABK46993.1\|Glutathione S- | |
| | | | transferase, N-terminal domain protein | |
| | | | [Shewanella sp. ANA-3] | |
| 469 | 470 | YP_870498.1 | glutathione S-transferase domain- | LigF |
| | | | containing protein [Shewanella sp. ANA- | |
| | | | 3] >gb\|ABK49092.1\|Glutathione S- | |
| | | | transferase, N-terminal domain protein | |
| | | | [Shewanella sp. ANA-3] | |
| 471 | 472 | YP_957711.1 | glutathione S-transferase domain- | LigF |
| | | | containing protein [Marinobacter | |
| | | | aquaeolei VT8] >gb\|ABM17524.1\| | |
| | | | Glutathione S-transferase, N-terminal | |
| | | | domain [Marinobacter aquaeolei VT8] | |
| 473 | 474 | YP_957873.1 | glutathione S-transferase domain- | LigF |
| | | | containing protein [Marinobacter | |
| | | | aquaeolei VT8] >gb\|ABM17686.1\| | |
| | | | Glutathione S-transferase, N-terminal | |
| | | | domain [Marinobacter aquaeolei VT8] | |
| 475 | 476 | YP_960793.1 | glutathione S-transferase domain- | LigF |
| | | | containing protein [Marinobacter | |
| | | | aquaeolei VT8] >gb\|ABM20606.1\| | |
| | | | Glutathione S-transferase, N-terminal | |
| | | | domain [Marinobacter aquaeolei VT8] | |
| 477 | 478 | YP_963418.1 | glutathione S-transferase domain- | LigF |
| | | | containing protein [Shewanella sp. W3- | |
| | | | 18-1] >gb\|ABM24864.1\|Glutathione S- | |
| | | | transferase, N-terminal domain | |
| | | | [Shewanella sp. W3-18-1] | |
| 479 | 480 | ZP_01000028.1 | glutathione S-transferase family protein | LigF |
| | | | [Oceanicola batsensis HTCC2597] | |
| | | | >gb\|EAQ02499.1\|glutathione S- | |
| | | | transferase family protein [Oceanicola | |
| | | | batsensis HTCC2597] | |
| 481 | 482 | ZP_01459182.1 | glutathione S-transferase [Stigmatella | LigF |
| | | | aurantiaca DW4/3-1] | |
| | | | >ref\|YP_003956548.1\|glutathione s- | |
| | | | transferase [Stigmatella aurantiaca | |
| | | | DW4/3-1] >gb\|EAU70026.1\|glutathione | |
| | | | S-transferase [Stigmatella aurantiaca | |
| | | | DW4/3-1] >gb\|ADO74721.1\|Glutathione | |
| | | | S-transferase [Stigmatella aurantiaca | |
| | | | DW4/3-1] | |
| 483 | 484 | ZP_02886014.1 | Glutathione S-transferase domain | LigF |
| | | | [Burkholderia graminis C4D1M] | |
| | | | >gb\|EDT08402.1\|Glutathione S- | |
| | | | transferase domain [Burkholderia | |
| | | | graminis C4D1M] | |
| 485 | 486 | ZP_04713937.1 | Glutathione S-transferase [Alteromonas | LigF |
| | | | macleodii ATCC 27126] | |
| 487 | 488 | ZP_05075049.1 | Glutathione S-transferase, N-terminal | LigF |
| | | | domain protein [Rhodobacterales | |
| | | | bacterium HTCC2083] >gb\|EDZ42709.1\| | |
| | | | Glutathione S-transferase, N-terminal | |
| | | | domain protein [Rhodobacteraceae | |
| | | | bacterium HTCC2083] | |
| 489 | 490 | ZP_05101428.1 | glutathione S-transferase protein | LigF |
| | | | [Roseobacter sp. GAI101] | |
| | | | >gb\|EEB85730.1\|glutathione S- | |
| | | | transferase protein [Roseobacter sp. | |
| | | | GAI101] | |
| 491 | 492 | ZP_05124402.1 | glutathione S-transferase | LigF |
| | | | [Rhodobacteraceae bacterium KLH11] | |
| | | | >gb\|EEE39034.1\|glutathione S- | |
| | | | transferase [Rhodobacteraceae | |
| | | | bacterium KLH11] | |
| 493 | 494 | ZP_05926645.1 | glutathione S-transferase [Vibrio sp. | LigF |
| | | | RC341] >gb\|EEX64947.1\|glutathione S- | |
| | | | transferase [Vibrio sp. RC341] | |
| 495 | 496 | ZP_06308936.1 | Glutathione S-transferase-like protein | LigF |
| | | | [Cylindrospermopsis raciborskii CS-505] | |
| | | | >gb\|EFA69058.1\|Glutathione S- | |
| | | | transferase-like protein | |
| | | | [Cylindrospermopsis raciborskii CS-505] | |
| 497 | 498 | ZP_06838829.1 | Glutathione S-transferase domain | LigF |
| | | | protein [Burkholderia sp. Ch1-1] | |
| | | | >gb\|EFG73275.1\|Glutathione S- | |
| | | | transferase domain protein [Burkholderia | |
| | | | sp. Ch1-1] | |
| 499 | 500 | ZP_08104209.1 | glutathione S-transferase III [Vibrio | LigF |
| | | | sinaloensis DSM 21326] | |
| | | | >gb\|EGA68654.1\|glutathione S- | |
| | | | transferase III [Vibrio sinaloensis DSM | |
| | | | 21326] | |
| 501 | 502 | ZP_08275708.1 | Glutathione S-transferase | LigF |
| | | | [Oxalobacteraceae bacterium | |
| | | | IMCC9480] >gb\|EGF30821.1\| | |
| | | | Glutathione S-transferase | |
| | | | [Oxalobacteraceae bacterium | |
| | | | IMCC9480] | |
| 503 | 504 | ZP_08409706.1 | glutathione S-transferase | LigF |
| | | | [Pseudoalteromonas haloplanktis | |
| | | | ANT/505] >gb\|EGI73123.1\|glutathione S [symbol] | |
| | | | transferase [Pseudoalteromonas | |
| | | | haloplanktis ANT/505] | |
| 505 | 506 | ZP_08565123.1 | glutathione S-transferase [Shewanella | LigF |
| | | | sp. HN-41] >gb\|EGM70872.1\| | |
| | | | glutathione S-transferase [Shewanella | |
| | | | sp. HN-41] | |
| 507 | 508 | CAA12269.1 | ORF 3 [Sphingomonas sp. RW5] | LigF |
| 509 | 510 | CAC94002.1 | glutathione transferase [Triticum | LigF |
| | | | aestivum] | |
| 511 | 512 | NP_967294.1 | maleylacetoacetate isomerase/ | LigF |
| | | | glutathione S-transferase [Bdellovibrio | |
| | | | bacteriovorus HD100] | |
| | | | >emb\|CAE77948.1\|maleylacetoacetate | |
| | | | isomerase/glutathione S-transferase | |
| | | | [Bdellovibrio bacteriovorus HD100] | |
| 513 | 514 | P30347.1 | RecName: Full = Protein ligF | LigF |
| | | | >dbj\|BAA02031.1\|beta-etherase | |
| | | | [Sphingomonas paucimobilis] | |
| | | | >prf\|\|1914145A beta etherase | |
| 515 | 516 | XP_002964271.1 | hypothetical protein | LigF |
| | | | SELMODRAFT_142654 [Selaginella | |
| | | | moellendorffii] >gb\|EFJ34604.1\| | |
| | | | hypothetical protein | |
| | | | SELMODRAFT_142654 [Selaginella | |
| | | | moellendorffii] | |
| 517 | 518 | YP_001021314.1 | glutathione S-transferase-like protein | LigF |
| | | | [Methylibium petroleiphilum PM1] | |
| | | | >gb\|ABM95079.1\|glutathione S- | |
| | | | transferase-like protein [Methylibium | |
| | | | petroleiphilum PM1] | |
| 519 | 520 | YP_001862387.1 | glutathione S-transferase domain- | LigF |
| | | | containing protein [Burkholderia | |
| | | | phymatum STM815] >gb\|ACC75341.1\| | |
| | | | Glutathione S-transferase domain | |
| | | | [Burkholderia phymatum STM815] | |
| 521 | 522 | YP_002130750.1 | glutathione S-transferase | LigF |
| | | | [Phenylobacterium zucineum HLK1] | |
| | | | >gb\|ACG78321.1\|glutathione S- | |
| | | | transferase [Phenylobacterium zucineum | |
| | | | HLK1] | |
| 523 | 524 | YP_002825255.1 | glutathione S-transferase [Sinorhizobium | LigF |
| | | | fredii NGR234] >gb\|ACP24502.1\| | |
| | | | glutathione S-transferase [Sinorhizobium | |
| | | | fredii NGR234] | |
| 525 | 526 | YP_003908670.1 | glutathione S-transferase domain- | LigF |
| | | | containing protein [Burkholderia sp. | |
| | | | CCGE1003] >gb\|ADN59379.1\| | |
| | | | Glutathione S-transferase domain | |
| | | | protein [Burkholderia sp. CCGE1003] | |
| 527 | 528 | YP_004154430.1 | glutathione s-transferase domain- | LigF |
| | | | containing protein [Variovorax paradoxus | |
| | | | EPS] >gb\|ADU36319.1\|Glutathione S- | |
| | | | transferase domain [Variovorax | |
| | | | paradoxus EPS] | |
| 529 | 530 | YP_004229981.1 | glutathione S-transferase domain- | LigF |
| | | | containing protein [Burkholderia sp. | |
| | | | CCGE1001] >gb\|ADX56921.1\| | |
| | | | Glutathione S-transferase domain | |
| | | | protein [Burkholderia sp. CCGE1001] | |
| 531 | 532 | YP_004302768.1 | glutathione S-transferase, N-terminal | LigF |
| | | | domain protein [Polymorphum gilvum | |
| | | | SL003B-26A1] >gb\|ADZ69468.1\| | |
| | | | Glutathione S-transferase, N-terminal | |
| | | | domain protein [Polymorphum gilvum | |
| | | | SL003B-26A1] | |
| 533 | 534 | YP_004533892.1 | glutathione S-transferase-like protein | LigF |
| | | | [Novosphingobium sp. PP1Y] | |
| | | | >emb\|CCA92074.1\|glutathione S- | |
| | | | transferase-like [Novosphingobium sp. | |
| | | | PP1Y] | |
| 535 | 536 | YP_004533893.1 | glutathione S-transferase-like protein | LigF |
| | | | [Novosphingobium sp. PP1Y] | |
| | | | >emb\|CCA92075.1\|glutathione S- | |
| | | | transferase-like [Novosphingobium sp. | |
| | | | PP1Y] | |
| 537 | 538 | YP_004533905.1 | glutathione S-transferase-like protein | LigF |
| | | | [Novosphingobium sp. PP1Y] | |
| | | | >emb\|CCA92087.1\|glutathione S- | |
| | | | transferase-like [Novosphingobium sp. | |
| | | | PP1Y] | |
| 539 | 540 | YP_497364.1 | glutathione S-transferase-like protein | LigF |
| | | | [Novosphingobium aromaticivorans DSM | |
| | | | 12444] >gb\|ABD26530.1\|glutathione S- | |
| | | | transferase-like protein | |
| | | | [Novosphingobium aromaticivorans DSM | |
| | | | 12444] | |
| 541 | 542 | YP_498135.1 | glutathione S-transferase-like protein | LigF |
| | | | [Novosphingobium aromaticivorans DSM | |
| | | | 12444] >gb\|ABD27301.1\|glutathione S- | |
| | | | transferase-like protein | |
| | | | [Novosphingobium aromaticivorans DSM | |
| | | | 12444] | |
| 543 | 544 | YP_498142.1 | glutathione S-transferase-like protein | LigF |
| | | | [Novosphingobium aromaticivorans DSM | |
| | | | 12444] >gb\|ABD27308.1\|glutathione S- | |
| | | | transferase-like protein | |
| | | | [Novosphingobium aromaticivorans DSM | |
| | | | 12444] | |
| 545 | 546 | YP_498143.1 | glutathione S-transferase-like protein | LigF |
| | | | [Novosphingobium aromaticivorans DSM | |
| | | | 12444] >gb\|ABD27309.1\|glutathione S- | |
| | | | transferase-like protein | |
| | | | [Novosphingobium aromaticivorans DSM | |
| | | | 12444] | |
| 547 | 548 | ZP_00952372.1 | maleylacetoacetate isomerase | LigF |
| | | | [Oceanicaulis alexandrii HTCC2633] | |
| | | | >gb\|EAP91525.1\|maleylacetoacetate | |
| | | | isomerase [Oceanicaulis alexandrii | |
| | | | HTCC2633] | |
| 549 | 550 | ZP_00959702.1 | glutathione S-transferase, putative | LigF |
| | | | [Roseovarius nubinhibens ISM] | |
| | | | >gb\|EAP78164.1\|glutathione S- | |
| | | | transferase, putative [Roseovarius | |
| | | | nubinhibens ISM] | |
| 551 | 552 | ZP_01034543.1 | glutathione S-transferase, putative | LigF |
| | | | [Roseovarius sp. 217] >gb\|EAQ27224.1\| | |
| | | | glutathione S-transferase, putative | |
| | | | [Roseovarius sp. 217] | |
| 553 | 554 | ZP_01057917.1 | glutathione S-transferase, putative | LigF |
| | | | [Roseobacter sp. MED193] | |
| | | | >gb\|EAQ44057.1\|glutathione S- | |
| | | | transferase, putative [Roseobacter sp. | |
| | | | MED193] | |
| 555 | 556 | ZP_01223510.1 | glutathione S-transferase [marine | LigF |
| | | | gamma proteobacterium HTCC2207] | |
| | | | >gb\|EAS48069.1\|glutathione S- | |
| | | | transferase [marine gamma | |
| | | | proteobacterium HTCC2207] | |
| 557 | 558 | ZP_01753989.1 | glutathione S-transferase, putative | LigF |
| | | | [Roseobacter sp. SK209-2-6] | |
| | | | >gb\|EBA17470.1\|glutathione S- | |
| | | | transferase, putative [Roseobacter sp. | |
| | | | SK209-2-6] | |
| 559 | 560 | ZP_02146800.1 | glutathione S-transferase-like protein | LigF |
| | | | [Phaeobacter gallaeciensis BS107] | |
| | | | >gb\|EDQ11817.1\|glutathione S- | |
| | | | transferase-like protein [Phaeobacter | |
| | | | gallaeciensis BS107] | |
| 561 | 562 | ZP_02150992.1 | glutathione S-transferase, putative | LigF |
| | | | [Phaeobacter gallaeciensis 2.10] | |
| | | | >gb\|EDQ07480.1\|glutathione S- | |
| | | | transferase, putative [Phaeobacter | |
| | | | gallaeciensis 2.10] | |
| 563 | 564 | ZP_05073592.1 | glutathione S-transferase 2 | LigF |
| | | | [Rhodobacterales bacterium HTCC2083] | |
| | | | >gb\|EDZ41252.1\|glutathione S- | |
| | | | transferase 2 [Rhodobacteraceae | |
| | | | bacterium HTCC2083] | |
| 565 | 566 | ZP_05077451.1 | glutathione S-transferase | LigF |
| | | | [Rhodobacterales bacterium Y4I] | |
| | | | >gb\|EDZ45430.1\|glutathione S- | |
| | | | transferase [Rhodobacterales bacterium | |
| | | | Y4I] | |
| 567 | 568 | ZP_05087035.1 | Glutathione S-transferase, N-terminal | LigF |
| | | | domain protein [Pseudovibrio sp. JE062] | |
| | | | >gb\|EEA92555.1\|Glutathione S- | |
| | | | transferase, N-terminal domain protein | |
| | | | [Pseudovibrio sp. JE062] | |
| 569 | 570 | ZP_05089424.1 | glutathione S-transferase [Ruegeria sp. | LigF |
| | | | R11] >gb\|EEB71116.1\|glutathione S- | |
| | | | transferase [Ruegeria sp. R11] | |
| 571 | 572 | ZP_05126316.1 | protein LigF [gamma proteobacterium | LigF |
| | | | NOR5-3] >gb\|EED32863.1\|protein LigF | |
| | | | [gamma proteobacterium NOR5-3] | |
| 573 | 574 | ZP_05126823.1 | maleylacetoacetate isomerase [gamma | LigF |
| | | | proteobacterium NOR5-3] | |
| | | | >gb\|EED33370.1\|maleylacetoacetate | |
| | | | isomerase [gamma proteobacterium | |
| | | | NOR5-3] | |
| 575 | 576 | ZP_05741946.1 | glutathione S-transferase [Silicibacter sp. | LigF |
| | | | TrichCH4B] >gb\|EEW58747.1\| | |
| | | | glutathione S-transferase [Silicibacter sp. | |
| | | | TrichCH4B] | |

[symbol]

indicates data missing or illegible when filed

TABLE 18

| PROTEIN | GENE | GENBANK | | |
|---|---|---|---|---|
| SEQ ID | SEQ ID | ACCESSION | | |
| NO: | NO: | NO: | DESCRIPTION: | TYPE |
| 577 | 578 | BAA77216.1 | glutathione S-transferase homolog | LigG |
| | | | [Sphingomonas paucimobilis] | |
| 579 | 580 | YP_004533907. [symbol] | glutathione S-transferase family protein | LigG |
| | | | [Novosphingobium sp. PP1Y] | |
| | | | >emb\|CCA92089.1\|glutathione S- | |
| | | | transferase family protein | |
| 581 | 582 | YP_314808.1 | glutathione S-transferase family protein | LigG |
| | | | [Thiobacillus denitrificans ATCC 25259] | |
| | | | >gb\|AAZ97003.1\|glutathione S- | |
| | | | transferase family protein [Thiobacillus | |
| 583 | 584 | YP_167289.1 | glutathione S-transferase family protein | LigG |
| | | | [Ruegeria pomeroyi DSS-3] | |
| | | | >gb\|AAV95330.1\|glutathione S- | |
| | | | transferase family protein [Ruegeria | |
| 585 | 586 | ZP_01011943.1 | glutathione S-transferase family protein | LigG |
| | | | [Maritimibacter alkaliphilus HTCC2654] | |
| | | | >gb\|EAQ14262.1\|glutathione S- | |
| | | | transferase family protein | |
| 587 | 588 | YP_002540613. [symbol] | glutathione S-transferase protein | LigG |
| | | | [Agrobacterium radiobacter K84] | |
| | | | >gb\|ACM29018.1\|glutathione S- | |
| 589 | 590 | CAJ81793.1 | Novel glutathione S-transferase omega | LigG |
| | | | protein [Xenopus (Silurana) tropicalis] | |
| 591 | 592 | NP_001005086. [symbol] | glutathione S-transferase omega 2 | LigG |
| | | | [Xenopus (Silurana) tropicalis] | |
| | | | >gb\|AAH77010.1\|MGC89704 protein | |
| 593 | 594 | XP_624501.1 | PREDICTED: glutathione S-transferase | LigG |
| | | | omega-1 [Apis mellifera] | |
| 595 | 596 | XP_002029736. [symbol] | GM24932 [Drosophila sechellia] | LigG |
| | | | >gb\|EDW40722.1\|GM24932 [Drosophila | |
| 597 | 598 | NP_001002621. [symbol] | hypothetical protein LOC436894 [Danio | LigG |
| | | | rerio] >gb\|AAH75965.1\|Zgc: 92254 [Danio | |
| 599 | 600 | XP_002431486. [symbol] | predicted protein [Pediculus humanus | LigG |
| | | | corporis] >gb\|EEB18748.1\|predicted | |
| | | | protein [Pediculus humanus corporis] | |
| 601 | 602 | ADD18952.1 | glutathione S-transferase [Glossina | LigG |
| | | | morsitans morsitans] | |
| 603 | 604 | XP_002093444. [symbol] | GE21298 [Drosophila yakuba] | LigG |
| | | | >gb\|EDW93156.1\|GE21298 [Drosophila | |
| 605 | 606 | XP_002068563. [symbol] | GK20540 [Drosophila willistoni] | LigG |
| | | | >gb\|EDW79549.1\|GK20540 [Drosophila | |
| 607 | 608 | NP_001165912. [symbol] | glutathione S-transferase O1 [Nasonia | LigG |
| 609 | 610 | CAM34501.1 | putative glutathione S-transferase | LigG |
| | | | [Cotesia congregata] | |
| 611 | 612 | XP_421747.1 | PREDICTED: similar to glutathione-S- | LigG |
| | | | transferase homolog isoform 2 [Gallus | |
| 613 | 614 | XP_002135069. [symbol] | GA23449 [Drosophila pseudoobscura | LigG |
| | | | pseudoobscura] >gb\|EDY73696.1\| | |
| | | | GA23449 [Drosophila pseudoobscura | |
| 615 | 616 | NP_034492.1 | glutathione S-transferase omega-1 [Mus | LigG |
| | | | musculus] | |
| | | | >sp\|O09131.2\|GSTO1_MOUSE | |
| | | | RecName: Full = Glutathione S-transferase | |
| | | | omega-1; Short = GSTO-1; AltName: | |
| | | | Full = p28 >gb\|AAB70110.1\|glutathione-S- | |
| | | | transferase homolog [Mus musculus] | |
| | | | >dbj\|BAC25667.1\|unnamed protein | |
| | | | product [Mus musculus] | |
| | | | >gb\|AAH85165.1\|Glutathione S- | |
| | | | transferase omega 1 [Mus musculus] | |
| | | | >dbj\|BAE27469.1\|unnamed protein | |
| | | | product [Mus musculus] | |
| | | | [symbol] | |
| 617 | 618 | ZP_03524422.1 | glutathione S-transferase domain- | LigG |
| | | | containing protein [Rhizobium etli GR56] | |
| 619 | 620 | NP_729388.1 | CG6673, isoform A [Drosophila | LigG |
| | | | melanogaster] >gb\|AAF50404.2\|CG6673, | |
| | | | isoform A [Drosophila melanogaster] | |
| | | | >gb\|ACZ02426.1\|glutathione S- | |
| 621 | 622 | ZP_08179398.1 | glutathione S-transferase [Xanthomonas | LigG |
| | | | vesicatoria ATCC 35937] | |
| | | | >gb\|EGD08414.1\|glutathione S- | |
| 623 | 624 | XP_003218563. [symbol] | PREDICTED: glutathione S-transferase | LigG |
| | | | omega-1-like isoform 1 [Anolis | |
| 625 | 626 | ABC86304.1 | IP16242p [Drosophila melanogaster] | LigG |
| 627 | 628 | XP_002026470. [symbol] | GL15567 [Drosophila persimilis] | LigG |
| | | | >gb\|EDW33419.1\|GL15567 [Drosophila | |
| 629 | 630 | NP_001108461. [symbol] | glutathione S-transferase omega 4 | LigG |
| | | | [Bombyx mori] >gb\|ABY66601.1\| | |
| | | | glutathione S-transferase 13 [Bombyx | |
| 631 | 632 | NP_999215.1 | glutathione S-transferase omega-1 [Sus | LigG |
| | | | scrofa] >ref\|XP_001929519.1\| | |
| | | | PREDICTED: glutathione S-transferase | |
| | | | omega-1-like [Sus scrofa] | |
| | | | >sp\|Q9N1F5.2\|GSTO1_PIG RecName: | |
| | | | Full = Glutathione S-transferase omega-1; | |
| | | | Short = GSTO-1; AltName: | |
| | | | Full = Glutathione-dependent | |
| | | | dehydroascorbate reductase | |
| 633 | 634 | NP_001007373. [symbol] | hypothetical protein LOC492500 [Danio | LigG |
| | | | rerio] >gb\|AAH85467.1\|Zgc: 101897 | |
| | | | [Danio rerio] >gb\|AAI65433.1\|Zgc: 101897 | |
| 635 | 636 | YP_001566654. [symbol] | glutathione S-transferase domain- | LigG |
| | | | containing protein [Delftia acidovorans | |
| | | | SPH-1] >gb\|ABX38269.1\|Glutathione S- | |
| | | | transferase domain [Delftia acidovorans | |
| 637 | 638 | ADY80021.1 | omega class glutathione S-transferase | LigG |
| | | | [Oplegnathus fasciatus] | |
| 639 | 640 | YP_001329158. [symbol] | glutathione S-transferase domain- | LigG |
| | | | containing protein [Sinorhizobium | |
| | | | medicae WSM419] >gb\|ABR62323.1\| | |
| | | | Glutathione S-transferase domain | |
| 641 | 642 | NP_001084924. [symbol] | hypothetical protein LOC431979 | LigG |
| | | | [Xenopus laevis] >gb\|AAH70673.1\| | |
| | | | MGC82327 protein [Xenopus laevis] | |
| 643 | 644 | XP_003396907. [symbol] | PREDICTED: glutathione S-transferase | LigG |
| | | | omega-1-like [Bombus terrestris] | |
| 645 | 646 | XP_001368758. [symbol] | PREDICTED: glutathione S-transferase | LigG |
| | | | omega-1-like isoform 1 [Monodelphis | |
| 647 | 648 | XP_001983981. [symbol] | GH16193 [Drosophila grimshawi] | LigG |
| | | | >gb\|EDV96329.1\|GH16193 [Drosophila | |
| 649 | 650 | ADK66966.1 | glutathione s-transferase [Chironomus | LigG |
| 651 | 652 | XP_001232808. [symbol] | PREDICTED: similar to glutathione-S- | LigG |
| | | | transferase homolog isoform 1 [Gallus | |
| 653 | 654 | XP_002068565. [symbol] | GK20354 [Drosophila willistoni] | LigG |
| | | | >gb\|EDW79551.1\|GK20354 [Drosophila | |
| 655 | 656 | YP_001611239. [symbol] | hypothetical protein sce0602 [Sorangium | LigG |
| | | | cellulosum ‘So ce 56’] >emb\|CAN90759.1\| | |
| | | | gst2 [Sorangium cellulosum ‘So ce 56’] | |
| 657 | 658 | XP_001499427. [symbol] | PREDICTED: glutathione S-transferase | LigG |
| | | | omega-1-like isoform 1 [Equus caballus] | |
| 659 | 660 | NP_384409.1 | putative glutathione S-transferase protein | LigG |
| | | | [Sinorhizobium meliloti 1021] | |
| | | | >ref\|YP_004550950.1\|glutathione S- | |
| | | | transferase domain-containing protein | |
| | | | [Sinorhizobium meliloti AK83] | |
| | | | >emb\|CAC41740.1\|Putative glutathione | |
| | | | S-transferase [Sinorhizobium meliloti | |
| | | | 1021] >gb\|AEG06303.1\|Glutathione S- | |
| | | | transferase domain protein | |
| | | | [Sinorhizobium meliloti BL225C] | |
| | | | >gb\|AEG55336.1\|Glutathione S- | |
| | | | [symbol] | |
| 661 | 662 | CAG05035.1 | unnamed protein product [Tetraodon | LigG |
| 663 | 664 | ZP_01365353.1 | hypothetical protein PaerPA_01002475 | LigG |
| | | | [Pseudomonas aeruginosa PACS2] | |
| | | | >ref\|YP_002440902.1\| | |
| | | | maleylacetoacetate isomerase | |
| | | | [Pseudomonas aeruginosa LESB58] | |
| | | | >ref\|ZP_04928412.1\|maleylacetoacetate | |
| | | | isomerase [Pseudomonas aeruginosa | |
| | | | C3719] >gb\|EAZ52531.1\| | |
| | | | maleylacetoacetate isomerase | |
| | | | [Pseudomonas aeruginosa C3719] | |
| | | | >emb\|CAW28043.1\|maleylacetoacetate | |
| | | | isomerase [Pseudomonas aeruginosa | |
| | | | [symbol] | |
| 665 | 666 | YP_001348642. [symbol] | maleylacetoacetate isomerase | LigG |
| | | | [Pseudomonas aeruginosa PA7] | |
| | | | >gb\|ABR84080.1\|maleylacetoacetate | |
| | | | isomerase [Pseudomonas aeruginosa | |
| 667 | 668 | ZP_04933765.1 | maleylacetoacetate isomerase | LigG |
| | | | [Pseudomonas aeruginosa 2192] | |
| | | | >gb\|EAZ57884.1\|maleylacetoacetate | |
| | | | isomerase [Pseudomonas aeruginosa | |
| 669 | 670 | NP_250697.1 | maleylacetoacetate isomerase | LigG |
| | | | [Pseudomonas aeruginosa PAO1] | |
| | | | >sp\|P57109.1\|MAAI_PSEAE RecName: | |
| | | | Full = Maleylacetoacetate isomerase; | |
| | | | Short = MAAI | |
| | | | >gb\|AAG05395.1\|AE004627_3 | |
| 671 | 672 | EFN59352.1 | hypothetical protein | LigG |
| | | | CHLNCDRAFT_137800 [Chlorella | |
| 673 | 674 | YP_002945584. [symbol] | glutathione S-transferase domain- | LigG |
| | | | containing protein [Variovorax paradoxus | |
| | | | S110] >gb\|ACS20318.1\|Glutathione S- | |
| | | | transferase domain protein [Variovorax | |
| 675 | 676 | XP_002197460. [symbol] | PREDICTED: glutathione S-transferase | LigG |
| | | | omega 1 [Taeniopygia guttata] | |
| 677 | 678 | XP_001971643. [symbol] | GG15075 [Drosophila erecta] | LigG |
| | | | >gb\|EDV50669.1\|GG15075 [Drosophila | |
| 679 | 680 | NP_001155757. [symbol] | glutathione S-transferase omega-1-like | LigG |
| | | | [Acyrthosiphon pisum] >dbj\|BAH71013.1\| | |
| | | | ACYPI008340 [Acyrthosiphon pisum] | |
| 681 | 682 | XP_002026468. [symbol] | GL15565 [Drosophila persimilis] | LigG |
| | | | >gb\|EDW33417.1\|GL15565 [Drosophila | |
| 683 | 684 | XP_001353820. [symbol] | GA19760 [Drosophila pseudoobscura | LigG |
| | | | pseudoobscura] >gb\|EAL29555.1\| | |
| | | | GA19760 [Drosophila pseudoobscura | |
| 685 | 686 | YP_791232.1 | maleylacetoacetate isomerase | LigG |
| | | | [Pseudomonas aeruginosa UCBPP-PA14] | |
| | | | >gb\|ABJ11194.1\|maleylacetoacetate | |
| | | | isomerase [Pseudomonas aeruginosa | |
| 687 | 688 | ZP_06879058.1 | maleylacetoacetate isomerase | LigG |
| | | | [Pseudomonas aeruginosa PAb1] | |
| | | | >ref\|ZP_07797003.1\|maleylacetoacetate | |
| | | | isomerase [Pseudomonas aeruginosa | |
| | | | 39016] >gb\|EFQ42099.1\| | |
| | | | maleylacetoacetate isomerase | |
| | | | [Pseudomonas aeruginosa 39016] | |
| | | | >gb\|EGM14661.1\|maleylacetoacetate | |
| 689 | 690 | EFZ22366.1 | hypothetical protein SINV_14968 | LigG |
| 691 | 692 | ZP_03527925.1 | Glutathione S-transferase domain | LigG |
| | | | [Rhizobium etli CIAT 894] | |
| 693 | 694 | ABD77536.1 | hypothetical protein [Ictalurus punctatus] | LigG |
| 695 | 696 | XP_002756473. [symbol] | PREDICTED: glutathione S-transferase | LigG |
| | | | omega-1-like [Callithrix jacchus] | |
| 697 | 698 | XP_001636996. [symbol] | predicted protein [Nematostella vectensis] | LigG |
| | | | >gb\|EDO44933.1\|predicted protein | |
| | | | [Nematostella vectensis] | |
| 699 | 700 | YP_467831.1 | glutathione S-transferase [Rhizobium etli | LigG |
| | | | CFN 42] >gb\|ABC89104.1\|glutathione S- | |
| | | | transferase protein [Rhizobium etli CFN | |
| 701 | 702 | NP_103005.1 | glutathione-S-transferase [Mesorhizobium | LigG |
| | | | loti MAFF303099] >dbj\|BAB48791.1\| | |
| | | | glutathione-S-transferase [Mesorhizobium | |
| 703 | 704 | ADY47623.1 | Glutathione transferase omega-1 [Ascaris | LigG |
| 705 | 706 | BAG36430.1 | unnamed protein product [Homo sapiens] | LigG |
| 707 | 708 | XP_002718774. [symbol] | PREDICTED: glutathione-S-transferase | LigG |
| | | | omega 1 [Oryctolagus cuniculus] | |
| 709 | 710 | 3LFL_A | Chain A, Crystal Structure Of Human | LigG |
| | | | Glutathione Transferase Omega 1, Delta | |
| | | | 155 >pdb\|3LFL\|B Chain B, Crystal | |
| | | | Structure Of Human Glutathione | |
| | | | Transferase Omega 1, Delta 155 | |
| | | | >pdb\|3LFL\|C Chain C, Crystal Structure | |
| 711 | 712 | XP_002805857. [symbol] | PREDICTED: glutathione S-transferase | LigG |
| | | | omega-1-like [Macaca mulatta] | |
| | | | >gb\|ABO21635.1\|glutathione S- | |
| 713 | 714 | NP_001007603. [symbol] | glutathione S-transferase omega-1 | LigG |
| | | | [Rattus norvegicus] >gb\|AAH79363.1\| | |
| | | | Glutathione S-transferase omega 1 | |
| | | | [Rattus norvegicus] >gb\|EDL94393.1\| | |
| | | | glutathione S-transferase omega 1, | |
| 715 | 716 | XP_535007.1 | PREDICTED: similar to glutathione-S- | LigG |
| | | | transferase omega 1 isoform 1 [Canis | |
| 717 | 718 | NP_004823.1 | glutathione S-transferase omega-1 | LigG |
| | | | isoform 1 [Homo sapiens] | |
| | | | >sp\|P78417.2\|GSTO1_HUMAN | |
| | | | RecName: Full = Glutathione S-transferase | |
| | | | omega-1; Short = GSTO-1 >pdb\|1EEM\|A | |
| | | | Chain A, Glutathione Transferase From | |
| | | | Homo sapiens | |
| | | | >gb\|AAF73376.1\|AF212303_1 glutathione | |
| | | | transferase omega [Homo sapiens] | |
| | | | >gb\|AAB70109.1\|glutathione-S- | |
| | | | transferase homolog [Homo sapiens] | |
| | | | >gb\|AAH00127.1\|Glutathione S- | |
| | | | transferase omega 1 [Homo sapiens] | |
| | | | >gb\|AAV68046.1\|glutathione S- | |
| | | | transferase omega 1-1 [Homo sapiens] | |
| | | | [symbol] | |
| 719 | 720 | XP_002758417. [symbol] | PREDICTED: glutathione S-transferase | LigG |
| | | | omega-1-like [Callithrix jacchus] | |
| 721 | 722 | XP_003218564. [symbol] | PREDICTED: glutathione S-transferase | LigG |
| | | | omega-1-like isoform 2 [Anolis | |
| 723 | 724 | EFN62827.1 | Glutathione transferase omega-1 | LigG |
| | | | [Camponotus floridanus] | |
| 725 | 726 | XP_508020.3 | PREDICTED: glutathione S-transferase | LigG |
| | | | omega-1 isoform 3 [Pan troglodytes] | |
| 727 | 728 | CAD97673.1 | hypothetical protein [Homo sapiens] | LigG |
| 729 | 730 | BAJ20927.1 | glutathione S-transferase omega 1 | LigG |
| | | | [synthetic construct] | |
| 731 | 732 | ACR43779.1 | glutathione S-transferase [Chironomus | LigG |
| 733 | 734 | Q9Z339.2 | RecName: Full = Glutathione S-transferase | LigG |
| | | | omega-1; Short = GSTO-1; AltName: | |
| | | | Full = Glutathione-dependent | |
| | | | dehydroascorbate reductase | |
| | | | >gb\|ACI32122.1\|glutathione S- | |
| 735 | 736 | XP_001956909. [symbol] | GF10159 [Drosophila ananassae] | LigG |
| | | | >gb\|EDV39715.1\|GF10159 [Drosophila | |
| 737 | 738 | XP_001742278. [symbol] | hypothetical protein [Monosiga brevicollis | LigG |
| | | | MX1] >gb\|EDQ92516.1\|predicted protein | |
| | | | [Monosiga brevicollis MX1] | |
| 739 | 740 | XP_002821176. [symbol] | PREDICTED: glutathione S-transferase | LigG |
| | | | omega-1-like [Pongo abelii] | |
| 741 | 742 | XP_003255483. [symbol] | PREDICTED: glutathione S-transferase | LigG |
| | | | omega-1-like isoform 1 [Nomascus | |
| 743 | 744 | YP_325490.1 | glutathione S-transferase-like protein | LigG |
| | | | [Anabaena variabilis ATCC 29413] | |
| | | | >gb\|ABA24595.1\|Glutathione S- | |
| | | | transferase-like protein [Anabaena | |
| 745 | 746 | XP_003208190. [symbol] | PREDICTED: glutathione S-transferase | LigG |
| | | | omega-1-like [Meleagris gallopavo] | |
| 747 | 748 | XP_002068562. [symbol] | GK20539 [Drosophila willistoni] | LigG |
| | | | >gb\|EDW79548.1\|GK20539 [Drosophila | |
| 749 | 750 | XP_001956911. [symbol] | GF10161 [Drosophila ananassae] | LigG |
| | | | >gb\|EDV39717.1\|GF10161 [Drosophila | |
| 751 | 752 | ABV24048.1 | gluthathione S-transferase omega | LigG |
| | | | [Takifugu obscurus] | |
| 753 | 754 | ZP_05086262.1 | putative glutathione S-transferase protein | LigG |
| | | | [Pseudovibrio sp. JE062] | |
| | | | >gb\|EEA93528.1\|putative glutathione S- | |
| | | | transferase protein [Pseudovibrio sp. | |
| 755 | 756 | AAI28951.1 | LOC100037104 protein [Xenopus laevis] | LigG |
| 757 | 758 | XP_001956910. [symbol] | GF10160 [Drosophila ananassae] | LigG |
| | | | >gb\|EDV39716.1\|GF10160 [Drosophila | |
| 759 | 760 | NP_001099052. [symbol] | glutathione S-transferase omega 2 | LigG |
| | | | [Xenopus laevis] >gb\|AAI53758.1\| | |
| | | | LOC100037104 protein [Xenopus laevis] | |
| 761 | 762 | ZP_03503214.1 | Glutathione S-transferase domain | LigG |
| | | | [Rhizobium etli Kim 5] | |
| 763 | 764 | XP_002046961. [symbol] | GJ12198 [Drosophila virilis] | LigG |
| | | | >gb\|EDW69303.1\|GJ12198 [Drosophila | |
| 765 | 766 | XP_001956912. [symbol] | GF24331 [Drosophila ananassae] | LigG |
| | | | >gb\|EDV39718.1\|GF24331 [Drosophila | |
| 767 | 768 | XP_001368790. [symbol] | PREDICTED: glutathione S-transferase | LigG |
| | | | omega-1-like isoform 1 [Monodelphis | |
| 769 | 770 | ZP_06308936.1 | Glutathione S-transferase-like protein | LigG |
| | | | [Cylindrospermopsis raciborskii CS-505] | |
| | | | >gb\|EFA69058.1\|Glutathione S- | |
| | | | transferase-like protein | |
| 771 | 772 | ABJ15788.1 | glutathione S-transferase omega 1 | LigG |
| | | | [Bombyx mandarina] >dbj\|BAF91356.1\| | |
| | | | omega-class glutathione S-transferase | |
| 773 | 774 | NP_001037406. [symbol] | glutathione S-transferase omega 2 | LigG |
| | | | [Bombyx mori] >gb\|ABC79689.1\| | |
| | | | glutathione S-transferase 6 [Bombyx mori] | |
| 775 | 776 | NP_001040131. [symbol] | glutathione S-transferase omega 1 | LigG |
| | | | [Bombyx mori] >gb\|ABD36128.1\| | |
| | | | glutathione S-transferase omega 1 | |

[symbol]

indicates data missing or illegible when filed

TABLE 19

| PROTEIN | GENE | | | |
|---|---|---|---|---|
| SEQ ID | SEQ ID | GENBANK | | |
| NO: | NO: | ACCESSION NO: | DESCRIPTION: | TYPE |
| 777 | 778 | Q01198.1 | RecName: Full = C alpha-dehydrogenase | LigD |
| | | | >dbj\|BAA02030.1\|C alpha-dehydrogenase | |
| | | | [Sphingomonas paucimobilis] | |
| | | | >dbj\|BAA01953.1\|C alpha-dehydrogenase | |
| | | | [Sphingomonas paucimobilis] | |
| | | | >gb\|AAC60455.1\|C alpha-dehydrogenase | |
| | | | [Sphingomonas paucimobilis] | |
| 779 | 780 | YP_495487.1 | short-chain dehydrogenase/reductase SDR | LigD |
| | | | [Novosphingobium aromaticivorans DSM | |
| | | | 12444] >gb\|ABD24653.1\|short-chain | |
| | | | dehydrogenase/reductase SDR | |
| | | | [Novosphingobium aromaticivorans DSM | |
| | | | 12444] | |
| 781 | 782 | YP_004533898.1 | short-chain dehydrogenase/reductase SDR | LigD |
| | | | [Novosphingobium sp. PP1Y] | |
| | | | >emb\|CCA92080.1\|short-chain | |
| | | | dehydrogenase/reductase SDR | |
| | | | [Novosphingobium sp. PP1Y] | |
| 783 | 784 | BAH56687.1 | Calpha-dehydrogenase [Sphingobium sp. | LigD |
| | | | SYK-6] | |
| 785 | 786 | YP_004533921.1 | short-chain dehydrogenase/reductase SDR | LigD |
| | | | [Novosphingobium sp. PP1Y] | |
| | | | >emb\|CCA92103.1\|short-chain | |
| | | | dehydrogenase/reductase SDR | |
| | | | [Novosphingobium sp. PP1Y] | |
| 787 | 788 | YP_496072.1 | short-chain dehydrogenase/reductase SDR | LigD |
| | | | [Novosphingobium aromaticivorans DSM | |
| | | | 12444] >gb\|ABD25238.1\|short-chain | |
| | | | dehydrogenase/reductase SDR | |
| | | | [Novosphingobium aromaticivorans DSM | |
| | | | 12444] | |
| 789 | 790 | 3IOY_A | Chain A, Structure Of Putative Short-Chain | LigD |
| | | | Dehydrogenase (Saro_0793) From | |
| | | | Novosphingobium Aromaticivorans | |
| | | | >pdb\|3IOY\|B Chain B, Structure Of Putative | |
| | | | Short-Chain Dehydrogenase (Saro_0793) | |
| | | | From Novosphingobium Aromaticivorans | |
| 791 | 792 | YP_496073.1 | short-chain dehydrogenase/reductase SDR | LigD |
| | | | [Novosphingobium aromaticivorans DSM | |
| | | | 12444] >gb\|ABD25239.1\|short-chain | |
| | | | dehydrogenase/reductase SDR | |
| | | | [Novosphingobium aromaticivorans DSM | |
| | | | 12444] | |
| 793 | 794 | BAH56683.1 | Calpha-dehydrogenase [Sphingobium sp. | LigD |
| | | | SYK-6] | |
| 795 | 796 | YP_004533920.1 | short-chain dehydrogenase/reductase SDR | LigD |
| | | | [Novosphingobium sp. PP1Y] | |
| | | | >emb\|CCA92102.1\|short-chain | |
| | | | dehydrogenase/reductase SDR | |
| | | | [Novosphingobium sp. PP1Y] | |
| 797 | 798 | YP_003592832.1 | short-chain dehydrogenase/reductase SDR | LigD |
| | | | [Caulobacter segnis ATCC 21756] | |
| | | | >gb\|ADG10214.1\|short-chain | |
| | | | dehydrogenase/reductase SDR [Caulobacter | |
| | | | segnis ATCC 21756] | |
| 799 | 800 | YP_495984.1 | short-chain dehydrogenase/reductase SDR | LigD |
| | | | [Novosphingobium aromaticivorans DSM | |
| | | | 12444] >gb\|ABD25150.1\|short-chain | |
| | | | dehydrogenase/reductase SDR | |
| | | | [Novosphingobium aromaticivorans DSM | |
| | | | 12444] | |
| 801 | 802 | YP_497149.1 | short-chain dehydrogenase/reductase SDR | LigD |
| | | | [Novosphingobium aromaticivorans DSM | |
| | | | 12444] >gb\|ABD26315.1\|short-chain | |
| | | | dehydrogenase/reductase SDR | |
| | | | [Novosphingobium aromaticivorans DSM | |
| | | | 12444] | |
| 803 | 804 | YP_003592830.1 | short-chain dehydrogenase/reductase SDR | LigD |
| | | | [Caulobacter segnis ATCC 21756] | |
| | | | >gb\|ADG10212.1\|short-chain | |
| | | | dehydrogenase/reductase SDR [Caulobacter | |
| | | | segnis ATCC 21756] | |
| 805 | 806 | YP_001260886.1 | short-chain dehydrogenase/reductase SDR | LigD |
| | | | [Sphingomonas wittichii RW1] | |
| | | | >gb\|ABQ66748.1\|short-chain | |
| | | | dehydrogenase/reductase SDR | |
| | | | [Sphingomonas wittichii RW1] | |
| 807 | 808 | YP_001413979.1 | short-chain dehydrogenase/reductase SDR | LigD |
| | | | [Parvibaculum lavamentivorans DS-1] | |
| | | | >gb\|ABS64322.1\|short-chain | |
| | | | dehydrogenase/reductase SDR | |
| | | | [Parvibaculum lavamentivorans DS-1] | |
| 809 | 810 | YP_001412300.1 | short-chain dehydrogenase/reductase SDR | LigD |
| | | | [Parvibaculum lavamentivorans DS-1] | |
| | | | >gb\|ABS62643.1\|short-chain | |
| | | | dehydrogenase/reductase SDR | |
| | | | [Parvibaculum lavamentivorans DS-1] | |
| 811 | 812 | YP_001412299.1 | short-chain dehydrogenase/reductase SDR | LigD |
| | | | [Parvibaculum lavamentivorans DS-1] | |
| | | | >gb\|ABS62642.1\|short-chain | |
| | | | dehydrogenase/reductase SDR | |
| | | | [Parvibaculum lavamentivorans DS-1] | |
| 813 | 814 | BAH56685.1 | Calpha-dehydrogenase [Sphingobium sp. | LigD |
| | | | SYK-6] | |
| 815 | 816 | NP_959644.1 | short chain dehydrogenase [Mycobacterium | LigD |
| | | | avium subsp. paratuberculosis K-10] | |
| | | | >ref\|YP_880159.1\|short chain | |
| | | | dehydrogenase [Mycobacterium avium 104] | |
| | | | >ref\|ZP_05215302.1\|short chain | |
| | | | dehydrogenase [Mycobacterium avium | |
| | | | subsp. avium ATCC 25291] | |
| | | | >gb\|AAS03027.1\|hypothetical protein | |
| | | | MAP_0710c [Mycobacterium avium subsp. | |
| | | | paratuberculosis K-10] >gb\|ABK67661.1\| | |
| | | | short chain dehydrogenase [Mycobacterium | |
| | | | avium 104] >gb\|EGO40035.1\|short-chain | |
| | | | alcohol dehydrogenase [Mycobacterium | |
| | | | avium subsp. paratuberculosis S397] | |
| 817 | 818 | ZP_08717023.1 | short chain dehydrogenase [Mycobacterium | LigD |
| | | | colombiense CECT 3035] >gb\|EGT85268.1\| | |
| | | | short chain dehydrogenase [Mycobacterium | |
| | | | colombiense CECT 3035] | |
| 819 | 820 | ZP_05127447.1 | oxidoreductase, short chain | LigD |
| | | | dehydrogenase/reductase family protein | |
| | | | [gamma proteobacterium NOR5-3] | |
| | | | >gb\|EED33994.1\|oxidoreductase, short | |
| | | | chain dehydrogenase/reductase family | |
| | | | protein [gamma proteobacterium NOR5-3] | |
| 821 | 822 | YP_004555419.1 | Estradiol 17-beta-dehydrogenase | LigD |
| | | | [Sphingobium chlorophenolicum L-1] | |
| | | | >gb\|AEG50913.1\|Estradiol 17-beta- | |
| | | | dehydrogenase [Sphingobium | |
| | | | chlorophenolicum L-1] | |
| 823 | 824 | YP_004230838.1 | short-chain dehydrogenase/reductase SDR | LigD |
| | | | [Burkholderia sp. CCGE1001] | |
| | | | >gb\|ADX57778.1\|short-chain | |
| | | | dehydrogenase/reductase SDR | |
| | | | [Burkholderia sp. CCGE1001] | |
| 825 | 826 | YP_004284589.1 | putative oxidoreductase [Acidiphilium | LigD |
| | | | multivorum AIU301] >dbj\|BAJ81707.1\| | |
| | | | putative oxidoreductase [Acidiphilium | |
| | | | multivorum AIU301] | |
| 827 | 828 | YP_001235233.1 | hypothetical protein Acry_2115 [Acidiphilium | LigD |
| | | | cryptum JF-5] >gb\|ABQ31314.1\|short-chain | |
| | | | dehydrogenase/reductase SDR [Acidiphilium | |
| | | | cryptum JF-5] | |
| 829 | 830 | ZP_01617820.1 | hypothetical protein GP2143_09415 [marine | LigD |
| | | | gamma proteobacterium HTCC2143] | |
| | | | >gb\|EAW30413.1\|hypothetical protein | |
| | | | GP2143_09415 [marine gamma | |
| | | | proteobacterium HTCC2143] | |
| 831 | 832 | ZP_08629833.1 | short-chain dehydrogenase/reductase | LigD |
| | | | [Bradyrhizobiaceae bacterium SG-6C] | |
| | | | >gb\|EGP07476.1\|short-chain | |
| | | | dehydrogenase/reductase | |
| | | | [Bradyrhizobiaceae bacterium SG-6C] | |
| 833 | 834 | YP_001853014.1 | short-chain type dehydrogenase/reductase | LigD |
| | | | [Mycobacterium marinum M] | |
| | | | >gb\|ACC43159.1\|short-chain type | |
| | | | dehydrogenase/reductase [Mycobacterium | |
| | | | marinum M] | |
| 835 | 836 | YP_004754457.1 | short-chain dehydrogenase/reductase SDR | LigD |
| | | | [Collimonas fungivorans Ter331] | |
| | | | >gb\|AEK63634.1\|short-chain | |
| | | | dehydrogenase/reductase SDR [Collimonas | |
| | | | fungivorans Ter331] | |
| 837 | 838 | ZP_05129129.1 | short-chain dehydrogenase/reductase SDR | LigD |
| | | | [gamma proteobacterium NOR5-3] | |
| | | | >gb\|EED30944.1\|short-chain | |
| | | | dehydrogenase/reductase SDR [gamma | |
| | | | proteobacterium NOR5-3] | |
| 839 | 840 | ZP_05223648.1 | short chain dehydrogenase [Mycobacterium | LigD |
| | | | intracellulare ATCC 13950] | |
| 841 | 842 | YP_004555383.1 | short-chain dehydrogenase/reductase SDR | LigD |
| | | | [Sphingobium chlorophenolicum L-1] | |
| | | | >gb\|AEG50877.1\|short-chain | |
| | | | dehydrogenase/reductase SDR | |
| | | | [Sphingobium chlorophenolicum L-1] | |
| 843 | 844 | YP_976997.1 | short chain dehydrogenase [Mycobacterium | LigD |
| | | | bovis BCG str. Pasteur 1173P2] | |
| | | | >ref\|YP_002643932.1\|short-chain | |
| | | | dehydrogenase [Mycobacterium bovis BCG | |
| | | | str. Tokyo 172] >ref\|ZP_06432004.1\|short- | |
| | | | chain type dehydrogenase/reductase | |
| | | | [Mycobacterium tuberculosis T46] | |
| | | | >ref\|ZP_06449040.1\|short-chain type | |
| | | | dehydrogenase/reductase [Mycobacterium | |
| | | | tuberculosis T17] >ref\|ZP_06453700.1\|short [symbol] | |
| | | | chain type dehydrogenase/reductase | |
| | | | [Mycobacterium tuberculosis K85] | |
| | | | >ref\|ZP_06508748.1\|short-chain type | |
| | | | dehydrogenase/reductase [Mycobacterium | |
| | | | tuberculosis T92] >ref\|ZP_06512283.1\|short | |
| | | | chain dehydrogenase [Mycobacterium | |
| | | | tuberculosis EAS054] >ref\|YP_004722558.1\| | |
| | | | short-chain type dehydrogenase/reductase | |
| | | | [Mycobacterium africanum GM041182] | |
| | | | >emb\|CAL70889.1\|Putative short-chain type | |
| | | | dehydrogenase/reductase [Mycobacterium | |
| | | | bovis BCG str. Pasteur 1173P2] | |
| | | | >dbj\|BAH25164.1\|short-chain | |
| | | | dehydrogenase [Mycobacterium bovis BCG | |
| | | | str. Tokyo 172] >gb\|EFD12419.1\|short- | |
| | | | chain type dehydrogenase/reductase | |
| | | | [Mycobacterium tuberculosis T46] | |
| | | | >gb\|EFD42482.1\|short-chain type | |
| | | | dehydrogenase/reductase [Mycobacterium | |
| | | | tuberculosis K85] >gb\|EFD46215.1\|short- | |
| 845 | 846 | ZP_01101659.1 | Short-chain dehydrogenase/reductase SDR | LigD |
| | | | [Congregibacter litoralis KT71] | |
| | | | >gb\|EAQ98875.1\|Short-chain | |
| | | | dehydrogenase/reductase SDR | |
| | | | [Congregibacter litoralis KT71] | |
| 847 | 848 | ZP_01615364.1 | short chain dehydrogenase [marine gamma | LigD |
| | | | proteobacterium HTCC2143] | |
| | | | >gb\|EAW32447.1\|short chain | |
| | | | dehydrogenase [marine gamma | |
| | | | proteobacterium HTCC2143] | |
| 849 | 850 | ZP_06436160.1 | short-chain type dehydrogenase/reductase | LigD |
| | | | [Mycobacterium tuberculosis CPHL_A] | |
| | | | >gb\|EFD16575.1\|short-chain type | |
| | | | dehydrogenase/reductase [Mycobacterium | |
| | | | tuberculosis CPHL_A] | |
| 851 | 852 | NP_854532.1 | short chain dehydrogenase [Mycobacterium | LigD |
| | | | bovis AF2122/97] >emb\|CAD93736.1\| | |
| | | | PUTATIVE SHORT-CHAIN TYPE | |
| | | | DEHYDROGENASE/REDUCTASE | |
| | | | [Mycobacterium bovis AF2122/97] | |
| 853 | 854 | YP_004744317.1 | putative short-chain type | LigD |
| | | | dehydrogenase/reductase [Mycobacterium | |
| | | | canettii CIPT 140010059] | |
| | | | >emb\|CCC43191.1\|putative short-chain | |
| | | | type dehydrogenase/reductase | |
| | | | [Mycobacterium canettii CIPT 140010059] | |
| 855 | 856 | YP_003947586.1 | short-chain dehydrogenase/reductase sdr | LigD |
| | | | [Paenibacillus polymyxa SC2] | |
| | | | >gb\|ADO57345.1\|Short-chain | |
| | | | dehydrogenase/reductase SDR | |
| | | | [Paenibacillus polymyxa SC2] | |
| 857 | 858 | YP_003951191.1 | short-chain dehydrogenase/reductase | LigD |
| | | | [Stigmatella aurantiaca DW4/3-1] | |
| | | | >gb\|ADO69364.1\|Short-chain | |
| | | | dehydrogenase/reductase SDR [Stigmatella | |
| | | | aurantiaca DW4/3-1] | |
| 859 | 860 | YP_583994.1 | hypothetical protein Rmet_1846 | LigD |
| | | | [Cupriavidus metallidurans CH34] | |
| | | | >gb\|ABF08725.1\|conserved hypothetical | |
| | | | protein [Cupriavidus metallidurans CH34] | |
| 861 | 862 | NP_215366.1 | short chain dehydrogenase [Mycobacterium | LigD |
| | | | tuberculosis H37Rv] >ref\|YP_001282151.1\| | |
| | | | short chain dehydrogenase [Mycobacterium | |
| | | | tuberculosis H37Ra] >ref\|YP_001286813.1\| | |
| | | | short chain dehydrogenase [Mycobacterium | |
| | | | tuberculosis F11] >ref\|ZP_02549252.1\|short | |
| | | | chain dehydrogenase [Mycobacterium | |
| | | | tuberculosis H37Ra] >ref\|YP_003033128.1\| | |
| | | | short-chain type dehydrogenase/reductase | |
| | | | [Mycobacterium tuberculosis KZN 1435] | |
| | | | >ref\|ZP_04924487.1\|hypothetical protein | |
| | | | TBCG_00842 [Mycobacterium tuberculosis | |
| | | | C] >ref\|ZP_04979832.1\|hypothetical short- | |
| | | | chain type dehydrogenase/reductase | |
| | | | [Mycobacterium tuberculosis str. Haarlem] | |
| | | | >ref\|ZP_05140274.1\|short chain | |
| | | | dehydrogenase [Mycobacterium tuberculosis | |
| | | | ‘98-R604 INH-RIF-EM’] | |
| | | | >ref\|ZP_06444578.1\|short-chain type | |
| | | | dehydrogenase/reductase [Mycobacterium | |
| | | | tuberculosis KZN 605] >ref\|ZP_06503955.1\| | |
| | | | short chain dehydrogenase [Mycobacterium | |
| | | | tuberculosis 02_1987] >ref\|ZP_06516315.1\| | |
| | | | short chain dehydrogenase [Mycobacterium | |
| | | | tuberculosis T85] >ref\|ZP_06520361.1\|short [symbol] | |
| | | | chain type dehydrogenase/reductase | |
| | | | [Mycobacterium tuberculosis GM 1503] | |
| | | | >ref\|ZP_06802023.1\|short chain | |
| | | | dehydrogenase [Mycobacterium tuberculosis | |
| | | | 210] >ref\|ZP_06951148.1\|short chain | |
| 863 | 864 | YP_904525.1 | short chain dehydrogenase [Mycobacterium | LigD |
| | | | ulcerans Agy99] >gb\|ABL03054.1\|short- | |
| | | | chain type dehydrogenase/reductase | |
| | | | [Mycobacterium ulcerans Agy99] | |
| 865 | 866 | ZP_06851131.1 | short-chain dehydrogenase/reductase family | LigD |
| | | | oxidoreductase [Mycobacterium | |
| | | | parascrofulaceum ATCC BAA-614] | |
| | | | >gb\|EFG75472.1\|short-chain | |
| | | | dehydrogenase/reductase family | |
| | | | oxidoreductase [Mycobacterium | |
| | | | parascrofulaceum ATCC BAA-614] | |
| 867 | 868 | YP_003871369.1 | 3-oxoacyl-[acyl-carrier-protein] reductase (3- | LigD |
| | | | ketoacyl-acyl carrier protein reductase) | |
| | | | [Paenibacillus polymyxa E681] | |
| | | | >gb\|ADM70831.1\|3-oxoacyl-[acyl-carrier- | |
| | | | protein] reductase (3-ketoacyl-acyl carrier | |
| | | | protein reductase) [Paenibacillus polymyxa | |
| | | | E681] | |
| 869 | 870 | ZP_05094873.1 | oxidoreductase, short chain | LigD |
| | | | dehydrogenase/reductase family [marine | |
| | | | gamma proteobacterium HTCC2148] | |
| | | | >gb\|EEB78920.1\|oxidoreductase, short | |
| | | | chain dehydrogenase/reductase family | |
| | | | [marine gamma proteobacterium | |
| | | | HTCC2148] | |
| 871 | 872 | ZP_01224235.1 | probable oxidoreductase dehydrogenase | LigD |
| | | | signal peptide protein [marine gamma | |
| | | | proteobacterium HTCC2207] | |
| | | | >gb\|EAS47242.1\|probable oxidoreductase | |
| | | | dehydrogenase signal peptide protein | |
| | | | [marine gamma proteobacterium | |
| | | | HTCC2207] | |
| 873 | 874 | YP_634033.1 | short chain dehydrogenase [Myxococcus | LigD |
| | | | xanthus DK 1622] >gb\|ABF86178.1\| | |
| | | | oxidoreductase, short chain | |
| | | | dehydrogenase/reductase family | |
| | | | [Myxococcus xanthus DK 1622] | |
| 875 | 876 | ABL97174.1 | short-chain dehydrogenase/reductase | LigD |
| | | | [uncultured marine bacterium EB0_49D07] | |
| 877 | 878 | NP_335301.1 | short chain dehydrogenase [Mycobacterium | LigD |
| | | | tuberculosis CDC1551] | |
| | | | >ref\|ZP_07413312.2\|short-chain type | |
| | | | dehydrogenase/reductase [Mycobacterium | |
| | | | tuberculosis SUMu001] | |
| | | | >ref\|ZP_07668817.1\|short-chain type | |
| | | | dehydrogenase/reductase [Mycobacterium | |
| | | | tuberculosis SUMu010] | |
| | | | >ref\|ZP_07669069.1\|short-chain type | |
| | | | dehydrogenase/reductase [Mycobacterium | |
| | | | tuberculosis SUMu011] >gb\|AAK45115.1\| | |
| | | | oxidoreductase, short-chain | |
| | | | dehydrogenase/reductase family | |
| | | | [Mycobacterium tuberculosis CDC1551] | |
| | | | >gb\|EFO75870.1\|short-chain type | |
| | | | dehydrogenase/reductase [Mycobacterium | |
| | | | tuberculosis SUMu001] >gb\|EFP48221.1\| | |
| | | | short-chain type dehydrogenase/reductase | |
| | | | [Mycobacterium tuberculosis SUMu010] | |
| | | | >gb\|EFP52129.1\|short-chain type | |
| | | | dehydrogenase/reductase [Mycobacterium | |
| | | | tuberculosis SUMu011] | |
| 879 | 880 | ZP_01627272.1 | short-chain dehydrogenase/reductase SDR | LigD |
| | | | [marine gamma proteobacterium | |
| | | | HTCC2080] >gb\|EAW39988.1\|short-chain | |
| | | | dehydrogenase/reductase SDR [marine | |
| | | | gamma proteobacterium HTCC2080] | |
| 881 | 882 | YP_002774647.1 | short chain dehydrogenase [Brevibacillus | LigD |
| | | | brevis NBRC 100599] >dbj\|BAH46143.1\| | |
| | | | probable short chain dehydrogenase | |
| | | | [Brevibacillus brevis NBRC 100599] | |
| 883 | 884 | YP_004533909.1 | short-chain dehydrogenase/reductase SDR | LigD |
| | | | [Novosphingobium sp. PP1Y] | |
| | | | >emb\|CCA92091.1\|short-chain | |
| | | | dehydrogenase/reductase SDR | |
| | | | [Novosphingobium sp. PP1Y] | |
| 885 | 886 | ZP_04751842.1 | short chain dehydrogenase [Mycobacterium | LigD |
| | | | kansasii ATCC 12478] | |
| 887 | 888 | ZP_08271356.1 | short-chain dehydrogenase/reductase SDR | LigD |
| | | | [gamma proteobacterium IMCC3088] | |
| | | | >gb\|EGG29327.1\|short-chain | |
| | | | dehydrogenase/reductase SDR [gamma | |
| | | | proteobacterium IMCC3088] | |
| 889 | 890 | YP_004666338.1 | short chain dehydrogenase [Myxococcus | LigD |
| | | | fulvus HW-1] >gb\|AEI65260.1\|short chain | |
| | | | dehydrogenase [Myxococcus fulvus HW-1] | |
| 891 | 892 | YP_001704647.1 | putative short chain | LigD |
| | | | dehydrogenase/reductase [Mycobacterium | |
| | | | abscessus ATCC 19977] | |
| | | | >emb\|CAM63993.1\|Putative short chain | |
| | | | dehydrogenase/reductase [Mycobacterium | |
| | | | abscessus] | |
| 893 | 894 | ZP_07283949.1 | cis-2,3-dihydrobiphenyl-2,3-diol | LigD |
| | | | dehydrogenase [Streptomyces sp. AA4] | |
| | | | >gb\|EFL12318.1\|cis-2,3-dihydrobiphenyl- | |
| | | | 2,3-diol dehydrogenase [Streptomyces sp. | |
| | | | AA4] | |
| 895 | 896 | YP_002005492.1 | hypothetical protein RALTA_A1476 | LigD |
| | | | [Cupriavidus taiwanensis LMG 19424] | |
| | | | >emb\|CAQ69425.1\|putative | |
| | | | OXIDOREDUCTASE DEHYDROGENASE | |
| | | | [Cupriavidus taiwanensis LMG 19424] | |
| 897 | 898 | YP_003543705.1 | SDR-family protein [Sphingobium japonicum | LigD |
| | | | UT26S] >dbj\|BAI95093.1\|SDR-family | |
| | | | protein [Sphingobium japonicum UT26S] | |
| 899 | 900 | YP_759628.1 | short chain dehydrogenase/reductase family | LigD |
| | | | oxidoreductase [Hyphomonas neptunium | |
| | | | ATCC 15444] >gb\|ABI75402.1\| | |
| | | | oxidoreductase, short chain | |
| | | | dehydrogenase/reductase family | |
| | | | [Hyphomonas neptunium ATCC 15444] | |
| 901 | 902 | ZP_03543905.1 | short-chain dehydrogenase/reductase SDR | LigD |
| | | | [Comamonas testosteroni KF-1] | |
| | | | >gb\|EED68191.1\|short-chain | |
| | | | dehydrogenase/reductase SDR | |
| | | | [Comamonas testosteroni KF-1] | |
| 903 | 904 | YP_003487191.1 | hypothetical protein SCAB_14801 | LigD |
| | | | [Streptomyces scabiei 87.22] | |
| | | | >emb\|CBG68626.1\|putative PROBABLE | |
| | | | SHORT-CHAIN TYPE | |
| | | | DEHYDROGENASE/REDUCTASE | |
| | | | [Streptomyces scabiei 87.22] | |
| 905 | 906 | AEG69105.1 | 3-oxoacyl-[acyl-carrier-protein] reductase | LigD |
| | | | [Ralstonia solanacearum Po82] | |
| 907 | 908 | YP_003841993.1 | short-chain dehydrogenase/reductase SDR | LigD |
| | | | [Clostridium cellulovorans 743B] | |
| | | | >ref\|ZP_07630916.1\|short-chain | |
| | | | dehydrogenase/reductase SDR [Clostridium | |
| | | | cellulovorans 743B] >gb\|ADL50229.1\|short- | |
| | | | chain dehydrogenase/reductase SDR | |
| | | | [Clostridium cellulovorans 743B] | |
| 909 | 910 | YP_001899010.1 | hypothetical protein Rpic_1437 [Ralstonia | LigD |
| | | | pickettii 12J] >gb\|ACD26578.1\|short-chain | |
| | | | dehydrogenase/reductase SDR [Ralstonia | |
| | | | pickettii 12J] | |
| 911 | 912 | ZP_07965490.1 | short chain dehydrogenase [Segniliparus | LigD |
| | | | rugosus ATCC BAA-974] >gb\|EFV13275.1\| | |
| | | | short chain dehydrogenase [Segniliparus | |
| | | | rugosus ATCC BAA-974] | |
| 913 | 914 | NP_250228.1 | short-chain dehydrogenase [Pseudomonas | LigD |
| | | | aeruginosa PAO1] >ref\|ZP_01364886.1\| | |
| | | | hypothetical protein PaerPA_01001998 | |
| | | | [Pseudomonas aeruginosa PACS2] | |
| | | | >ref\|YP_002441374.1\|putative short-chain | |
| | | | dehydrogenase [Pseudomonas aeruginosa | |
| | | | LESB58] >ref\|ZP_04933207.1\|hypothetical | |
| | | | protein PA2G_00514 [Pseudomonas | |
| | | | aeruginosa 2192] | |
| | | | >gb\|AAG04926.1\|AE004582_4 probable | |
| | | | short-chain dehydrogenase [Pseudomonas | |
| | | | aeruginosa PAO1] >gb\|EAZ57326.1\| | |
| | | | hypothetical protein PA2G_00514 | |
| | | | [Pseudomonas aeruginosa 2192] | |
| | | | >emb\|CAW28518.1\|probable short-chain | |
| | | | dehydrogenase [Pseudomonas aeruginosa | |
| | | | LESB58] >gb\|EGM16253.1\|putative short- | |
| | | | chain dehydrogenase [Pseudomonas | |
| | | | aeruginosa 138244] | |
| 915 | 916 | YP_001020978.1 | hypothetical protein Mpe_A1784 | LigD |
| | | | [Methylibium petroleiphilum PM1] | |
| | | | >gb\|ABM94743.1\|putative oxidoreductase | |
| | | | dehydrogenase signal peptide protein | |
| | | | [Methylibium petroleiphilum PM1] | |
| 917 | 918 | YP_003745682.1 | oxidoreductase dehydrogenase [Ralstonia | LigD |
| | | | solanacearum CFBP2957] | |
| | | | >emb\|CBJ43067.1\|putative oxidoreductase | |
| | | | dehydrogenase [Ralstonia solanacearum | |
| | | | CFBP2957] | |
| 919 | 920 | ADD82954.1 | BatM [Pseudomonas fluorescens] | LigD |
| 921 | 922 | ZP_06846575.1 | short-chain dehydrogenase/reductase family | LigD |
| | | | oxidoreductase [Mycobacterium | |
| | | | parascrofulaceum ATCC BAA-614] | |
| | | | >gb\|EFG80090.1\|short-chain | |
| | | | dehydrogenase/reductase family | |
| | | | oxidoreductase [Mycobacterium | |
| | | | parascrofulaceum ATCC BAA-614] | |
| 923 | 924 | ZP_05041687.1 | oxidoreductase, short chain | LigD |
| | | | dehydrogenase/reductase family | |
| | | | [Alcanivorax sp. DG881] >gb\|EDX89108.1\| | |
| | | | oxidoreductase, short chain | |
| | | | dehydrogenase/reductase family | |
| | | | [Alcanivorax sp. DG881] | |
| 925 | 926 | YP_726036.1 | hypothetical protein H16_A1536 [Ralstonia | LigD |
| | | | eutropha H16] >emb\|CAJ92668.1\| | |
| | | | conserved hypothetical protein [Ralstonia | |
| | | | eutropha H16] | |
| 927 | 928 | ZP_08275744.1 | Hypothetical Protein IMCC9480_775 | LigD |
| | | | [Oxalobacteraceae bacterium IMCC9480] | |
| | | | >gb\|EGF30787.1\|Hypothetical Protein | |
| | | | IMCC9480_775 [Oxalobacteraceae | |
| | | | bacterium IMCC9480] | |
| 929 | 930 | YP_791716.1 | putative short-chain dehydrogenase | LigD |
| | | | [Pseudomonas aeruginosa UCBPP-PA14] | |
| | | | >ref\|ZP_06879570.1\|putative short-chain | |
| | | | dehydrogenase [Pseudomonas aeruginosa | |
| | | | PAb1] >ref\|ZP_07792770.1\|putative short- | |
| | | | chain dehydrogenase [Pseudomonas | |
| | | | aeruginosa 39016] >gb\|ABJ10717.1\| | |
| | | | putative short-chain dehydrogenase | |
| | | | [Pseudomonas aeruginosa UCBPP-PA14] | |
| | | | >gb\|EFQ37866.1\|putative short-chain | |
| | | | dehydrogenase [Pseudomonas aeruginosa | |
| | | | 39016] >gb\|EGM15719.1\|putative short- | |
| | | | chain dehydrogenase [Pseudomonas | |
| | | | aeruginosa 152504] | |
| 931 | 932 | CAQ35702.1 | oxidoreductase dehydrogenase protein | LigD |
| | | | [Ralstonia solanacearum MolK2] | |
| 933 | 934 | ZP_07966320.1 | short chain dehydrogenase [Segniliparus | LigD |
| | | | rugosus ATCC BAA-974] >gb\|EFV12481.1\| | |
| | | | short chain dehydrogenase [Segniliparus | |
| | | | rugosus ATCC BAA-974] | |
| 935 | 936 | YP_002981437.1 | hypothetical protein Rpic12D_1478 | LigD |
| | | | [Ralstonia pickettii 12D] >gb\|ACS62765.1\| | |
| | | | short-chain dehydrogenase/reductase SDR | |
| | | | [Ralstonia pickettii 12D] | |
| 937 | 938 | YP_004685391.1 | C alpha-dehydrogenase LigD [Cupriavidus | LigD |
| | | | necator N-1] >gb\|AEI76910.1\|C alpha- | |
| | | | dehydrogenase LigD [Cupriavidus necator | |
| | | | N-1] | |
| 939 | 940 | ZP_00945631.1 | Hypothetical Protein RRSL_01608 | LigD |
| | | | [Ralstonia solanacearum UW551] | |
| | | | >ref\|YP_002259522.1\|oxidoreductase | |
| | | | dehydrogenase protein [Ralstonia | |
| | | | solanacearum IPO1609] >gb\|EAP71895.1\| | |
| | | | Hypothetical Protein RRSL_01608 | |
| | | | [Ralstonia solanacearum UW551] | |
| | | | >emb\|CAQ61454.1\|oxidoreductase | |
| | | | dehydrogenase protein [Ralstonia | |
| | | | solanacearum IPO1609] | |
| 941 | 942 | NP_519890.1 | hypothetical protein RSc1769 [Ralstonia | LigD |
| | | | solanacearum GMI1000] | |
| | | | >emb\|CAD15471.1\|probable | |
| | | | oxidoreductase dehydrogenase signal | |
| | | | peptide protein [Ralstonia solanacearum | |
| | | | GMI1000] | |
| 943 | 944 | ZP_07676733.1 | oxidoreductase dehydrogenase signal | LigD |
| | | | peptide protein [Ralstonia sp. 5_7_47FAA] | |
| | | | >gb\|EFP64736.1\|oxidoreductase | |
| | | | dehydrogenase signal peptide protein | |
| | | | [Ralstonia sp. 5_7_47FAA] | |
| 945 | 946 | YP_003752456.1 | oxidoreductase dehydrogenase [Ralstonia | LigD |
| | | | solanacearum PSI07] >emb\|CBJ51176.1\| | |
| | | | putative oxidoreductase dehydrogenase | |
| | | | [Ralstonia solanacearum PSI07] | |
| 947 | 948 | YP_004533099.1 | hypothetical protein PP1Y_AT3242 | LigD |
| | | | [Novosphingobium sp. PP1Y] | |
| | | | >emb\|CCA91281.1\|conserved hypothetical | |
| | | | protein [Novosphingobium sp. PP1Y] | |
| 949 | 950 | YP_001564386.1 | hypothetical protein Daci_3363 [Delftia | LigD |
| | | | acidovorans SPH-1] >gb\|ABX36001.1\|short- | |
| | | | chain dehydrogenase/reductase SDR | |
| | | | [Delftia acidovorans SPH-1] | |
| 951 | 952 | YP_004488753.1 | short-chain dehydrogenase/reductase SDR | LigD |
| | | | [Delftia sp. Cs1-4] >gb\|AEF90398.1\|short- | |
| | | | chain dehydrogenase/reductase SDR | |
| | | | [Delftia sp. Cs1-4] | |
| 953 | 954 | YP_001188109.1 | short-chain dehydrogenase/reductase SDR | LigD |
| | | | [Pseudomonas mendocina ymp] | |
| | | | >gb\|ABP85377.1\|short-chain | |
| | | | dehydrogenase/reductase SDR | |
| | | | [Pseudomonas mendocina ymp] | |
| 955 | 956 | ADP99633.1 | short-chain dehydrogenase/reductase SDR | LigD |
| | | | [Marinobacter adhaerens HP15] | |
| 957 | 958 | YP_693638.1 | short-chain dehydrogenase/reductase family | LigD |
| | | | protein [Alcanivorax borkumensis SK2] | |
| | | | >emb\|CAL17366.1\|short-chain | |
| | | | dehydrogenase/reductase family | |
| | | | [Alcanivorax borkumensis SK2] | |
| 959 | 960 | YP_585740.1 | short-chain dehydrogenase/reductase SDR | LigD |
| | | | [Cupriavidus metallidurans CH34] | |
| | | | >gb\|ABF10471.1\|short-chain | |
| | | | dehydrogenase/reductase SDR [Cupriavidus | |
| | | | metallidurans CH34] | |
| 961 | 962 | YP_003277769.1 | short-chain dehydrogenase/reductase SDR | LigD |
| | | | [Comamonas testosteroni CNB-2] | |
| | | | >gb\|ACY32473.1\|short-chain | |
| | | | dehydrogenase/reductase SDR | |
| | | | [Comamonas testosteroni CNB-2] | |
| 963 | 964 | ZP_08406457.1 | hypothetical protein HGR_11311 | LigD |
| | | | [Hylemonella gracilis ATCC 19624] | |
| | | | >gb\|EGI76405.1\|hypothetical protein | |
| | | | HGR_11311 [Hylemonella gracilis ATCC | |
| | | | 19624] | |
| 965 | 966 | YP_003842521.1 | short-chain dehydrogenase/reductase SDR | LigD |
| | | | [Clostridium cellulovorans 743B] | |
| | | | >ref\|ZP_07632312.1\|short-chain | |
| | | | dehydrogenase/reductase SDR [Clostridium | |
| | | | cellulovorans 743B] >gb\|ADL50757.1\|short- | |
| | | | chain dehydrogenase/reductase SDR | |
| | | | [Clostridium cellulovorans 743B] | |
| 967 | 968 | ZP_07043693.1 | short-chain dehydrogenase/reductase SDR | LigD |
| | | | [Comamonas testosteroni S44] | |
| | | | >gb\|EFI62855.1\|short-chain | |
| | | | dehydrogenase/reductase SDR | |
| | | | [Comamonas testosteroni S44] | |
| 969 | 970 | YP_295629.1 | hypothetical protein Reut_A1415 [Ralstonia | LigD |
| | | | eutropha JMP134] >gb\|AAZ60785.1\|Short- | |
| | | | chain dehydrogenase/reductase SDR | |
| | | | [Ralstonia eutropha JMP134] | |
| 971 | 972 | CBJ37979.1 | putative oxidoreductase dehydrogenase | LigD |
| | | | [Ralstonia solanacearum CMR15] | |
| 973 | 974 | YP_004155471.1 | short-chain dehydrogenase/reductase sdr | LigD |
| | | | [Variovorax paradoxus EPS] | |
| | | | >gb\|ADU37360.1\|short-chain | |
| | | | dehydrogenase/reductase SDR [Variovorax | |
| | | | paradoxus EPS] | |
| 975 | 976 | YP_001353681.1 | hypothetical protein mma_1991 | LigD |
| | | | [Janthinobacterium sp. Marseille] | |
| | | | >gb\|ABR91341.1\|short-chain | |
| | | | dehydrogenase/reductase SDR | |
| | | | [Janthinobacterium sp. Marseille] | |

[symbol]

indicates data missing or illegible when filed

Those skilled in the art will recognize, or be able to ascertain using no more than routine experimentation, that there are many equivalents to the specific embodiments described herein that have been described and enabled to the extent that one of skill in the art can practice the invention well-beyond the scope of the specific embodiments taught herein. Such equivalents are intended to be encompassed by the following claims. In addition, there are numerous lists and Markush groups taught and claimed herein. One of skill will appreciate that each such list and group contains various species and can be modified by the removal, or addition, of one or more of species, since every list and group taught and claimed herein may not be applicable to every embodiment feasible in the practice of the invention. As such, components in such lists can be removed and are expected to be removed to reflect some embodiments taught herein. All publications, patents, patent applications, other references, accession numbers, ATCC numbers, etc., mentioned in this application are herein incorporated by reference into the specification to the same extent as if each was specifically indicated to be herein incorporated by reference in its entirety.

We claim:

1. A recombinant polynucleotide comprising a nucleotide sequence that encodes a polypeptide comprising an amino acid sequence having at least 95% identity to SEQ ID NO:101, the amino acid sequence conserving residues 19-22, 24, 25, 27-30, 33-36, 39-45, 47, 48, 50-54; 100, 101, 104, 111, 112, 115, 116, 166, 107, 184, 187, 188, 191, 192, and 195 of SEQ ID:101.

2. A recombinant polynucleotide comprising a nucleotide sequence that encodes a polypeptide comprising SEQ ID NO:101; or conservative substitutions thereof outside of conserved residues 19-22, 24, 25, 27-30, 33-36, 39-45, 47, 48, 50-54; 100, 101, 104, 111, 112, 115, 116, 166, 107, 184, 187, 188, 191, 192, and 195 of SEQ ID:101.

3. A vector comprising the polynucleotide of claim 1.

4. A vector comprising the polynucleotide of claim 2.

5. A plasmid comprising the polynucleotide of claim 1.

6. A plasmid comprising the polynucleotide of claim 2.

7. A host cell transformed by the vector of claim 3.

8. A host cell transformed by the vector of claim 4.

9. A method of cleaving a beta-aryl ether bond, comprising
culturing the host cell of claim 7 under conditions suitable to produce the polypeptide;
recovering the polypeptide from the host cell culture; and,
contacting the polypeptide with a lignin-derived compound having (i) a beta-aryl ether bond and (ii) a molecular weight ranging from about 180 Daltons to about 3000 Daltons;
wherein, the contacting occurs in a solvent environment in which the lignin-derived compound is soluble.

10. The method of claim 9, wherein the host cell is E. Coli.

11. The method of claim 9, wherein the host cell is Azotobacter vinelandii.

12. The method of claim 9, wherein the lignin-derived compound has a molecular weight of about 180 Daltons to about 1000 Daltons.

13. The method of claim 9, wherein an amino acid substitution outside of the conserved residues is a conservative substitution.

14. The method of claim 9, wherein the solvent environment comprises water.

15. The method of claim 9, wherein the solvent environment comprises a polar organic solvent.

16. A method of cleaving a beta-aryl ether bond, comprising
culturing the host cell of claim 8 under conditions suitable to produce the polypeptide;
recovering the polypeptide from the host cell culture; and,
contacting the polypeptide with a lignin-derived compound having (i) a beta-aryl ether bond and (ii) a molecular weight ranging from about 180 Daltons to about 3000 Daltons;
wherein, the contacting occurs in a solvent environment in which the lignin-derived compound is soluble.

17. The method of claim 16, wherein the host cell is E. Coli.

18. The method of claim 16, wherein the host cell is Azotobacter vinelandii.

19. The method of claim 16, wherein the lignin-derived compound has a molecular weight of about 180 Daltons to about 1000 Daltons.

20. The method of claim 16, wherein the solvent environment comprises water.

21. The method of claim 16, wherein the solvent environment comprises a polar organic solvent.
